SEQUENCE LISTING (51 sequences)

SEQ ID NO: 1 - protein, 119 aa, Artificial Sequence
  Synthetic Construct
EVQLQQSGAELVKPGASVKLSCTASGFNIKDTYMYWVKQRPEQGLECIGRIDPANDNTKYDPKFQGKATITADTSSNTAYVQLASLTSEDTAVYYCARAKNLLNYFDYWGQGTTLTVSS

SEQ ID NO: 2 - DNA, 357 bp, Artificial Sequence
  Synthetic Construct
gaggttcagctgcagcagtctggggcagagcttgtgaagccaggggcctcagtcaagttgtcctgcacagcttctggcttcaacattaaagacacctatatgtactgggtgaagcagaggcctgaacagggcctggagtgcattggaaggattgatcctgcgaatgataatactaaatatgacccgaagttccagggcaaggccactataacagcagacacatcctccaacacagcctacgtgcagctcgccagtctgacatctgaggacactgccgtctattactgtgctagagcgaagaatttgttaaattactttgactactggggccaaggcaccactctcacagtctcctca

SEQ ID NO: 3 - protein, 108 aa, Artificial Sequence
  Synthetic Construct
DIQMTQSPSSLSASLGERVTLSCRASQEISGYLSWLQQKPDGTIKRLIYATSTLDSGVPKRFSGSRSGSDYSLTISSLESEDFADYYCLQYAIYPLTFGAGTKLELKR

SEQ ID NO: 4 - DNA, 324 bp, Artificial Sequence
  Synthetic Construct
gacatccagatgacccagtctccatcctccttatctgcctctctgggagaaagagtcactctcagttgtcgggcaagtcaggaaattagtggttacttaagctggcttcagcagaaaccagatggaactattaaacgcctgatctacgccacatcgactttagattctggtgtcccaaaaaggttcagtggcagtaggtctgggtcagattattctctcaccatcagcagccttgagtctgaagattttgcagactattactgtctacaatatgctatttatccgctcacgttcggtgctgggaccaagctggagctgaaacgg

SEQ ID NO: 5 - protein, 448 aa, Artificial Sequence
  Synthetic Construct
EVQLQQSGAELVKPGASVKLSCTASGFNIKDTYMYWVKQRPEQGLECIGRIDPANDNTKYDPKFQGKATITADTSSNTAYVQLASLTSEDTAVYYCARAKNLLNYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 6 - DNA, 1350 bp, Artificial Sequence
  Synthetic Construct
gaggttcagctgcagcagtctggggcagagcttgtgaagccaggggcctcagtcaagttgtcctgcacagcttctggcttcaacattaaagacacctatatgtactgggtgaagcagaggcctgaacagggcctggagtgcattggaaggattgatcctgcgaatgataatactaaatatgacccgaagttccagggcaaggccactataacagcagacacatcctccaacacagcctacgtgcagctcgccagtctgacatctgaggacactgccgtctattactgtgctagagcgaagaatttgttaaattactttgactactggggccaaggcaccactctcacagtctcctcagctagcaccaagggccccagcgtgttccctctggcccccagcagcaagagcaccagcggcggaaccgccgccctgggctgcctggtgaaggactacttccccgagcccgtgaccgtgtcctggaacagcggcgctctgaccagcggagtgcacaccttccctgccgtgctgcagagcagcggcctgtactccctgagcagcgtggtgaccgtgcccagcagcagcctgggcacccagacctacatctgcaacgtgaaccacaagccctccaacaccaaggtggacaagaaggtggagcctaagagctgcgacaagacccacacctgccctccctgccccgcccccgagctgctgggcggacccagcgtgttcctgttccctcccaagcccaaggacaccctgatgatcagccgcacccccgaggtgacctgcgtggtggtggacgtgagccacgaggaccccgaggtgaagttcaactggtacgtggacggcgtggaggtgcacaacgccaagaccaagcctcgggaggagcagtacaactccacctaccgcgtggtgagcgtgctgaccgtgctgcaccaggactggctgaacggcaaggagtacaagtgcaaggtgagcaacaaggccctgcccgctcccatcgagaagaccatcagcaaggccaagggccagccccgggagcctcaggtgtacaccctgccccccagccgcgacgagctgaccaagaaccaggtgagcctgacctgcctggtgaagggcttctacccctccgacatcgccgtggagtgggagagcaacggccagcctgagaacaactacaagaccacccctcccgtgctggacagcgacggcagcttcttcctgtacagcaagctgaccgtggacaagtcccggtggcagcagggcaacgtgttcagctgcagcgtgatgcacgaggccctgcacaaccactacacccagaagagcctgagcctgagccccggatagtaa

SEQ ID NO: 7 - protein, 214 aa, Artificial Sequence
  Synthetic Construct
DIQMTQSPSSLSASLGERVTLSCRASQEISGYLSWLQQKPDGTIKRLIYATSTLDSGVPKRFSGSRSGSDYSLTISSLESEDFADYYCLQYAIYPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - DNA, 645 bp, Artificial Sequence
  Synthetic Construct
gacatccagatgacccagtctccatcctccttatctgcctctctgggagaaagagtcactctcagttgtcgggcaagtcaggaaattagtggttacttaagctggcttcagcagaaaccagatggaactattaaacgcctgatctacgccacatcgactttagattctggtgtcccaaaaaggttcagtggcagtaggtctgggtcagattattctctcaccatcagcagccttgagtctgaagattttgcagactattactgtctacaatatgctatttatccgctcacgttcggtgctgggaccaagctggagctgaaacggaccgtggccgcccccagcgtgttcatcttccctcccagcgacgagcagctgaagtctggcaccgccagcgtggtgtgcctgctgaacaacttctacccccgcgaggccaaggtgcagtggaaggtggacaacgccctgcagagcggcaacagccaggagagcgtgaccgagcaggactccaaggacagcacctacagcctgagcagcaccctgaccctgagcaaggccgactacgagaagcacaaggtgtacgcctgcgaggtgacccaccagggactgtctagccccgtgaccaagagcttcaaccggggcgagtgctaa

SEQ ID NO: 9 - protein, 119 aa, Artificial Sequence
  Synthetic Construct
QVQLVQSGAEVKKPGASVKVSCKASGFNIKDTYMYWVRQAPGQGLEWMGRIDPANDNTKYAQKFQGRVTITADTSTSTAYMELSSLRSEDTAVYYCARAKNLLNYFDYWGQGTLVTVSS

SEQ ID NO: 10 - DNA, 357 bp, Artificial Sequence
  Synthetic Construct
caggttcagctggtgcagtctggggcagaggttaagaagccaggggcctcagtcaaggtgtcctgcaaagcttctggcttcaacattaaagacacctatatgtactgggtgaggcaggcacctggacagggcctggagtggatgggaaggattgatcctgcgaatgataatactaaatatgcccagaagttccagggcagggtcactataacagcagacacatccaccagcacagcctacatggagctctccagtctgagatctgaggacactgccgtctattactgtgctagagcgaagaatttgttaaattactttgactactggggccaaggcacccttgtcacagtctcctca

SEQ ID NO: 11 - protein, 119 aa, Artificial Sequence
  Synthetic Construct
QVQLVQSGAEVKKPGASVKVSCKASGFNIKDTYMYWVRQAPGQGLEWIGRIDPANDNTKYAPKFQGRVTITADTSTNTAYMELSSLRSEDTAVYYCARAKNLLNYFDYWGQGTLVTVSS

SEQ ID NO: 12 - DNA, 357 bp, Artificial Sequence
  Synthetic Construct
caggttcagctggtgcagtctggggcagaggttaagaagccaggggcctcagtcaaggtgtcctgcaaagcttctggcttcaacattaaagacacctatatgtactgggtgaggcaggcacctggacagggcctggagtggattggaaggattgatcctgcgaatgataatactaaatatgccccgaagttccagggcagggtcactataacagcagacacatccaccaacacagcctacatggagctctccagtctgagatctgaggacactgccgtctattactgtgctagagcgaagaatttgttaaattactttgactactggggccaaggcacccttgtcacagtctcctca

SEQ ID NO: 13 - protein, 119 aa, Artificial Sequence
  Synthetic Construct
EVQLVQSGAEVKKPGASVKVSCKASGFNIKDTYMYWVRQAPGQGLEWMGRIDPANDNTKYAQKFQGRVTITADTSTNTAYMELSSLRSEDTAVYYCARAKNLLNYFDYWGQGTLVTVSS

SEQ ID NO: 14 - DNA, 357 bp, Artificial Sequence
  Synthetic Construct
gaggttcagctggtgcagtctggggcagaggttaagaagccaggggcctcagtcaaggtgtcctgcaaagcttctggcttcaacattaaagacacctatatgtactgggtgaggcaggcacctggacagggcctggagtggatgggaaggattgatcctgcgaatgataatactaaatatgcccagaagttccagggcagggtcactataacagcagacacatccaccaacacagcctacatggagctctccagtctgagatctgaggacactgccgtctattactgtgctagagcgaagaatttgttaaattactttgactactggggccaaggcacccttgtcacagtctcctca

SEQ ID NO: 15 - protein, 108 aa, Artificial Sequence
  Synthetic Construct
DIQMTQSPSSLSASVGDRVTITCRASQEISGYLSWYQQKPGKAPKRLIYATSTLDSGVPSRFSGSGSGTDFTLTISSLQPEDFATYYCLQYAIYPLTFGQGTKLEIKR

SEQ ID NO: 16 - DNA, 309 bp, Artificial Sequence
  Synthetic Construct
gacatccagatgacccagtctccatcctccttatctgcctctgtgggagatagagtcactatcacttgtcgggcaagtcaggaaattagtggttacttaagctggtatcagcagaaaaaacgcctgatctacgccacatcgactttagattctggtgtcccaagtaggttcagtggcagtgggtctgggacagattttactctcaccatcagcagccttcagcctgaagattttgcaacctattactgtctacaatatgctatttatccgctcacgttcggtcaggggaccaagctggagatcaaacgg

SEQ ID NO: 17 - protein, 108 aa, Artificial Sequence
  Synthetic Construct
DIQMTQSPSSLSASVGDRVTITCRASQEISGYLSWLQQKPGKAPKRLIYATSTLQSGVPSRFSGSRSGTDYTLTISSLQPEDFATYYCLQYAIYPLTFGQGTKLEIKR

SEQ ID NO: 18 - DNA, 324 bp, Artificial Sequence
  Synthetic Construct
gacatccagatgacccagtctccatcctccttatctgcctctgtgggagatagagtcactatcacttgtcgggcaagtcaggaaattagtggttacttaagctggcttcagcagaaaccaggtaaggctcctaaacgcctgatctacgccacatcgactttacagtctggtgtcccaagtaggttcagtggcagtaggtctgggacagattatactctcaccatcagcagccttcagcctgaagattttgcaacctattactgtctacaatatgctatttatccgctcacgttcggtcaggggaccaagctggagatcaaacgg

SEQ ID NO: 19 - protein, 108 aa, Artificial Sequence
  Synthetic Construct
DIQMTQSPSSLSASVGDRVTITCRASQEISGYLSWYQQKPGKAPKRLIYATSTLDSGVPSRFSGSRSGSDYTLTISSLQPEDFATYYCLQYAIYPLTFGQGTKLEIKR

SEQ ID NO: 20 - DNA, 324 bp, Artificial Sequence
  Synthetic Construct
gacatccagatgacccagtctccatcctccttatctgcctctgtgggagatagagtcactatcacttgtcgggcaagtcaggaaattagtggttacttaagctggtatcagcagaaaccaggtaaggctcctaaacgcctgatctacgccacatcgactttagattctggtgtcccaagtaggttcagtggcagtaggtctgggtcagattatactctcaccatcagcagccttcagcctgaagattttgcaacctattactgtctacaatatgctatttatccgctcacgttcggtcaggggaccaagctggagatcaaacgg

SEQ ID NO: 21 - protein, 471 aa, Artificial Sequence
  Synthetic Construct
QVQLVQSGAEVKKPGASVKVSCKASGFNIKDTYMYWVRQAPGQGLEWMGRIDPANDNTKYAQKFQGRVTITADTSTSTAYMELSSLRSEDTAVYYCARAKNLLNYFDYWGQGTLVTVSSGSTSGSGKPGSGEGSTKGDIQMTQSPSSLSASVGDRVTITCRASQEISGYLSWLQQKPGKAPKRLIYATSTLQSGVPSRFSGSRSGTDYTLTISSLQPEDFATYYCLQYAIYPLTFGQGTKLEIKRDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 22 - DNA, 1419 bp, Artificial Sequence
  Synthetic Construct
caggttcagctggtgcagtctggggcagaggttaagaagccaggggcctcagtcaaggtgtcctgcaaggcttctggcttcaacattaaagacacctatatgtactgggtgaggcaggcacctggacagggcctggagtggatgggaaggattgatcctgcgaatgataatactaaatatgcccagaagttccagggcagggtcactataacagcagacacatccaccagcacagcctacatggagctctccagtctgagatctgaggacactgccgtctattactgtgctagagcgaagaatttgttaaattactttgactactggggccaaggcacccttgtcacagtctcctcaggcagcacctccggcagcggcaagcctggcagcggcgagggcagcaccaagggcgacatccagatgacccagtctccatcctccttatctgcctctgtgggagatagagtcactatcacttgtcgggcaagtcaggaaattagtggttacttaagctggcttcagcagaaaccaggtaaggctcctaaacgcctgatctacgccacatcgactttacagtctggtgtcccaagtaggttcagtggcagtaggtctgggacagattatactctcaccatcagcagccttcagcctgaagattttgcaacctattactgtctacaatatgctatttatccgctcacgttcggtcaggggaccaagctggagatcaaacgggacaagacccacacctgccctccctgccccgcccccgagctgctgggcggacccagcgtgttcctgttccctcccaagcccaaggacaccctgatgatcagccgcacccccgaggtgacctgcgtggtggtggacgtgagccacgaggaccccgaggtgaagttcaactggtacgtggacggcgtggaggtgcacaacgccaagaccaagcctcgggaggagcagtacaactccacctaccgcgtggtgagcgtgctgaccgtgctgcaccaggactggctgaacggcaaggagtacaagtgcaaggtgagcaacaaggccctgcccgctcccatcgagaagaccatcagcaaggccaagggccagccccgggagcctcaggtgtacaccctgccccccagccgcgacgagctgaccaagaaccaggtgagcctgacctgcctggtgaagggcttctacccctccgacatcgccgtggagtgggagagcaacggccagcctgagaacaactacaagaccacccctcccgtgctggacagcgacggcagcttcttcctgtacagcaagctgaccgtggacaagtcccggtggcagcagggcaacgtgttcagctgcagcgtgatgcacgaggccctgcacaaccactacacccagaagagcctgagcctgagccccggataataa

SEQ ID NO: 23 - protein, 471 aa, Artificial Sequence
  Synthetic Construct
QVQLVQSGAEVKKPGASVKVSCKASGFNIKDTYMYWVRQAPGQGLEWMGRIDPANDNTKYAQKFQGRVTITADTSTSTAYMELSSLRSEDTAVYYCARAKNLLNYFDYWGQGTLVTVSSGSTSGSGKPGSGEGSTKGDIQMTQSPSSLSASVGDRVTITCRASQEISGYLSWLQQKPGKAPKRLIYATSTLQSGVPSRFSGSRSGTDYTLTISSLQPEDFATYYCLQYAIYPLTFGQGTKLEIKRDKTHTCPPCPAPELLGGPSVFLFPPKPKDTLMISRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNQYTQKSLSLSPG

SEQ ID NO: 24 - DNA, 1419 bp, Artificial Sequence
  Synthetic Construct
caggttcagctggtgcagtctggggcagaggttaagaagccaggggcctcagtcaaggtgtcctgcaaggcttctggcttcaacattaaagacacctatatgtactgggtgaggcaggcacctggacagggcctggagtggatgggaaggattgatcctgcgaatgataatactaaatatgcccagaagttccagggcagggtcactataacagcagacacatccaccagcacagcctacatggagctctccagtctgagatctgaggacactgccgtctattactgtgctagagcgaagaatttgttaaattactttgactactggggccaaggcacccttgtcacagtctcctcaggcagcacctccggcagcggcaagcctggcagcggcgagggcagcaccaagggcgacatccagatgacccagtctccatcctccttatctgcctctgtgggagatagagtcactatcacttgtcgggcaagtcaggaaattagtggttacttaagctggcttcagcagaaaccaggtaaggctcctaaacgcctgatctacgccacatcgactttacagtctggtgtcccaagtaggttcagtggcagtaggtctgggacagattatactctcaccatcagcagccttcagcctgaagattttgcaacctattactgtctacaatatgctatttatccgctcacgttcggtcaggggaccaagctggagatcaaacgggacaagacccacacctgccctccctgccccgcccccgagctgctgggcggacccagcgtgttcctgttccctcccaagcccaaggacaccctgatgatcagccgcacccccgaggtgacctgcgtggtggtggacgtgagccacgaggaccccgaggtgaagttcaactggtacgtggacggcgtggaggtgcacaacgccaagaccaagcctcgggaggagcagtacaactccacctaccgcgtggtgagcgtgctgaccgtgctggcccaggactggctgaacggcaaggagtacaagtgcaaggtgagcaacaaggccctgcccgctcccatcgagaagaccatcagcaaggccaagggccagccccgggagcctcaggtgtacaccctgccccccagccgcgacgagctgaccaagaaccaggtgagcctgacctgcctggtgaagggcttctacccctccgacatcgccgtggagtgggagagcaacggccagcctgagaacaactacaagaccacccctcccgtgctggacagcgacggcagcttcttcctgtacagcaagctgaccgtggacaagtcccggtggcagcagggcaacgtgttcagctgcagcgtgatgcacgaggccctgcacaaccagtacacccagaagagcctgagcctgagccccggataataa

SEQ ID NO: 25 - protein, 247 aa, Artificial Sequence
  Synthetic Construct
QVQLVQSGAEVKKPGASVKVSCKASGFNIKDTYMYWVRQAPGQGLEWMGRIDPANDNTKYAQKFQGRVTITADTSTSTAYMELSSLRSEDTAVYYCARAKNLLNYFDYWGQGTLVTVSSGGGGSGGGGSGGGGSGGGGSDIQMTQSPSSLSASVGDRVTITCRASQEISGYLSWLQQKPGKAPKRLIYATSTLQSGVPSRFSGSRSGTDYTLTISSLQPEDFATYYCLQYAIYPLTFGQGTKLEIKR

SEQ ID NO: 26 - DNA, 774 bp, Artificial Sequence
  Synthetic Construct
caggttcagctggtgcagtctggggcagaggttaagaagccaggggcctcagtcaaggtgtcctgcaaggcttctggcttcaacattaaagacacctatatgtactgggtgaggcaggcacctggacagggcctggagtggatgggaaggattgatcctgcgaatgataatactaaatatgcccagaagttccagggcagggtcactataacagcagacacatccaccagcacagcctacatggagctctccagtctgagatctgaggacactgccgtctattactgtgctagagcgaagaatttgttaaattactttgactactggggccaaggcacccttgtcacagtctcctcaggtggcggtggctctggaggtggtgggagcggtggcggcggatctgggggtggcggaagcgacatccagatgacccagtctccatcctccttatctgcctctgtgggagatagagtcactatcacttgtcgggcaagtcaggaaattagtggttacttaagctggcttcagcagaaaccaggtaaggctcctaaacgcctgatctacgccacatcgactttacagtctggtgtcccaagtaggttcagtggcagtaggtctgggacagattatactctcaccatcagcagccttcagcctgaagattttgcaacctattactgtctacaatatgctatttatccgctcacgttcggtcaggggaccaagctggagatcaaacggggaggtggtgggtctcaccatcaccatcaccat

SEQ ID NO: 27 - protein, 245 aa, Artificial Sequence
  Synthetic Construct
QVQLVQSGAEVKKPGASVKVSCKASGFNIKDTYMYWVRQAPGQGLEWMGRIDPANDNTKYAQKFQGRVTITADTSTSTAYMELSSLRSEDTAVYYCARAKNLLNYFDYWGQGTLVTVSSGSTSGSGKPGSGEGSTKGDIQMTQSPSSLSASVGDRVTITCRASQEISGYLSWLQQKPGKAPKRLIYATSTLQSGVPSRFSGSRSGTDYTLTISSLQPEDFATYYCLQYAIYPLTFGQGTKLEIKR

SEQ ID NO: 28 - DNA, 768 bp, Artificial Sequence
  Synthetic Construct
caggttcagctggtgcagtctggggcagaggttaagaagccaggggcctcagtcaaggtgtcctgcaaggcttctggcttcaacattaaagacacctatatgtactgggtgaggcaggcacctggacagggcctggagtggatgggaaggattgatcctgcgaatgataatactaaatatgcccagaagttccagggcagggtcactataacagcagacacatccaccagcacagcctacatggagctctccagtctgagatctgaggacactgccgtctattactgtgctagagcgaagaatttgttaaattactttgactactggggccaaggcacccttgtcacagtctcctcaggcagcacctccggcagcggcaagcctggcagcggcgagggcagcaccaagggcgacatccagatgacccagtctccatcctccttatctgcctctgtgggagatagagtcactatcacttgtcgggcaagtcaggaaattagtggttacttaagctggcttcagcagaaaccaggtaaggctcctaaacgcctgatctacgccacatcgactttacagtctggtgtcccaagtaggttcagtggcagtaggtctgggacagattatactctcaccatcagcagccttcagcctgaagattttgcaacctattactgtctacaatatgctatttatccgctcacgttcggtcaggggaccaagctggagatcaaacggggaggtggtgggtctcaccatcaccatcaccat

SEQ ID NO: 29 - protein, 247 aa, Artificial Sequence
  Synthetic Construct
QVQLVQSGAEVKKPGASVKVSCKASGFNIKDTYMYWVRQAPGQCLEWMGRIDPANDNTKYAQKFQGRVTITADTSTSTAYMELSSLRSEDTAVYYCARAKNLLNYFDYWGQGTLVTVSSGGGGSGGGGSGGGGSGGGGSDIQMTQSPSSLSASVGDRVTITCRASQEISGYLSWLQQKPGKAPKRLIYATSTLQSGVPSRFSGSRSGTDYTLTISSLQPEDFATYYCLQYAIYPLTFGCGTKLEIKR

SEQ ID NO: 30 - DNA, 774 bp, Artificial Sequence
  Synthetic Construct
caggttcagctggtgcagtctggggcagaggttaagaagccaggggcctcagtcaaggtgtcctgcaaggcttctggcttcaacattaaagacacctatatgtactgggtgaggcaggcacctggacagtgcctggagtggatgggaaggattgatcctgccaatgataatactaaatatgcccagaagttccagggcagggtcactataacagcagacacatccaccagcacagcctacatggagctctccagtctgagatctgaggacactgccgtctattactgtgctagagccaagaatttgttaaattactttgactactggggccaaggcacccttgtcacagtctcctcaggtggcggtggctctggaggtggtgggagcggtggcggcggatctgggggtggcggaagcgacatccagatgacccagtctccatcctccttatctgcctctgtgggagatagagtcactatcacttgtcgggcaagtcaggaaattagtggttacttaagctggcttcagcagaaaccaggtaaggctcctaaacgcctgatctacgccacatccactttacagtctggtgtcccaagtaggttcagtggcagtaggtctgggacagattatactctcaccatcagcagccttcagcctgaagattttgcaacctattactgtctacaatatgctatttatcctctcaccttcggttgtgggaccaagctggagatcaaacggggaggtggtgggtctcaccatcaccatcaccat

SEQ ID NO: 31 - protein, 247 aa, Artificial Sequence
  Synthetic Construct
QVQLVQSGAEVKKPGASVKVSCKASGFNIKDTYMYWVRQAPGQGLEWMGRIDPANDNTKYAQKFQGRVTITADTSTSTAYMELSSLRSEDTAVYYCARAKNLLNYFDYWGCGTLVTVSSGGGGSGGGGSGGGGSGGGGSDIQMTQSPSSLSASVGDRVTITCRASQEISGYLSWLQQKPGKCPKRLIYATSTLQSGVPSRFSGSRSGTDYTLTISSLQPEDFATYYCLQYAIYPLTFGQGTKLEIKR

SEQ ID NO: 32 - DNA, 774 bp, Artificial Sequence
  Synthetic Construct
caggttcagctggtgcagtctggggcagaggttaagaagccaggggcctcagtcaaggtgtcctgcaaggcttctggcttcaacattaaagacacctatatgtactgggtgaggcaggcacctggacagggcctggagtggatgggaaggattgatcctgcgaatgataatactaaatatgcccagaagttccagggcagggtcactataacagcagacacatccaccagcacagcctacatggagctctccagtctgagatctgaggacactgccgtctattactgtgctagagcgaagaatttgttaaattactttgactactggggctgcggcacccttgtcacagtctcctcaggtggcggtggctctggaggtggtgggagcggtggcggcggatctgggggtggcggaagcgacatccagatgacccagtctccatcctccttatctgcctctgtgggagatagagtcactatcacttgtcgggcaagtcaggaaattagtggttacttaagctggcttcagcagaaaccaggtaagtgtcctaaacgcctgatctacgccacatcgactttacagtctggtgtcccaagtaggttcagtggcagtaggtctgggacagattatactctcaccatcagcagccttcagcctgaagattttgcaacctattactgtctacaatatgctatttatccgctcacgttcggtcaggggaccaagctggagatcaaacggggaggtggtgggtctcaccatcaccatcaccat

SEQ ID NO: 33 - protein, 247 aa, Artificial Sequence
  Synthetic Construct
DIQMTQSPSSLSASVGDRVTITCRASQEISGYLSWLQQKPGKAPKRLIYATSTLQSGVPSRFSGSRSGTDYTLTISSLQPEDFATYYCLQYAIYPLTFGQGTKLEIKRGGGGSGGGGSGGGGSGGGGSQVQLVQSGAEVKKPGASVKVSCKASGFNIKDTYMYWVRQAPGQGLEWMGRIDPANDNTKYAQKFQGRVTITADTSTSTAYMELSSLRSEDTAVYYCARAKNLLNYFDYWGQGTLVTVSS

SEQ ID NO: 34 - DNA, 774 bp, Artificial Sequence
  Synthetic Construct
gacatccagatgacccagtctccatcctccttatctgcctctgtgggagatagagtcactatcacttgtcgggcaagtcaggaaattagtggttacttaagctggcttcagcagaaaccaggtaaggctcctaaacgcctgatctacgccacatcgactttacagtctggtgtcccaagtaggttcagtggcagtaggtctgggacagattatactctcaccatcagcagccttcagcctgaagattttgcaacctattactgtctacaatatgctatttatccgctcacgttcggtcaggggaccaagctggagatcaaacggggtggcggtggctctggaggtggtgggagcggtggcggcggatctgggggtggcggaagccaggttcagctggtgcagtctggggcagaggttaagaagccaggggcctcagtcaaggtgtcctgcaaggcttctggcttcaacattaaagacacctatatgtactgggtgaggcaggcacctggacagggcctggagtggatgggaaggattgatcctgcgaatgataatactaaatatgcccagaagttccagggcagggtcactataacagcagacacatccaccagcacagcctacatggagctctccagtctgagatctgaggacactgccgtctattactgtgctagagcgaagaatttgttaaattactttgactactggggccaaggcacccttgtcacagtctcctcaggaggcggagggtctcaccatcaccatcaccat

SEQ ID NO: 35 - protein, 245 aa, Artificial Sequence
  Synthetic Construct
DIQMTQSPSSLSASVGDRVTITCRASQEISGYLSWLQQKPGKAPKRLIYATSTLQSGVPSRFSGSRSGTDYTLTISSLQPEDFATYYCLQYAIYPLTFGQGTKLEIKRGSTSGSGKPGSGEGSTKGQVQLVQSGAEVKKPGASVKVSCKASGFNIKDTYMYWVRQAPGQGLEWMGRIDPANDNTKYAQKFQGRVTITADTSTSTAYMELSSLRSEDTAVYYCARAKNLLNYFDYWGQGTLVTVSS

SEQ ID NO: 36 - DNA, 768 bp, Artificial Sequence
  Synthetic Construct
gacatccagatgacccagtctccatcctccttatctgcctctgtgggagatagagtcactatcacttgtcgggcaagtcaggaaattagtggttacttaagctggcttcagcagaaaccaggtaaggctcctaaacgcctgatctacgccacatcgactttacagtctggtgtcccaagtaggttcagtggcagtaggtctgggacagattatactctcaccatcagcagccttcagcctgaagattttgcaacctattactgtctacaatatgctatttatccgctcacgttcggtcaggggaccaagctggagatcaaacggggcagcacctccggcagcggcaagcctggcagcggcgagggcagcaccaagggccaggttcagctggtgcagtctggggcagaggttaagaagccaggggcctcagtcaaggtgtcctgcaaggcttctggcttcaacattaaagacacctatatgtactgggtgaggcaggcacctggacagggcctggagtggatgggaaggattgatcctgcgaatgataatactaaatatgcccagaagttccagggcagggtcactataacagcagacacatccaccagcacagcctacatggagctctccagtctgagatctgaggacactgccgtctattactgtgctagagcgaagaatttgttaaattactttgactactggggccaaggcacccttgtcacagtctcctcaggaggcggagggtctcaccatcaccatcaccat

SEQ ID NO: 37 - protein, 247 aa, Artificial Sequence
  Synthetic Construct
DIQMTQSPSSLSASVGDRVTITCRASQEISGYLSWLQQKPGKCPKRLIYATSTLQSGVPSRFSGSRSGTDYTLTISSLQPEDFATYYCLQYAIYPLTFGQGTKLEIKRGGGGSGGGGSGGGGSGGGGSQVQLVQSGAEVKKPGASVKVSCKASGFNIKDTYMYWVRQAPGQGLEWMGRIDPANDNTKYAQKFQGRVTITADTSTSTAYMELSSLRSEDTAVYYCARAKNLLNYFDYWGCGTLVTVSS

SEQ ID NO: 38 - DNA, 774 bp, Artificial Sequence
  Synthetic Construct
gacatccagatgacccagtctccatcctccttatctgcctctgtgggagatagagtcactatcacttgtcgggcaagtcaggaaattagtggttacttaagctggcttcagcagaaaccaggtaagtgtcctaaacgcctgatctacgccacatcgactttacagtctggtgtcccaagtaggttcagtggcagtaggtctgggacagattatactctcaccatcagcagccttcagcctgaagattttgcaacctattactgtctacaatatgctatttatccgctcacgttcggtcaggggaccaagctggagatcaaacggggtggcggtggctctggaggtggtgggagcggtggcggcggatctgggggtggcggaagccaggttcagctggtgcagtctggggcagaggttaagaagccaggggcctcagtcaaggtgtcctgcaaggcttctggcttcaacattaaagacacctatatgtactgggtgaggcaggcacctggacagggcctggagtggatgggaaggattgatcctgcgaatgataatactaaatatgcccagaagttccagggcagggtcactataacagcagacacatccaccagcacagcctacatggagctctccagtctgagatctgaggacactgccgtctattactgtgctagagcgaagaatttgttaaattactttgactactggggctgtggcacccttgtcacagtctcctcaggaggcggagggtctcaccatcaccatcaccat

SEQ ID NO: 39 - protein, 247 aa, Artificial Sequence
  Synthetic Construct
DIQMTQSPSSLSASVGDRVTITCRASQEISGYLSWLQQKPGKAPKRLIYATSTLQSGVPSRFSGSRSGTDYTLTISSLQPEDFATYYCLQYAIYPLTFGCGTKLEIKRGGGGSGGGGSGGGGSGGGGSQVQLVQSGAEVKKPGASVKVSCKASGFNIKDTYMYWVRQAPGQCLEWMGRIDPANDNTKYAQKFQGRVTITADTSTSTAYMELSSLRSEDTAVYYCARAKNLLNYFDYWGQGTLVTVSS

SEQ ID NO: 40 - DNA, 774 bp, Artificial Sequence
  Synthetic Construct
gacatccagatgacccagtctccatcctccttatctgcctctgtgggagatagagtcactatcacttgtcgggcaagtcaggaaattagtggttacttaagctggcttcagcagaaaccaggtaaggctcctaaacgcctgatctacgccacatcgactttacagtctggtgtcccaagtaggttcagtggcagtaggtctgggacagattatactctcaccatcagcagccttcagcctgaagattttgcaacctattactgtctacaatatgctatttatccgctcacgttcggttgtgggaccaagctggagatcaaacggggtggcggtggctctggaggtggtgggagcggtggcggcggatctgggggtggcggaagccaggttcagctggtgcagtctggggcagaggttaagaagccaggggcctcagtcaaggtgtcctgcaaggcttctggcttcaacattaaagacacctatatgtactgggtgaggcaggcacctggacagtgcctggagtggatgggaaggattgatcctgcgaatgataatactaaatatgcccagaagttccagggcagggtcactataacagcagacacatccaccagcacagcctacatggagctctccagtctgagatctgaggacactgccgtctattactgtgctagagcgaagaatttgttaaattactttgactactggggccaaggcacccttgtcacagtctcctcaggaggcggagggtctcaccatcaccatcaccat

SEQ ID NO: 41 - protein, 8 aa, Artificial Sequence
  Synthetic Construct
GFNIKDTY

SEQ ID NO: 42 - protein, 8 aa, Artificial Sequence
  Synthetic Construct
IDPANDNT

SEQ ID NO: 43 - protein, 12 aa, Artificial Sequence
  Synthetic Construct
ARAKNLLNYFDY

SEQ ID NO: 44 - protein, 6 aa, Artificial Sequence
  Synthetic Construct
QEISGY

SEQ ID NO: 45 - protein, 3 aa, Artificial Sequence
  Synthetic Construct
ATS

SEQ ID NO: 46 - protein, 9 aa, Artificial Sequence
  Synthetic Construct
LQYAIYPLT

SEQ ID NO: 47 - protein, 20 aa, Artificial Sequence
  Synthetic Construct
GGGGSGGGGSGGGGSGGGGS

SEQ ID NO: 48 - protein, 18 aa, Artificial Sequence
  Synthetic Construct
GSTSGSGKPGSGEGSTKG

SEQ ID NO: 49 - protein, 290 aa, Artificial Sequence
  Synthetic Construct
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 50 - DNA, 30 bp, Artificial Sequence
  Synthetic Construct
gacaagacccacacctgccctccctgcccc

SEQ ID NO: 51 - protein, 11 aa, Artificial Sequence
  Synthetic Construct
GGGGSHHHHHH

(19) United States
(12) Patent Application Publication
(10) Pub. No.: US 2021/0309745 A1
(43) Pub. Date: Oct. 7, 2021

(54) COMPOSITIONS AND METHODS FOR IMAGING

(71) Applicant: Tayu Huaxia Biotech Medical Group Co., Ltd., Beijing (CN)

(72) Inventors: Lieping CHEN, Beijing (CN); Liqun LUO, Beijing (CN); Zhenguo WEN, Beijing (CN); Qianyong LIU, Beijing (CN)

(73) Assignee: Tayu Huaxia Biotech Medical Group Co., Ltd., Beijing (CN)

(21) Appl. No.: 17/263,037

(22) PCT Filed: Jul. 26, 2018
(86) PCT No.: PCT/CN2018/097175

§ 371 Date: Jan. 25, 2021

(51) Int. Cl. C07K 16/28; A61K 51/04

(52) U.S. Cl. CPC C07K 16/2827; A61K 2039/505; A61K 51/0482; A61K 51/0495

(57) ABSTRACT

The present application provides methods, imaging agents and kits for determination of the distribution and expression levels of an immune checkpoint ligand (such as PD-L1 or a PD-L1 like ligand) in an individual having a disease or condition. Anti-PD-L1 antibody agents are also provided.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 792572000240SEQLIST.TXT, date recorded: Jul. 23, 2018, size: 62 KB).

### FIELD OF THE INVENTION

The present invention relates to antibodies, imaging agents, and methods of imaging an immune checkpoint ligand.

### BACKGROUND OF THE INVENTION

The Programmed Death (PD) network involves at least five interacting molecules: PD-1 (Programmed Cell Death 1), two PD-1 ligands (PD-L1 and PD-L2), and two inhibitory receptors (PD-1 and CD80) of PD-L1. The crucial function of the PD pathway in modulating the activity of T cells in the peripheral tissues in an inflammatory response to infection and in limiting autoimmunity appears to be hijacked by tumor cells and by viruses during chronic viral infections. PD-L1 is overexpressed on many freshly isolated human tumors from multiple tissue origins (Dong et al. Nature Medicine 2002; 8:793-800; Romano et al. Journal for Immunotherapy of Cancer 2015; 3:15; Hirano et al. Cancer Research 2005; 65:1089-1096). The expression of PD-L1 has been correlated with the progression and poor prognosis of certain types of human cancers (Wang et al. European journal of surgical oncology: the journal of the European Society of Surgical Oncology and the British Association of Surgical Oncology 2015; 41:450-456; Cierna et al. Annals of oncology: official journal of the European Society for Medical Oncology/ESMO 2016; 27:300-305; Gandini et al. Critical reviews in oncology/hematology 2016; 100:88-98; Thierauf et al. Melanoma research 2015; 25:503-509; Taube et al. Clinical cancer research: an official journal of the American Association for Cancer Research 2014; 20:5064-5074). During chronic viral infections, PD-L1 is persistently expressed on many tissues, while PD-1 is up-regulated on virus-specific CTLs (Yao et al. Trends in molecular medicine 2006; 12:244-246). Tumor- or virus-induced PD-L1 may utilize multiple mechanisms to facilitate the evasion of host immune surveillance, including T cell anergy, apoptosis, exhaustion, IL-10 production, DC suppression, as well as Treg induction and expansion (Zou et al. Nature reviews Immunology 2008; 8:467-477).

The PD-L1 expression level determined using immunohistochemistry (IHC) has been assessed as a predictive biomarker in clinical trials of PD-1/PD-L1-directed therapy on multiple cancer types, including melanoma, renal cell carcinoma (RCC), non-small cell lung cancer (NSCLC), metastatic colorectal cancer (mCRC), and metastatic castration-resistant prostate cancer (mCRPC). Patients with higher levels of PD-L1 determined by IHC appeared to have superior responses to PD-1/PD-L1-directed therapy. However, PD-L1-negative patients with melanoma can still obtain durable response to anti-PD-1/PD-L1 therapy, while response rates in PD-L1-negative NSCLC patients are rare.

The accuracy of PD-L1 detection by IHC in human tumor specimens is confounded by multiple factors. A multitude of PD-L1 antibodies for IHC detection have been utilized, including 28-8, 22C3, 5H1, MIH1, and 405.9A11. In addition, a number of proprietary companion diagnostics are being developed in this area, such as Ventana SP142 and Ventana SP263 assay. Comparative performance characteristics of these assays are not well known. In addition to the existing issue of heterogeneous PD-L1 expression within the tumor microenvironment, there's also a lack of a clear definition of “positive” PD-L1 staining by IHC in tumor samples. Cut-off points for a positive result could range from >1% to >50%, based on percent tumor cells stained. Furthermore, PD-L1 has limited binding sites for IHC detection antibodies, as it contains only two small hydrophilic regions, which makes immunohistochemical approaches classically used in formalin-fixed, paraffin-embedded (FFPE) specimens less effective. Due to the lack of binding sites on PD-L1, IHC antibodies typically bind PD-L1 at structurally unique sites compared with therapeutic PD-L1 antibodies.

Additionally, PD-L1 is biologically active only when expressed on the cell membrane, either through dynamic IFNγ expression or through constitutive oncogene activation. Oncogene-driven PD-L1 expression represents a histopathologically and biologically distinct entity compared to inflammation driven PD-L1 expression. While the latter occurs focally at sites of IFNγ-mediated immunologic attack, oncogene-driven PD-L1 expression is constitutive and diffuse. IFNγ induced PD-L1 expression represents a dynamic biomarker and is present at sites of active inflammation, and biopsy samples represent a snapshot of the tumor immune microenvironment in space and time. Other factors in the tumor metabolic microenvironment, including hypoxia, can result in PD-L1 upregulation and are dependent on signaling via HIF1α. Smaller tumor biopsies may miss the pertinent tumor-immune interface, or the biopsy may be performed after the biologically relevant PD-L1 overexpression has already taken place. PD-L1 itself is expressed at two potentially clinically relevant immunologic synapses—the tumor/T-cell interface, as well as the APC/T-cell interface. For the tumor/T-cell interface, biopsy capture of the tumor/immune interface is a key determinant in PD-L1 detection by IHC in melanoma. In a study assessing PD-L1 expression in patients with metastatic melanoma, 96% of PD-L1-overexpressing melanomas had lymphocytic infiltrate (TIL), while the remaining 4% of PD-L1-overexpressing lacked TILs, possibly representing oncogene-driven PD-L1 expression. In addition, 22% of PD-L1 negative samples were associated with TIL, indicating alternative mechanisms of tumor immune interference.

The majority of PD-L1 expression occurs at the tumor interface, with immune cells secreting IFNγ, leading to the counterintuitive hypothesis that PD-L1 overexpression may be an initially protective response to successful tumor killing by TILs, which over time becomes co-opted into an immunosuppressive tumor environment. In addition, selection of the appropriate site for biopsy for PD-L1 detection remains enigmatic. While pretreatment FFPE primary tumor samples may be most readily available, these samples may not reflect the overall immunologic state that currently exists in a given patient, particularly if interim treatment has been administered. The absence of PD-L1 expression in a biopsied lesion may not reflect the systemic immunologic landscape, and may not capture the beneficial effect of the therapy at other sites of the disease that are dependent on PD-L1 signaling. In summary, there is an unmet need for accurate and alternative PD-L1 detection agents and methods.

The disclosures of all publications, patents, patent applications and published patent applications referred to herein, including PCT Application No. PCT/CN2018/089672, are hereby incorporated herein by reference in their entirety.

### BRIEF SUMMARY OF THE INVENTION

The present application provides antibody agents, polynucleotides, nucleic acid constructs, vectors, host cells, culture media, kits, methods of determining the distribution of an immune checkpoint in an individual, methods of diagnositing an individual, and methods of treating an individual using the antibody agents.

One aspect of the present application provides an antibody agent comprising an antibody moiety, wherein the antibody moiety is an antibody or an antigen-binding fragment thereof, wherein the antibody or antigen-binding fragment thereof comprises a heavy chain variable region (V_{H}) and a light chain variable region (V_{L}), wherein: a) the V_{H} comprises an HC-CDR1 comprising the amino acid sequence of SEQ ID NO: 41, an HC-CDR2 comprising the amino acid sequence of SEQ ID NO: 42, and an HC-CDR3 comprising the amino acid sequence of SEQ ID NO: 43, or a variant thereof comprising up to a total of about 5 amino acid substitutions in the HC-CDRs; and b) the V_{L} comprises an LC-CDR1 comprising the amino acid sequence of SEQ ID NO: 44, an LC-CDR2 comprising the amino acid sequence of SEQ ID NO: 45, and an LC-CDR3 comprising the amino acid sequence of SEQ ID NO: 46, or a variant thereof comprising up to a total of about 5 amino acid substitutions in the LC-CDRs.

In some embodiments according to any one of the antibody agents described above, the VH comprises an HC-CDR1 comprising the amino acid sequence of SEQ ID NO: 41, an HC-CDR2 comprising the amino acid sequence of SEQ ID NO: 42, and an HC-CDR3 comprising the amino acid sequence of SEQ ID NO: 43; and/or the V_{L} comprises an LC-CDR1 comprising the amino acid sequence of SEQ ID NO: 44, an LC-CDR2 comprising the amino acid sequence of SEQ ID NO: 45, and an LC-CDR3 comprising the amino acid sequence of SEQ ID NO: 46.

In some embodiments according to any one of the antibody agents described above, the V_{H} comprises an amino acid sequence having at least about 80% sequence identity to the amino acid sequence of any one of SEQ ID NOs: 1, 5, 9, 11, and 13; and the V_{L} comprises an amino acid sequence having at least about 80% sequence identity to the amino acid sequence of any one of SEQ ID NOs: 3, 7, 15, 17 and 19. In some embodiments, the antibody moiety comprises an amino acid sequence having at least about 80% sequence identity to the amino acid sequence of SEQ ID NO: 21 or 23.

One aspect of the present application provides an antibody agent an antibody moiety, wherein said antibody moiety is an antibody or an antigen-binding fragment thereof, wherein said antibody or antigen-binding fragment thereof specifically binds to PD-L1 competitively with an anti-PD-L1 antibody comprising a VH and a VL, wherein: a) the V_{H} comprises an HC-CDR1 comprising the amino acid sequence of SEQ ID NO: 41, an HC-CDR2 comprising the amino acid sequence of SEQ ID NO: 42, and an HC-CDR3 comprising the amino acid sequence of SEQ ID NO: 43; and b) the V_{L} comprises an LC-CDR1 comprising the amino acid sequence of SEQ ID NO: 44, an LC-CDR2 comprising the amino acid sequence of SEQ ID NO: 45, and an LC-CDR3 comprising the amino acid sequence of SEQ ID NO: 46.

In some embodiments according to any one of the antibody agents described above, the antibody moiety is selected from the group consisting of a single-chain Fv (scFv), a Fab, a Fab′, a F(ab′)2, an Fv fragment, a disulfide stabilized Fv fragment (dsFv), a (dsFv)₂, a V_{H}H, a Fv-Fc fusion, a scFv-Fc fusion, a scFv-Fv fusion, a diabody, a tribody, and a tetrabody.

In some embodiments according to any one of the antibody agents described above, the antibody moiety has an isotype selected from the group consisting of an IgG, an IgM, an IgA, an IgD, and an IgE.

In some embodiments according to any one of the antibody agents described above, the antibody moiety comprises a scFv fused to an Fc fragment. In some embodiments, the Fc fragment comprises H310A and H435Q mutations, wherein the amino acid positions are based on the Kabat numbering system.

In some embodiments according to any one of the antibody agents described above, the antibody agent further comprises a first conjugation moiety, wherein the first conjugation moiety is capable of being conjugated to a second conjugation moiety in vivo. In some embodiments, the first conjugation moiety comprises a member of a click chemistry pair. In some embodiments, the click chemistry pair is selected from the group consisting of a trans-cyclooctene (TCO)-tetrazine (Tz) pair, an azide-alkyne pair, an alkyne-nitrone pair, an alkene-tetrazole pair, and an isonitrile-tetrazine pair.

One aspect of the present application provides a polynucleotide encoding the antibody moiety of the antibody agent according to any one of the antibody agents described above.

One aspect of the present application provides a nucleic acid construct, comprising the polynucleotide according to any one of the polynucleotides described above, optionally further comprising a promoter in operative connection with the polynucleotide.

One aspect of the present application provides a vector comprising the nucleic acid construct according to any one of the nucleic acid constructs described above.

One aspect of the present application provides a host cell comprising the polynucleotide according to any one of the polynucleotides described above, the nucleic acid construct according to any one of the nucleic acid constructs described above, or the vector according to any one of the vectors described above.

One aspect of the present application provides a culture medium comprising the antibody moiety of the antibody agent according to any one of the antibody agents described above, the polynucleotide according to any one of the polynucleotides described above, the nucleic acid construct according to any one of nucleic acid construct described above, the vector according to any one of the vectors described above, or the host cell according to any one of the host cells described above.

One aspect of the present application provides a method of determining the distribution of an immune checkpoint ligand in an individual, comprising: administering to the individual an effective amount of an antibody agent comprising an antibody moiety and a first conjugation moiety, wherein the antibody moiety specifically binds the immune checkpoint ligand; subsequently administering to the individual an effective amount of a radionuclide compound comprising a radionuclide and a second conjugation moiety, wherein the first conjugation moiety and the second conjugation moiety is conjugated to each other in vivo to provide an imaging agent; and imaging the imaging agent in the individual with a non-invasive imaging technique.

In some embodiments according to any one of the methods described above, the first conjugation moiety and the second conjugation moiety each comprises a member of a click chemistry pair, and are conjugated to each other via click chemistry. In some embodiments, the chemistry pair is selected from the group consisting of a TCO-Tz pair, an azide-alkyne pair, an alkyne-nitrone pair, an alkene-tetrazole pair, and an isonitrile-tetrazine pair. In some embodiments, the first conjugation moiety is a trans-cyclooctene (TCO) and the second conjugation moiety is a tetrazine (Tz), or the first conjugation moiety is a Tz and the second conjugation moiety is a TCO.

In some embodiments according to any one of the methods described above, the radionuclide compound is administered between about 1 hour and about 100 hours after the administration of the antibody agent.

In some embodiments according to any one of the methods described above, the effective amount of the antibody agent is between about 0.1 mg/kg and about 100 mg/kg.

In some embodiments according to any one of the methods described above, the effective amount of the radionuclide is between about 10 uCi and 500 uCi.

In some embodiments according to any one of the methods described above, the imaging is carried out between about 30 minutes and about 24 hours after administration of the radionuclide compound.

In some embodiments according to any one of the methods described above, the method further comprising determining the expression level of the immune checkpoint ligand in a tissue of interest in the individual based on signals emitted by the imaging agent from the tissue.

In some embodiments according to any one of the methods described above, the imaging agent is cleared from the individual within between about 10 minutes and about seven days.

In some embodiments according to any one of the methods described above, the half-life of the antibody agent is between about 10 minutes and about 8 days in serum.

In some embodiments according to any one of the methods described above, the binding between the antibody moiety and the immune checkpoint ligand has a K_{D} between about 9×10⁻¹⁰ M and about 1×10⁻⁸ M.

In some embodiments according to any one of the methods described above, the molecular weight of the antibody moiety is no more than about 400 kDa.

In some embodiments according to any one of the methods described above, the antibody moiety cross-reacts with the immune checkpoint ligand from a non-human mammal.

In some embodiments according to any one of the methods described above, the antibody moiety is humanized.

In some embodiments according to any one of the methods described above, the antibody moiety is stable at acidic pH.

In some embodiments according to any one of the methods described above, the antibody moiety has a melting temperature (Tm) of about 55-70° C.

In some embodiments according to any one of the methods described above, the antibody moiety is selected from the group consisting of a single-chain Fv (scFv), a Fab, a Fab′, a F(ab′)2, an Fv fragment, a disulfide stabilized Fv fragment (dsFv), a (dsFv)₂, a V_{H}H, a Fv-Fc fusion, a scFv-Fc fusion, a scFv-Fv fusion, a diabody, a tribody, and a tetrabody.

In some embodiments according to any one of the methods described above, the antibody moiety comprises an scFv fused to an Fc fragment. In some embodiments, the Fc fragment is a human IgG1 Fc fragment. In some embodiments, the Fc fragment comprises H310A and H435Q mutations, wherein the amino acid positions are based on the Kabat numbering system.

In some embodiments according to any one of the methods described above, the immune checkpoint ligand is PD-L1 or a PD-L1 like ligand.

In some embodiments according to any one of the methods described above, the antibody moiety comprises heavy chain variable region (V_{H}) and a light chain variable region (V_{L}), wherein: a) the V_{H} comprises a heavy chain complementarity determining region 1 (HC-CDR1) comprising the amino acid sequence of SEQ ID NO: 41, an HC-CDR2 comprising the amino acid sequence of SEQ ID NO: 42, and an HC-CDR3 comprising the amino acid sequence of SEQ ID NO: 43, or a variant thereof comprising up to a total of about 5 amino acid substitutions in the HC-CDRs; and b) the V_{L} comprises a light chain complementarity determining region 1 (LC-CDR1) comprising the amino acid sequence of SEQ ID NO: 44, an LC-CDR2 comprising the amino acid sequence of SEQ ID NO: 45, and an LC-CDR3 comprising the amino acid sequence of SEQ ID NO: 46, or a variant thereof comprising up to a total of about 5 amino acid substitutions in the LC-CDRs.

In some embodiments according to any one of the methods described above, the tissue of interest is negative for the immune checkpoint ligand based on an immunohistochemistry (IHC) assay.

In some embodiments according to any one of the methods described above, the tissue of interest has a low expression level of the immune checkpoint ligand.

In some embodiments according to any one of the methods described above, the tissue of interest only expresses the immune checkpoint ligand upon infiltration of immune cells.

In some embodiments according to any one of the methods described above, the method further comprising imaging the individual over a period of time.

In some embodiments according to any one of the methods described above, the radionuclide is selected from the group consisting of ⁶⁴Cu, ¹⁸F, ⁶⁷Ga, ⁶⁸Ga, ¹¹¹In, ¹⁷⁷Lu, ⁹⁰Y, ⁸⁹Zr, ⁶¹Cu, ⁶²Cu, ⁶⁷Cu, ¹⁹F, ⁶⁶Ga, ⁷²Ga, ⁴⁴Sc, ⁴⁷Sc, ⁸⁶Y, ⁸⁸Y and ⁴⁵Ti. In some embodiments, the radionuclide is ⁶⁸Ga.

In some embodiments according to any one of the methods described above, the radionuclide compound comprises a chelating compound that chelates the radionuclide. In some embodiments, the chelating compound is 1,4,7-triazacyclononane-1,4,7-trisacetic acid (NOTA), 1,4,7,10-tetraazacyclododecane-1,4,7,10-tetraacetic acid (DOTA) or a derivative thereof.

In some embodiments according to any one of the methods described above, the non-invasive imaging technique comprises single photon emission computed tomography (SPECT) imaging or positron emission tomography (PET) imaging.

In some embodiments according to any one of the methods described above, the non-invasive imaging technique comprises computed tomography imaging, magnetic resonance imaging, chemical luminescence imaging, or electrochemical luminescence imaging.

In some embodiments according to any one of the methods described above, the antibody agent is administered intravenously, intraperitoneally, intramuscularly, subcutaneously, or orally.

In some embodiments according to any one of the methods described above, the radionuclide compound is administered intravenously, intraperitoneally, intramuscularly, subcutaneously, or orally.

In some embodiments according to any one of the methods described above, the individual has a solid tumor, a hematological malignancy, an infectious disease, autoimmune disease, or metabolic disease.

One aspect of the present application provides a method of diagnosing an individual having a disease or condition, comprising: determining the distribution of an immune checkpoint ligand in the individual using the method according to any one of the methods described above; and diagnosing the individual as positive for the immune checkpoint ligand if signal of the imaging agent is detected at a tissue of interest, or diagnosing the individual as negative for the immune checkpoint ligand if signal of the imaging agent is not detected at a tissue of interest.

One aspect of the present application provides a method of treating an individual having a disease or condition, comprising: diagnosing the individual using the method according to any of the methods described above; and administering to the individual an effective amount of a therapeutic agent targeting the immune checkpoint ligand, if the individual is diagnosed as positive for the immune checkpoint ligand.

One aspect of the present application provides a kit comprising: an antibody agent comprising an antibody moiety and a first conjugation moiety, wherein the antibody moiety specifically binds an immune checkpoint ligand; and a radionuclide compound comprising a radionuclide and a second conjugation moiety, wherein the first conjugation moiety and the second conjugation moiety are capable of conjugating with each other in vivo.

One aspect of the present application provides a computer system comprising: an input unit that receives a request from a user to determine the distribution of an immune checkpoint ligand in an individual; one or more computer processors operatively coupled to the input unit, wherein the one or more computer processors are individually or collectively programmed to: receiving a set of data comprising a signal of an imaging agent at a tissue of interest in an individual, wherein the imaging agent is produced in vivo via a conjugation of a first conjugation moiety and a second conjugation moiety after: an administration of an effective amount of an antibody agent comprising an antibody moiety and the first conjugation moiety into the individual, wherein the antibody moiety specifically binds the immune checkpoint ligand, and a subsequent administration an effective amount of a radionuclide compound comprising a radionuclide and the second conjugation moiety into the individual; presenting the data in a readable manner or generating an analysis of the data.

### BRIEF DESCRIPTION OF THE DRAWINGS

FIGS. 1A and 1B show the binding affinity of anti hPD-L1 monoclonal antibody 5B7 to hPD-L1 protein. FIG. 1A shows histograms demonstrating the binding affinity of anti hPD-L1 monoclonal antibody 5B7 to hPD-L1 protein at different concentrations. FIG. 1B shows the mean fluorescence intensity at the respective concentrations.

FIGS. 2A and 2B show the binding affinity of the anti-hPD-L1 monoclonal antibody to CHO cells expressing hPD-L1 protein or mPD-L1 protein.

FIG. 3 shows the binding affinities of humanized anti-hPD-L1 antibodies to PD-L1 as compared to the parental mouse antibody.

FIG. 4 shows binding affinity and kinetics profiles of humanized antibodies as compared to the parental mouse antibody.

FIG. 5 shows a schematic diagram of the construct design for scFv-HuFc(Wt) and scFv-HuFc(Mt).

FIG. 6 shows the SDS-PAGE results for scFv-HuFc(Wt) and scFv-HuFc(Mt) in both the reduced and non-reduced conditions.

FIG. 7 shows histograms demonstrating the binding affinities of scFv-HuFc(Wt) and scFv-HuFc(Mt) to PD-L1 as compared to that of the parental antibody.

FIG. 8 shows binding affinity and kinetics profile of scFv-HuFc fusion proteins.

FIG. 9 shows serum titers of anti-hPD-L1 antibodies and hIgG following intravenous injections of anti-hPD-L1 scFv-HuFc fusion proteins scFv-hFc Wt and scFv-hFc Mt.

FIG. 10 shows the labeling rate of DOTA-(PEG)₁₀-Tz at 100° C. Region 1 (Rgn 1, from 3.5 to 29.5 nm) represents ⁶⁸Ga-DOTA-Tz. Region 2 (Rgn 2, from 64.2 to 84.1 nm) represents ⁶⁸Ga.

FIG. 11 shows the labeling rate of 2 μL DOTA-(PEG)₁₀-Tz at 100° C. Region 1 (Rgn 1, from 1.7 to 26 nm) represents ⁶⁸Ga-DOTA-Tz. Region 2 (Rgn 2, from 50.3 to 64.2 nm) represents ⁶⁸Ga.

FIG. 12 shows ⁶⁸Ga-Tz radioactivity detected for MC-38 cells or MC38-B7H1 cells under the treatment of ⁶⁸Ga-DOTA-Tz-TCO-FMU-230B.

FIG. 13 shows the ⁶⁸Ga-Tz radioactivity detected for MC-38 cells or MC38-B7H1 cells under the treatment of ⁶⁸Ga-DOTA-Tz-TCO-FMU-220.

FIG. 14 shows in vivo imaging of tumors induced by injections of MC38-PD-L1 cells as described in Example 6IA. Specifically, mice were injected via tail veil with TCO-FMU-220. Three hours later, ⁶⁸Ga-Tz was injected into the tail vein. Mice were then imaged at 60 minutes and 150 minutes after injection.

FIG. 15 shows in vivo imaging of tumors induced by injections of MC38-PD-L1 cells as described in Example 6IB. Specifically, mice were injected via tail veil with TCO-FMU-220. Twenty-four hours later, ⁶⁸Ga-Tz was injected into the tail vein. Mice were then imaged at 60 minutes and 150 minutes after injection.

FIG. 16 shows in vivo imaging of tumors induced by injections of MC38-PD-L1 cells as described in Example 6IC. Specifically, mice were injected via tail veil with TCO-FMU-220. Forty-eight hours later, ⁶⁸Ga-Tz was injected into the tail vein. Mice were then imaged at 60 minutes and 150 minutes after injection.

FIG. 17 shows in vivo imaging of tumors induced by injections of MC38-PD-L1 cells as described in Example 6IIA. Specifically, mice were injected via tail veil with TCO-FMU-230B. Three hours later, ⁶⁸Ga-Tz was injected into the tail vein. Mice were then imaged at 60 minutes and 150 minutes after injection.

FIG. 18 shows in vivo imaging of tumors induced by injections of MC38-PD-L1 cells as described in Example 6IIB. Specifically, mice were injected via tail veil with TCO-FMU-230B. Twenty-four hours later, ⁶⁸Ga-Tz was injected into the tail vein. Mice were then imaged at 60 minutes and 150 minutes after injection.

FIG. 19 shows in vivo imaging of tumors induced by injections of MC38-PD-L1 cells as described in Example 6IIC. Specifically, mice were injected via tail veil with TCO-FMU-230B. Forty-eight hours later, ⁶⁸Ga-Tz was injected into the tail vein. Mice were then imaged at 60 minutes and 150 minutes after injection.

### DETAILED DESCRIPTION OF THE INVENTION

The present application in one aspect provides methods for detection of an immune checkpoint ligand in an individual as well as compositions, kits, computer systems and articles of manufacture useful for such methods. The methods described herein involve use of a) an antibody agent comprising an antibody moiety and a first conjugation moiety, wherein the antibody moiety specifically binds an immune checkpoint ligand, such as PD-L1 or a PD-L1 like ligand, and b) a radionuclide compound comprising a radionuclide and a second conjugation moiety, wherein the first conjugation moiety and the second conjugation moiety can be conjugated to each other in vivo to provide a targeted imaging agent.

The methods as described herein provides the advantage of avoiding unwanted clinical complications by allowing pre-targeting the individual with the antibody agent that binds to the target immune checkpoint ligand and then subsequently administering radionuclide compound. The antibody agent and radionuclide compound each comprises a conjugation moiety, which can specifically be conjugated (e.g., via click chemistry) to each other in vivo to provide an imaging agent. For example, the first conjugation moiety is a trans-cyclooctene (TCO) and the second conjugation moiety is a tetrazine (Tz), or the first conjugation moiety is a Tz and the second conjugation moiety is a TCO.

The antibody moieties in some embodiments are engineered to increase thermal stability. In some embodiments, the antibody moieties have a relatively high molecular weight (e.g., higher than that of scFv used in an antibody/radionucleotide conjugation). In some embodiments, the radionucleotide is short-lived. The subject matter disclosed in the present application thus are advantageous over prior imaging methods involving administration of antibody/radionucleotide conjugate method, which are not suitable for bulky antibody moieties and short-lived radionucleotides.

Accordingly, one aspect of the present application provides a method of determining the distribution of an immune checkpoint ligand (such as PD-L1 or a PD-L1 like ligand) in an individual, comprising: (a) administering to the individual an antibody agent comprising an antibody moiety and a first conjugation moiety, wherein the antibody moiety specifically binds the immune checkpoint ligand; (b) subsequently administering to the individual a label compound (e.g., a radionuclide compound) comprising a label (e.g., radionuclide) and a second conjugation moiety, wherein the first conjugation moiety and the second conjugation moiety are conjugated to each other in vivo to provide an imaging agent; and (c) imaging the imaging agent in the individual with a non-invasive imaging technique.

Another aspect of the present application provides an antibody agent comprising an antibody moiety and a first conjugation moiety, wherein the antibody moiety specifically binds an immune checkpoint ligand (such as PD-L1 or a PD-L1 like ligand), and wherein the conjugation moiety can be conjugated to a second conjugation moiety in vivo.

Another aspect of the present application provides an anti-PD-L1 antibody agent comprising: a V_{H} comprising a HC-CDR1, a HC-CDR2, and a HC-CDR3 of SEQ ID NO: 1; and a V_{L} comprising a LC-CDR1, a LC-CDR2, and a LC-CDR3 of SEQ ID NO: 3.

Another aspect of the present application provides a label compound (e.g., a radionuclide compound) comprising a label (e.g., a radionuclide) and a second conjugation moiety, and wherein the conjugation moiety can be conjugated to a first conjugation moiety in vivo.

Another aspect of the present application provides a polynucleotide, a nucleic acid construct, a vector, a host cell, a culture medium as described herein.

Also provided are compositions, kits and articles of manufacture, comprising the antibody agents (such as anti-PD-L1 antibody agents) and/or label compound (e.g., radionuclide compound) described herein, methods of making thereof, and methods of diagnosing or treating an individual having a disease or condition (such as cancer, infectious disease, autoimmune disease or metabolic disease).

### I. Definitions

As used herein, “immune system checkpoints,” or “immune checkpoints” refer to inhibitory pathways in the immune system that generally act to maintain self-tolerance or modulate the duration and amplitude of physiological immune responses to minimize collateral tissue damage. Stimulatory checkpoint molecules are molecules, such as proteins, that stimulate or positively regulate the immune system. Inhibitory checkpoint molecules are molecules, such as proteins, that inhibit or negatively regulate the immune system. Immune system checkpoint molecules include, but are not limited to, cytotoxic T-lymphocyte antigen 4 (CTLA-4), programmed cell death 1 protein (PD-1), PD-L1, PD-L2, lymphocyte activation gene 3 (LAG3), B7-1, B7-H3, B7-H4, T cell membrane protein 3 (TIM3), B- and T-lymphocyte attenuator (BTLA), V-domain immunoglobulin (Ig)-containing suppressor of T-cell activation (VISTA), Killer-cell immunoglobulin-like receptor (KIR), and A2A adenosine receptor (A2aR).

“Immune checkpoint receptors” are immune checkpoint molecules that are expressed on immune cells, such as T cells.

As used herein, the term “immune checkpoint ligand” refers to a naturally-occurring or non-naturally occurring ligand that is specifically recognized by an immune checkpoint receptor. Naturally occurring immune checkpoint ligands are immune checkpoint molecules that may be expressed by diseased tissue, such as tumor cells, infected cells, or inflamed tissue, which can regulate immune cells that express immune checkpoint receptors that specifically recognize the immune checkpoint ligands. Non-naturally occurring immune checkpoint ligands include synthetic and recombinant molecules, such as therapeutic inhibitors, ligands, and antibodies of immune checkpoint receptors. Non-naturally occurring immune checkpoint ligands may be introduced to the individual, e.g., by administration to the individual. An immune checkpoint ligand can inhibit an immune checkpoint by stimulating the activity of a stimulatory checkpoint receptor, or inhibiting the activity of an inhibitory checkpoint receptor in the pathway. Exemplary naturally-occurring immune checkpoint ligands include, but are not limited to, PD-L1, PD-L2, B7-H3 (also known as CD276), galectin-9, CD80, CD86 and ICOSL. In some embodiments, the immune checkpoint ligand is PD-L1. In some embodiments, the immune checkpoint ligand is a PD-L1 like ligand. “PD-L1 like ligand” refers to a naturally occurring or non-naturally occurring ligand of PD-1.

The term “antibody” is used in its broadest sense and encompasses various antibody structures, including but not limited to monoclonal antibodies, polyclonal antibodies, multispecific antibodies (e.g., bispecific antibodies), full-length antibodies and antigen-binding fragments thereof, so long as they exhibit the desired antigen-binding activity. The term “antibody moiety” refers to a full-length antibody or an antigen-binding fragment thereof.

A full-length antibody comprises two heavy chains and two light chains. The variable regions of the light and heavy chains are responsible for antigen binding. The variable domains of the heavy chain and light chain may be referred to as “V_{H}” and “V_{L}”, respectively. The variable regions in both chains generally contain three highly variable loops called the complementarity determining regions (CDRs) (light chain (LC) CDRs including LC-CDR1, LC-CDR2, and LC-CDR3, heavy chain (HC) CDRs including HC-CDR1, HC-CDR2, and HC-CDR3). CDR boundaries for the antibodies and antigen-binding fragments disclosed herein may be defined or identified by the conventions of Kabat, Chothia, or Al-Lazikani (Al-Lazikani 1997; Chothia 1985; Chothia 1987; Chothia 1989; Kabat 1987; Kabat 1991). The three CDRs of the heavy or light chains are interposed between flanking stretches known as framework regions (FRs), which are more highly conserved than the CDRs and form a scaffold to support the hypervariable loops. The constant regions of the heavy and light chains are not involved in antigen binding, but exhibit various effector functions. Antibodies are assigned to classes based on the amino acid sequence of the constant region of their heavy chain. The five major classes or isotypes of antibodies are IgA, IgD, IgE, IgG, and IgM, which are characterized by the presence of α, δ, ε, γ, and μ heavy chains, respectively. Several of the major antibody classes are divided into subclasses such as IgG1 (γ1 heavy chain), IgG2 (γ2 heavy chain), IgG3 (γ3 heavy chain), IgG4 (γ4 heavy chain), IgA1 (α1 heavy chain), or IgA2 (α2 heavy chain).

The term “antigen-binding fragment” as used herein refers to an antibody fragment including, for example, a diabody, a Fab, a Fab′, a F(ab′)2, an Fv fragment, a disulfide stabilized Fv fragment (dsFv), a (dsFv)2, a bispecific dsFv (dsFv-dsFv′), a disulfide stabilized diabody (ds diabody), a single-chain Fv (scFv), an scFv dimer (bivalent diabody), a multispecific antibody formed from a portion of an antibody comprising one or more CDRs, a camelized single domain antibody, a nanobody, a domain antibody, a bivalent domain antibody, or any other antibody fragment that binds to an antigen but does not comprise a complete antibody structure. An antigen-binding fragment is capable of binding to the same antigen to which the parent antibody or a parent antibody fragment (e.g., a parent scFv) binds. In some embodiments, an antigen-binding fragment may comprise one or more CDRs from a particular human antibody grafted to a framework region from one or more different human antibodies.

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the heavy and light chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Single-chain Fv,” also abbreviated as “sFv” or “scFv,” are antibody fragments that comprise the V_{H} and V_{L} antibody domains connected into a single polypeptide chain. In some embodiments, the scFv polypeptide further comprises a polypeptide linker between the V_{H} and V_{L} domains which enables the scFv to form the desired structure for antigen binding. For a review of scFv, see Plückthun in The Pharmacology of Monoclonal Antibodies, vol. 113, Rosenburg and Moore eds., Springer-Verlag, New York, pp. 269-315 (1994).

The term “diabodies” refers to small antibody fragments prepared by constructing scFv fragments (see preceding paragraph) typically with short linkers (such as about 5 to about 10 residues) between the V_{H} and V_{L} domains such that inter-chain but not intra-chain pairing of the V domains is achieved, resulting in a bivalent fragment, i.e., fragment having two antigen-binding sites. Bispecific diabodies are heterodimers of two “crossover” scFv fragments in which the V_{H} and V_{L} domains of the two antibodies are present on different polypeptide chains. Diabodies are described more fully in, for example, EP 404,097; WO 93/11161; and Hollinger et al., Proc. Natl. Acad. Sci. USA, 90:6444-6448 (1993).

As used herein, the term “CDR” or “complementarity determining region” is intended to mean the non-contiguous antigen combining sites found within the variable region of both heavy and light chain polypeptides. These particular regions have been described by Kabat et al., J. Biol. Chem. 252:6609-6616 (1977); Kabat et al., U.S. Dept. of Health and Human Services, “Sequences of proteins of immunological interest” (1991); Chothia et al., J. Mol. Biol. 196:901-917 (1987); Al-Lazikani B. et al., J. Mol. Biol., 273: 927-948 (1997); MacCallum et al., J. Mol. Biol. 262:732-745 (1996); Abhinandan and Martin, Mol. Immunol., 45: 3832-3839 (2008); Lefranc M. P. et al, Dev. Comp. Immunol., 27: 55-77 (2003); and Honegger and Plückthun, J. Mol. Biol., 309:657-670 (2001), where the definitions include overlapping or subsets of amino acid residues when compared against each other. Nevertheless, application of either definition to refer to a CDR of an antibody or grafted antibodies or variants thereof is intended to be within the scope of the term as defined and used herein. The amino acid residues which encompass the CDRs as defined by each of the above cited references are set forth below in Table 1 as a comparison. CDR prediction algorithms and interfaces are known in the art, including, for example, Abhinandan and Martin, Mol. Immunol., 45: 3832-3839 (2008); Ehrenmann F. et al., Nucleic Acids Res., 38: D301-D307 (2010); and Adolf-Bryfogle J. et al., Nucleic Acids Res., 43: D432-D438 (2015). The contents of the references cited in this paragraph are incorporated herein by reference in their entireties for use in the present invention and for possible inclusion in one or more claims herein.

TABLE 1
CDR DEFINITIONS

| | Kabat¹ | Chothia² | MacCallum³ | IMGT⁴ | AHo⁵ |
|---|---|---|---|---|---|
| V_{H} CDR1 | 31-35 | 26-32 | 30-35 | 27-38 | 25-40 |
| V_{H} CDR2 | 50-65 | 53-55 | 47-58 | 56-65 | 58-77 |
| V_{H} CDR3 | 95-102 | 96-101 | 93-101 | 105-117 | 109-137 |
| V_{L} CDR1 | 24-34 | 26-32 | 30-36 | 27-38 | 25-40 |
| V_{L} CDR2 | 50-56 | 50-52 | 46-55 | 56-65 | 58-77 |
| V_{L} CDR3 | 89-97 | 91-96 | 89-96 | 105-117 | 109-137 |

¹Residue numbering follows the nomenclature of Kabat et al., supra
²Residue numbering follows the nomenclature of Chothia et al., supra
³Residue numbering follows the nomenclature of MacCallum et al., supra
⁴Residue numbering follows the nomenclature of Lefranc et al., supra
⁵Residue numbering follows the nomenclature of Honegger and Plückthun, supra

The expression “variable-domain residue-numbering as in Kabat” or “amino-acid-position numbering as in Kabat,” and variations thereof, refers to the numbering system used for heavy-chain variable domains or light-chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy-chain variable domain may include a single amino acid insert (residue 52a according to Kabat) after residue 52 of H2 and inserted residues (e.g. residues 82a, 82b, and 82c, etc. according to Kabat) after heavy-chain FR residue 82. The Kabat numbering of residues may be determined for a given antibody by alignment at regions of homology of the sequence of the antibody with a “standard” Kabat numbered sequence.

Unless indicated otherwise herein, the numbering of the residues in an immunoglobulin heavy chain is that of the EU index as in Kabat et al., supra. The “EU index as in Kabat” refers to the residue numbering of the human IgG1 EU antibody.

“Framework” or “FR” residues are those variable-domain residues other than the CDR residues as herein defined.

The term “chimeric antibodies” refer to antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit a biological activity of this invention (see U.S. Pat. No. 4,816,567; and Morrison et al., Proc. Natl. Acad. Sci. USA, 81:6851-6855 (1984)).

The term “semi-synthetic” in reference to an antibody or antibody moiety means that the antibody or antibody moiety has one or more naturally occurring sequences and one or more non-naturally occurring (i.e., synthetic) sequences.

“Humanized” forms of non-human (e.g., rodent) antibodies are chimeric antibodies that contain minimal sequence derived from the non-human antibody. For the most part, humanized antibodies are human immunoglobulins (recipient antibody) in which residues from a hypervariable region (HVR) of the recipient are replaced by residues from a hypervariable region of a non-human species (donor antibody) such as mouse, rat, rabbit or non-human primate having the desired antibody specificity, affinity, and capability. In some instances, framework region (FR) residues of the human immunoglobulin are replaced by corresponding non-human residues. Furthermore, humanized antibodies can comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

A “human antibody” is an antibody that possesses an amino-acid sequence corresponding to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol., 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA, 103:3557-3562 (2006) regarding human antibodies generated via a human B-cell hybridoma technology.

“Percent (%) amino acid sequence identity” or “homology” with respect to the polypeptide and antibody sequences identified herein is defined as the percentage of amino acid residues in a candidate sequence that are identical with the amino acid residues in the polypeptide being compared, after aligning the sequences considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, Megalign (DNASTAR), or MUSCLE software. Those skilled in the art can determine appropriate parameters for measuring alignment, including any algorithms needed to achieve maximal alignment over the full-length of the sequences being compared. For purposes herein, however, % amino acid sequence identity values are generated using the sequence comparison computer program MUSCLE (Edgar, R. C., Nucleic Acids Research 32(5):1792-1797, 2004; Edgar, R. C., BMC Bioinformatics 5(1):113, 2004).

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared times 100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

The term “constant domain” refers to the portion of an immunoglobulin molecule having a more conserved amino acid sequence relative to the other portion of the immunoglobulin, the variable domain, which contains the antigen-binding site. The constant domain contains the C_{H}1, C_{H}2 and C_{H}3 domains (collectively, C_{H}) of the heavy chain and the CHL (or C_{L}) domain of the light chain.

The “light chains” of antibodies (immunoglobulins) from any mammalian species can be assigned to one of two clearly distinct types, called kappa (“κ”) and lambda (“λ”), based on the amino acid sequences of their constant domains.

The “CH1 domain” of a human IgG Fc region (also referred to as “C1” of “H1” domain) usually extends from about amino acid 118 to about amino acid 215 (EU numbering system).

“Hinge region” is generally defined as stretching from Glu216 to Pro230 of human IgG1 (Burton, Molec. Immunol. 22:161-206 (1985)). Hinge regions of other IgG isotypes may be aligned with the IgG1 sequence by placing the first and last cysteine residues forming inter-heavy chain S—S bonds in the same positions.

The “CH2 domain” of a human IgG Fc region (also referred to as “C2” of “H2” domain) usually extends from about amino acid 231 to about amino acid 340. The CH2 domain is unique in that it is not closely paired with another domain. Rather, two N-linked branched carbohydrate chains are interposed between the two CH2 domains of an intact native IgG molecule. It has been speculated that the carbohydrate may provide a substitute for the domain-domain pairing and help stabilize the CH2 domain. Burton, Molec Immunol. 22:161-206 (1985).

The “CH3 domain” (also referred to as “C2” or “H3” domain) comprises the stretch of residues C-terminal to a CH2 domain in an Fc region (i.e. from about amino acid residue 341 to the C-terminal end of an antibody sequence, typically at amino acid residue 446 or 447 of an IgG).

The term “Fc region” or “fragment crystallizable region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. Suitable native-sequence Fc regions for use in the antibodies described herein include human IgG1, IgG2 (IgG2A, IgG2B), IgG3 and IgG4.

“Fc receptor” or “FcR” describes a receptor that binds the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors, FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (See M. Daëron, Annu. Rev. Immunol. 15:203-234 (1997). FcRs are reviewed in Ravetch and Kinet, Annu. Rev. Immunol. 9: 457-92 (1991); Capel et al., Immunomethods 4: 25-34 (1994); and de Haas et al., J. Lab. Clin. Med. 126: 330-41 (1995). Other FcRs, including those to be identified in the future, are encompassed by the term “FcR” herein.

The term “epitope” as used herein refers to the specific group of atoms or amino acids on an antigen to which an antibody or antibody moiety binds. Two antibodies or antibody moieties may bind the same epitope within an antigen if they exhibit competitive binding for the antigen.

As used herein, a first antibody moiety “competes” for binding to a target antigen with a second antibody moiety when the first antibody moiety inhibits the target antigen binding of the second antibody moiety by at least about 50% (such as at least about any one of 55%, 60%, 65%, 70%, 75%, 80%, 85%, 90%, 95%, 98% or 99%) in the presence of an equimolar concentration of the first antibody moiety, or vice versa. A high throughput process for “binning” antibodies based upon their cross-competition is described in PCT Publication No. WO 03/48731.

As use herein, the terms “specifically binds,” “specifically recognizing,” and “is specific for” refer to measurable and reproducible interactions, such as binding between a target and an antibody or antibody moiety, which is determinative of the presence of the target in the presence of a heterogeneous population of molecules, including biological molecules. For example, an antibody or antibody moiety that specifically recognizes a target (which can be an epitope) is an antibody or antibody moiety that binds this target with greater affinity, avidity, more readily, and/or with greater duration than its bindings to other targets. In some embodiments, the extent of binding of an antibody to an unrelated target is less than about 10% of the binding of the antibody to the target as measured, e.g., by a radioimmunoassay (RIA). In some embodiments, an antibody that specifically binds a target has a dissociation constant (K_{D}) of ≤10⁻⁵ M, ≤10⁻⁶ M, ≤10⁻⁷ M, ≤10⁻⁸ M, ≤10⁻⁹ M, ≤10⁻¹⁰ M, ≤10⁻¹¹ M, or ≤10⁻¹² M. In some embodiments, an antibody specifically binds an epitope on a protein that is conserved among the protein from different species. In some embodiments, specific binding can include, but does not require exclusive binding. Binding specificity of the antibody or antigen-binding domain can be determined experimentally by methods known in the art. Such methods comprise, but are not limited to Western blots, ELISA-, RIA-, ECL-, IRMA-, EIA-, BIACORE™-tests and peptide scans.

An “isolated” antibody (or construct) is one that has been identified, separated and/or recovered from a component of its production environment (e.g., natural or recombinant). Preferably, the isolated polypeptide is free of association with all other components from its production environment.

An “isolated” nucleic acid molecule encoding a construct, antibody, or antigen-binding fragment thereof described herein is a nucleic acid molecule that is identified and separated from at least one contaminant nucleic acid molecule with which it is ordinarily associated in the environment in which it was produced. Preferably, the isolated nucleic acid is free of association with all components associated with the production environment. The isolated nucleic acid molecules encoding the polypeptides and antibodies described herein is in a form other than in the form or setting in which it is found in nature. Isolated nucleic acid molecules therefore are distinguished from nucleic acid encoding the polypeptides and antibodies described herein existing naturally in cells. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

As used herein, “treatment” or “treating” is an approach for obtaining beneficial or desired results, including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delay or slowing the progression of the disease, ameliorating the disease state, providing a remission (partial or total) of the disease, decreasing the dose of one or more other medications required to treat the disease, delaying the progression of the disease, increasing or improving the quality of life, increasing weight gain, and/or prolonging survival. Also encompassed by “treatment” is a reduction of pathological consequence of cancer (such as, for example, tumor volume). The methods of the invention contemplate any one or more of these aspects of treatment.

The term “effective amount” refers to the amount of an agent that is sufficient to effect beneficial or desired results. The term also applies to a dose that will provide an image for detection by any one of the imaging methods described herein. The specific dose may vary depending on one or more of the particular agent chosen, the dosing regimen to be followed, whether it is administered in combination with other compounds, timing of administration, the tissue to be imaged, and the physical delivery system in which it is carried.

The terms “subject,” “individual,” and “patient” are used interchangeably herein to refer to a mammal, including, but not limited to, human, bovine, horse, feline, canine, rodent, or primate. In some embodiments, the individual is a human.

It is understood that embodiments of the invention described herein include “consisting” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat cancer of type X means the method is used to treat cancer of types other than X.

The term “about X-Y” used herein has the same meaning as “about X to about Y.”

As used herein and in the appended claims, the singular forms “a,” “or,” and “the” include plural referents unless the context clearly dictates otherwise.

“Conjugated” used herein refers to specific association of two conjugation moieties, which can be covalent or non-covalent.

### II. Methods of Imaging

One aspect of the present application provides a method of determining the distribution and/or expression level of an immune checkpoint ligand in an individual using a) an effective amount of an antibody agent comprising an antibody moiety and a first conjugation moiety, and b) a effective amount of a label compound (e.g., radionuclide compound) comprising a label (e.g., radionuclide) and a second conjugation moiety, wherein the first conjugation moiety and the second conjugation moiety are conjugated to each other in vivo to provide an imaging agent. In some embodiments, the method further comprises imaging the imaging agent in the individual with a non-invasive imaging technique.

In some embodiments, there is provided a method of determining the distribution of an immune checkpoint ligand in an individual, comprising: a) administering to the individual an effective amount of an antibody agent comprising an antibody moiety and a first conjugation moiety, wherein the antibody moiety specifically binds the immune checkpoint ligand; b) subsequently administering to the individual an effective amount of a radionuclide compound comprising a radionuclide and a second conjugation moiety, wherein the first conjugation moiety and the second conjugation moiety are conjugated to each other in vivo to provide an imaging agent; and c) imaging the imaging agent in the individual with a non-invasive imaging technique. In some embodiments, there is provided a method of determining the distribution of an immune checkpoint ligand in an individual, comprising administering to the individual an effective amount of an antibody agent comprising an antibody moiety and a first conjugation moiety, wherein the antibody moiety specifically binds the immune checkpoint ligand, wherein the individual is to be administered with an effective amount of a radionuclide compound comprising a radionuclide and a second conjugation moiety, wherein the first conjugation moiety and the second conjugation moiety are conjugated to each other in vivo to provide an imaging agent. In some embodiments, there is provided a method of determining the distribution of an immune checkpoint ligand in an individual who has been administered with an effective amount of an antibody agent comprising an antibody moiety and a first conjugation moiety, wherein the antibody moiety specifically binds the immune checkpoint ligand, comprising administering to the individual an effective amount of a radionuclide compound comprising a radionuclide and a second conjugation moiety, wherein the first conjugation moiety and the second conjugation moiety are conjugated to each other in vivo to provide an imaging agent.

In some embodiments, there is provided a method of determining the distribution of an PD-L1 or PD-L1 like ligand in an individual, comprising: a) administering to the individual an effective amount of an antibody agent comprising an antibody moiety and a first conjugation moiety, wherein the antibody moiety specifically binds an PD-L1 or PD-L1 like ligand; b) subsequently administering to the individual an effective amount of a radionuclide compound comprising a radionuclide and a second conjugation moiety, wherein the first conjugation moiety and the second conjugation moiety are conjugated to each other in vivo to provide an imaging agent; and c) imaging the imaging agent in the individual with a non-invasive imaging technique. In some embodiments, the radionuclide is selected from the group consisting of ⁶⁴Cu, ¹⁸F, ⁶⁷Ga, ⁶⁸Ga, ¹¹¹In, ¹⁷⁷Lu, ⁹⁰Y, ⁸⁹Zr, ⁶¹Cu, ⁶²Cu, ⁶⁷Cu, ¹⁹F, ⁶⁶Ga, ⁷²Ga, ⁴⁴Sc, ⁴⁷Sc, ⁸⁶Y, ⁸⁸Y and ⁴⁵Ti. In some embodiments, the radionuclide is ⁶⁸Ga. In some embodiments, the first conjugation moiety and the second conjugation moiety each comprises a member of a click chemistry pair, and are conjugated to each other via click chemistry. In some embodiments, the first conjugation moiety is a trans-cyclooctene (TCO) and the second conjugation moiety is a tetrazine (Tz), or the first conjugation moiety is a Tz and the second conjugation moiety is a TCO. In some embodiments, the radionuclide compound is administered immediately after the administration of the antibody agent. In some embodiments, the radionuclide compound is administered between about 1 hour and about 100 hours after the administration of the antibody agent. In some embodiments, the antibody moiety comprises an scFv fused to an Fc fragment. In some embodiments, the Fc fragment comprises H310A and H435Q mutations, wherein the amino acid positions are based on the Kabat numbering system. In some embodiments, the radionuclide compound and/or the antibody agent comprises a chelating compound that chelates the radionuclide. In some embodiments, the chelating compound is 1,4,7-triazacyclononane-1,4,7-trisacetic acid (NOTA), 1,4,7,10-tetraazacyclododecane-1,4,7,10-tetraacetic acid (DOTA) or a derivative thereof. In some embodiments, the individual has a solid tumor, a hematological malignancy, an infectious disease, autoimmune disease, or metabolic disease.

In some embodiments, there is provided a method of determining the distribution of an PD-L1 or PD-L1 like ligand in an individual, comprising: a) administering to the individual an effective amount of an antibody agent comprising an antibody moiety and a first conjugation moiety, wherein the antibody moiety specifically binds an PD-L1 or PD-L1 like ligand; b) subsequently administering to the individual an effective amount of a radionuclide compound comprising ⁶⁸Ga and a second conjugation moiety, wherein the first conjugation moiety and the second conjugation moiety are conjugated to each other in vivo to provide an imaging agent; and c) imaging the imaging agent in the individual with a non-invasive imaging technique. In some embodiments, the first conjugation moiety and the second conjugation moiety each comprises a member of a click chemistry pair, and are conjugated to each other via click chemistry. In some embodiments, the first conjugation moiety is a trans-cyclooctene (TCO) and the second conjugation moiety is a tetrazine (Tz), or the first conjugation moiety is a Tz and the second conjugation moiety is a TCO. In some embodiments, the radionuclide compound is administered immediately after the administration of the antibody agent. In some embodiments, the radionuclide compound is administered between about 1 hour to about 100 hours after the administration of the antibody agent). In some embodiments, the antibody moiety comprises an scFv fused to an Fc fragment. In some embodiments, the Fc fragment comprises H310A and H435Q mutations, wherein the amino acid positions are based on the Kabat numbering system. In some embodiments, the radionuclide compound and/or the antibody agent comprises a chelating compound that chelates the radionuclide. In some embodiments, the chelating compound is 1,4,7-triazacyclononane-1,4,7-trisacetic acid (NOTA), 1,4,7,10-tetraazacyclododecane-1,4,7,10-tetraacetic acid (DOTA) or a derivative thereof. In some embodiments, the individual has a solid tumor, a hematological malignancy, an infectious disease, autoimmune disease, or metabolic disease.

In some embodiments, there is provided a method of determining the distribution of an PD-L1 or PD-L1 like ligand in an individual, comprising: a) administering to the individual an effective amount of an antibody agent comprising an antibody moiety and a first conjugation moiety, wherein the antibody moiety specifically binds an PD-L1 or PD-L1 like ligand; b) subsequently administering to the individual an effective amount of a radionuclide compound comprising ⁶⁸Ga and a second conjugation moiety, wherein the first conjugation moiety and the second conjugation moiety are conjugated to each other in vivo to provide an imaging agent, wherein the first conjugation moiety is a trans-cyclooctene (TCO) and the second conjugation moiety is a tetrazine (Tz), or the first conjugation moiety is a Tz and the second conjugation moiety is a TCO; and c) imaging the imaging agent in the individual with a non-invasive imaging technique. In some embodiments, the radionuclide compound is administered immediately after the administration of the antibody agent. In some embodiments, the radionuclide compound is administered between about 1 hour to about 100 hours after the administration of the antibody agent). In some embodiments, the antibody moiety comprises an scFv fused to an Fc fragment. In some embodiments, the Fc fragment comprises H310A and H435Q mutations, wherein the amino acid positions are based on the Kabat numbering system. In some embodiments, the radionuclide compound and/or the antibody agent comprises a chelating compound that chelates the radionuclide. In some embodiments, the chelating compound is 1,4,7-triazacyclononane-1,4,7-trisacetic acid (NOTA), 1,4,7,10-tetraazacyclododecane-1,4,7,10-tetraacetic acid (DOTA) or a derivative thereof. In some embodiments, the individual has a solid tumor, a hematological malignancy, an infectious disease, autoimmune disease, or metabolic disease.

In some embodiments, there is provided a method of determining the distribution of an immune checkpoint ligand in an individual, comprising: a) administering to the individual an effective amount of an antibody agent comprising an antibody moiety and a first conjugation moiety, wherein the antibody moiety comprises a VH comprises an HC-CDR1 comprising the amino acid sequence of SEQ ID NO: 41, an HC-CDR2 comprising the amino acid sequence of SEQ ID NO: 42, and an HC-CDR3 comprising the amino acid sequence of SEQ ID NO: 43 and a V_{L} comprises an LC-CDR1 comprising the amino acid sequence of SEQ ID NO: 44, an LC-CDR2 comprising the amino acid sequence of SEQ ID NO: 45, and an LC-CDR3 comprising the amino acid sequence of SEQ ID NO: 46; b) subsequently administering to the individual an effective amount of a radionuclide compound comprising a radionuclide and a second conjugation moiety, wherein the first conjugation moiety and the second conjugation moiety are conjugated to each other in vivo to provide an imaging agent; and c) imaging the imaging agent in the individual with a non-invasive imaging technique. In some embodiments, the radionuclide is selected from the group consisting of ⁶⁴Cu, ¹⁸F, ⁶⁷Ga, ⁶⁸Ga, ¹¹¹In, ¹⁷⁷Lu, ⁹⁰Y, ⁸⁹Zr, ⁶¹Cu, ⁶²Cu, ⁶⁷Cu, ¹⁹F, ⁶⁶Ga, ⁷²Ga, ⁴⁴Sc, ⁴⁷Sc, ⁸⁶Y, ⁸⁸Y and ⁴⁵Ti. In some embodiments, the radionuclide is ⁶⁸Ga. In some embodiments, the first conjugation moiety and the second conjugation moiety each comprises a member of a click chemistry pair, and are conjugated to each other via click chemistry. In some embodiments, the first conjugation moiety is a trans-cyclooctene (TCO) and the second conjugation moiety is a tetrazine (Tz), or the first conjugation moiety is a Tz and the second conjugation moiety is a TCO. In some embodiments, the radionuclide compound is administered immediately after the administration of the antibody agent. In some embodiments, the radionuclide compound is administered between about 1 hour to about 100 hours after the administration of the antibody agent). In some embodiments, the antibody moiety comprises an scFv fused to an Fc fragment. In some embodiments, the Fc fragment comprises H310A and H435Q mutations, wherein the amino acid positions are based on the Kabat numbering system. In some embodiments, the radionuclide compound and/or the antibody agent comprises a chelating compound that chelates the radionuclide. In some embodiments, the chelating compound is 1,4,7-triazacyclononane-1,4,7-trisacetic acid (NOTA), 1,4,7,10-tetraazacyclododecane-1,4,7,10-tetraacetic acid (DOTA) or a derivative thereof. In some embodiments, the individual has a solid tumor, a hematological malignancy, an infectious disease, autoimmune disease, or metabolic disease.

In some embodiments, there is provided a method of determining the distribution of an immune checkpoint ligand in an individual, comprising: a) administering to the individual an effective amount of an antibody agent comprising an antibody moiety and a first conjugation moiety, wherein the antibody moiety comprises a VH comprises an HC-CDR1 comprising the amino acid sequence of SEQ ID NO: 41, an HC-CDR2 comprising the amino acid sequence of SEQ ID NO: 42, and an HC-CDR3 comprising the amino acid sequence of SEQ ID NO: 43 and a V_{L} comprises an LC-CDR1 comprising the amino acid sequence of SEQ ID NO: 44, an LC-CDR2 comprising the amino acid sequence of SEQ ID NO: 45, and an LC-CDR3 comprising the amino acid sequence of SEQ ID NO: 46; b) subsequently administering to the individual an effective amount of a radionuclide compound comprising a radionuclide and a second conjugation moiety, wherein the first conjugation moiety and the second conjugation moiety are conjugated to each other in vivo to provide an imaging agent, wherein the first conjugation moiety is a trans-cyclooctene (TCO) and the second conjugation moiety is a tetrazine (Tz), or the first conjugation moiety is a Tz and the second conjugation moiety is a TCO; and c) imaging the imaging agent in the individual with a non-invasive imaging technique. In some embodiments, the radionuclide is selected from the group consisting of ⁶⁴Cu, ¹⁸F, ⁶⁷Ga, ⁶⁸Ga, ¹¹¹In, ¹⁷⁷Lu, ⁹⁰Y, ⁸⁹Zr, ⁶¹Cu, ⁶²Cu, ⁶⁷Cu, ¹⁹F, ⁶⁶Ga, ⁷²Ga, ⁴⁴Sc, ⁴⁷Sc, ⁸⁶Y, ⁸⁸Y and ⁴⁵Ti. In some embodiments, the radionuclide is ⁶⁸Ga. In some embodiments, the radionuclide compound is administered immediately after the administration of the antibody agent. In some embodiments, the radionuclide compound is administered between about 1 hour to about 100 hours after the administration of the antibody agent). In some embodiments, the antibody moiety comprises an scFv fused to an Fc fragment. In some embodiments, the Fc fragment comprises H310A and H435Q mutations, wherein the amino acid positions are based on the Kabat numbering system. In some embodiments, the radionuclide compound and/or the antibody agent comprises a chelating compound that chelates the radionuclide. In some embodiments, the chelating compound is 1,4,7-triazacyclononane-1,4,7-trisacetic acid (NOTA), 1,4,7,10-tetraazacyclododecane-1,4,7,10-tetraacetic acid (DOTA) or a derivative thereof. In some embodiments, the individual has a solid tumor, a hematological malignancy, an infectious disease, autoimmune disease, or metabolic disease.

In some embodiments, there is provided a method of determining the distribution of an immune checkpoint ligand in an individual, comprising: a) administering to the individual an effective amount of an antibody agent comprising an antibody moiety and a first conjugation moiety, wherein the antibody moiety comprises a VH comprises an HC-CDR1 comprising the amino acid sequence of SEQ ID NO: 41, an HC-CDR2 comprising the amino acid sequence of SEQ ID NO: 42, and an HC-CDR3 comprising the amino acid sequence of SEQ ID NO: 43 and a V_{L} comprises an LC-CDR1 comprising the amino acid sequence of SEQ ID NO: 44, an LC-CDR2 comprising the amino acid sequence of SEQ ID NO: 45, and an LC-CDR3 comprising the amino acid sequence of SEQ ID NO: 46; b) subsequently administering to the individual an effective amount of a radionuclide compound comprising ⁶⁸Ga and a second conjugation moiety, wherein the first conjugation moiety and the second conjugation moiety are conjugated to each other in vivo to provide an imaging agent, wherein the first conjugation moiety is a trans-cyclooctene (TCO) and the second conjugation moiety is a tetrazine (Tz), or the first conjugation moiety is a Tz and the second conjugation moiety is a TCO; and c) imaging the imaging agent in the individual with a non-invasive imaging technique. In some embodiments, the radionuclide compound is administered immediately after the administration of the antibody agent. In some embodiments, the radionuclide compound is administered between about 1 hour to about 100 hours after the administration of the antibody agent). In some embodiments, the antibody moiety comprises an scFv fused to an Fc fragment. In some embodiments, the Fc fragment comprises H310A and H435Q mutations, wherein the amino acid positions are based on the Kabat numbering system. In some embodiments, the radionuclide compound and/or the antibody agent comprises a chelating compound that chelates the radionuclide. In some embodiments, the chelating compound is 1,4,7-triazacyclononane-1,4,7-trisacetic acid (NOTA), 1,4,7,10-tetraazacyclododecane-1,4,7,10-tetraacetic acid (DOTA) or a derivative thereof. In some embodiments, the individual has a solid tumor, a hematological malignancy, an infectious disease, autoimmune disease, or metabolic disease.

In some embodiments, there is provided a method of determining the distribution of an immune checkpoint ligand in an individual, comprising: a) administering to the individual an effective amount of an antibody agent comprising an antibody moiety and a first conjugation moiety, wherein the antibody moiety comprises a heavy chain variable region (V_{H}) comprising an amino acid sequence having at least about 80% sequence identity to the amino acid sequence of any one of SEQ ID NOs: 1, 5, 9, 11, and 13; and a variable light chain region (V_{L}) comprising an amino acid sequence having at least about 80% sequence identity to the amino acid sequence of any one of SEQ ID NOs: 3, 7, 15, 17 and 19; b) subsequently administering to the individual an effective amount of a radionuclide compound comprising a radionuclide (such as ⁶⁸Ga) and a second conjugation moiety, wherein the first conjugation moiety and the second conjugation moiety are conjugated to each other in vivo to provide an imaging agent, wherein the first conjugation moiety is a trans-cyclooctene (TCO) and the second conjugation moiety is a tetrazine (Tz), or the first conjugation moiety is a Tz and the second conjugation moiety is a TCO; and c) imaging the imaging agent in the individual with a non-invasive imaging technique. In some embodiments, the radionuclide compound is administered immediately after the administration of the antibody agent. In some embodiments, the radionuclide compound is administered between about 1 hour and about 100 hours after the administration of the antibody agent). In some embodiments, the antibody moiety comprises an scFv fused to an Fc fragment. In some embodiments, the Fc fragment comprises H310A and H435Q mutations, wherein the amino acid positions are based on the Kabat numbering system. In some embodiments, the radionuclide compound and/or the antibody agent comprises a chelating compound that chelates the radionuclide. In some embodiments, the chelating compound is 1,4,7-triazacyclononane-1,4,7-trisacetic acid (NOTA), 1,4,7,10-tetraazacyclododecane-1,4,7,10-tetraacetic acid (DOTA) or a derivative thereof. In some embodiments, the individual has a solid tumor, a hematological malignancy, an infectious disease, autoimmune disease, or metabolic disease.

In some embodiments, there is provided a method of determining the distribution of an immune checkpoint ligand in an individual, comprising: a) administering to the individual an effective amount of an antibody agent comprising an antibody moiety and a first conjugation moiety, wherein the antibody moiety comprises an anti-PD-L1 scFv comprises the amino acid sequence of any one of SEQ ID NOs: 25, 27, 29, 31, 33, 35, 37 and 39, or a variant thereof having at least about 80% (such as at least about any one of 80%, 85%, 90%, 95%, 96%, 97%, 98%, or 99%) sequence identity to the amino acid sequence of any one of SEQ ID NOs: 25, 27, 29, 31, 33, 35, 37 and 39; b) subsequently administering to the individual an effective amount of a radionuclide compound comprising a radionuclide (such as ⁶⁸Ga.) and a second conjugation moiety, wherein the first conjugation moiety and the second conjugation moiety are conjugated to each other in vivo to provide an imaging agent, wherein the first conjugation moiety is a trans-cyclooctene (TCO) and the second conjugation moiety is a tetrazine (Tz), or the first conjugation moiety is a Tz and the second conjugation moiety is a TCO; and c) imaging the imaging agent in the individual with a non-invasive imaging technique. In some embodiments, the radionuclide compound is administered immediately after the administration of the antibody agent. In some embodiments, the radionuclide compound is administered between about 1 hour and about 100 hours after the administration of the antibody agent). In some embodiments, the antibody moiety comprises an scFv fused to an Fc fragment. In some embodiments, the Fc fragment comprises H310A and H435Q mutations, wherein the amino acid positions are based on the Kabat numbering system. In some embodiments, the radionuclide compound and/or the antibody agent comprises a chelating compound that chelates the radionuclide. In some embodiments, the chelating compound is 1,4,7-triazacyclononane-1,4,7-trisacetic acid (NOTA), 1,4,7,10-tetraazacyclododecane-1,4,7,10-tetraacetic acid (DOTA) or a derivative thereof. In some embodiments, the individual has a solid tumor, a hematological malignancy, an infectious disease, autoimmune disease, or metabolic disease.

In some embodiments, the label compound (e.g., radionuclide compound) is administered immediately after the administration of the antibody agent. In some embodiments, the label compound (e.g., radionuclide compound) is administered between about 1 hour and about 100 hours (e.g., about 2 hours and about 72 hours, about 3 hours and about 48 hours) after the administration of the antibody agent. In some embodiments, the label compound (e.g., radionuclide compound) is administered between about 1 hour and about 2 hours, about 2 hours and about 4 hours, about 4 hours and about 8 hours, about 8 hours and about 12 hours, about 12 hours and about 18 hours, about 18 hours and about 24 hours, about 24 hours and about 36 hours, about 36 hours and about 48 hours, about 48 hours and about 72 hours, about 72 hours and about 96 hours. In some embodiments, the label compound (e.g., radionuclide compound) is administered within about 1, 2, 3, 4, 6, 8, 12, 16, 20, 24, 36, 48, 72, or 96 hours after the administration of the antibody agent. In some embodiments, the label compound (e.g., radionuclide compound) is administered at least about 1, 2, 3, 4, 6, 8, 12, 16, 20, 24, 36, 48, 72, or 96 hours after the administration of the antibody agent.

The antibody agent and/or label compound (e.g., radionuclide compound) described herein can be administered to an individual (such as human) via various routes, including, for example, intravenous, intra-arterial, intraperitoneal, intrapulmonary, oral, inhalation, intravesicular, intramuscular, intra-tracheal, subcutaneous, intraocular, intrathecal, transmucosal, and transdermal.

In some embodiments, the effective amount of the antibody agent is from about 0.1 mg/kg to about 100 mg/kg. In some embodiments, the effective amount of the antibody agent is from about 0.1 mg/kg to about 1 mg/kg, about 1 mg/kg to about 10 mg/kg, about 10 mg/kg to about 25 mg/kg, about 25 mg/kg to about 50 mg/kg, about 50 mg/kg to about 75 mg/kg, about 75 mg/kg to about 100 mg/kg. In some embodiments, the effective amount of the antibody agent is no less than about 0.1 mg/kg, 0.5 mg/kg, 1 mg/kg, 5 mg/kg, 10 mg/kg, 25 mg/kg, 50 mg/kg, 75 mg/kg, 100 mg/kg. In some embodiments, the effective amount of the antibody agent is no more than about 0.1 mg/kg, 0.5 mg/kg, 1 mg/kg, 5 mg/kg, 10 mg/kg, 25 mg/kg, 50 mg/kg, 75 mg/kg, 100 mg/kg.

In some embodiments, the effective amount of the radionuclide is from about 10 uCi to about 500 uCi. In some embodiments, the effective amount of the radionuclide is from about 10 uCi to about 25 uCi, about 25 uCi to about 50 uCi, about 50 uCi to about 75 uCi, about 75 uCi to about 100 uCi, about 100 uCi to about 125 uCi, about 125 uCi to about 150 uCi, about 150 uCi to about 175 uCi, about 175 uCi to about 200 uCi, about 200 uCi to about 250 uCi, about 250 uCi to about 300 uCi, about 300 uCi to about 350 uCi, about 350 uCi to about 400 uCi, about 400 uCi to 450 uCi, about 450 uCi to about 500 uCi. In some embodiments, the effective amount of the radionuclide is no less than about 10, 25, 50, 75, 100, 125, 150, 175, 200, 250, 300, 350, 400, 450, 500 uCi. In some embodiments, the effective amount of the radionuclide is no more than about 10, 25, 50, 75, 100, 125, 150, 175, 200, 250, 300, 350, 400, 450, 500 uCi.

In some embodiments, the imaging agent, once formed is cleared from the individual within about 10 minutes to about seven days. In some embodiments, the imaging agent is cleared within about 1 hour, 3 hours, 6 hours, 12 hours, or 24 hours. In some embodiments, the imaging agent is cleared within about 2, 3, 4, 5, 6 or 7 days.

In some embodiments, the antibody agent is cleared from the individual within about 10 minutes to about fourteen days after administration. In some embodiments, the antibody agent is cleared from the individual within about 1 hour, 3 hours, 6 hours, 12 hours, or 24 hours after administration. In some embodiments, the antibody agent is cleared within about 2, 3, 4, 5, 6, 7, 8, 9, 10, 12, or 14 days after administration. In some embodiments, the antibody agent is cleared from the individual at least about 1 hour, 2 hours, 3 hours, 6 hours, 12 hours, or 24 hours after the administration. In some embodiments, the antibody agent is cleared from the individual at least about 1, 2, 3, 4, 5, 6, 7, 8, 9, 10, 12, or 14 days after administration.

In some embodiments, the label compound is cleared from the individual within about 10 minutes to about seven days after administration. In some embodiments, the label compound is cleared from the individual within about 1, 2, 3, 4, 5, 6, 7, 8, 9, 10, 11, 12, 15, 18, 21, or 24 hours after administration. In some embodiments, the label compound agent is cleared within about 2, 3, 4, 5, 6, or 7 days. In some embodiments, the label compound is cleared before the clearance of the antibody agent. In some embodiments, the label compound is cleared at least about 3, 6, 9, 12, 18 hours before the clearance of the antibody agent. In some embodiments, the label compound is cleared at least about 1, 2, 3, 4, 5, 6, or 7 days before the clearance of the antibody agent.

In some embodiments, the imaging is carried out between about 30 minutes and about 1 hour, about 1 hours to about 2 hours, about 2 hours to about 3 hours, about 3 hours to about 4 hours, about 4 hours to about 6 hours, about 6 hours to about 9 hours, about 9 hours to about 12 hours, about 12 hours to about 18 hours, or about 18 hours to about 24 hours after the administration of the label compound (e.g., radionuclide compound). In some embodiments, the imaging is carried out at least about 30 minutes, 1 hours, 2 hours, 3 hours, 4 hours, 6 hours, 9 hours, 12 hours, 18 hours after the administration of the label compound (e.g., radionuclide compound). In some embodiments, the imaging is carried out within 30 minutes, 1 hours, 2 hours, 3 hours, 4 hours, 6 hours, 9 hours, 12 hours, 18 hours after the administration of the label compound (e.g., radionuclide compound).

In some embodiments, the method further comprises administering to the individual an antibody moiety not linked to a first conjugation moiety prior to the administration of the antibody agent.

In some embodiments, the individual has a solid tumor. In some embodiments, the individual has a hematological malignancy. In some embodiments, the individual has an infectious disease, autoimmune disease, or metabolic disease.

In some embodiments, the individual is a mammal (e.g., a human).

The methods described herein can utilize methods of imaging using labeled polypeptides well known in the art, and any such known methods. See, for example, Srivastava (ed.), Radiolabeled Monoclonal Antibodies for Imaging and Therapy (Plenum Press 1988), Chase, “Medical Applications of Radioisotopes,” in Remington's Pharmaceutical Sciences, 18th Edition, Gennaro et al. (eds.), pp. 624-652 (Mack Publishing Co., 1990), and Brown, “Clinical Use of Monoclonal Antibodies,” in Biotechnology and Pharmacy 227-49, Pezzuto et al. (eds.) (Chapman & Hall 1993). In some embodiments, the non-invasive imaging technique uses positron-emitting radionuclides (PET isotopes), such as with an energy of about 511 keV, such as ¹⁸F, ⁶⁸Ga, ⁶⁴Cu, and ¹²⁴I. Such radionuclides may be imaged by well-known PET scanning techniques. See, also, U.S. Pat. Nos. 6,953,567; 9,884,131 and international patent application publication No. WO2016149188A1, and Kim H Y. et al., (2018) PLoS ONE 13(3): e0192821, which are incorporated herein by reference.

In some embodiments, the non-invasive imaging technique comprises single photon emission computed tomography (SPECT) imaging. In some embodiments, the non-invasive imaging technique comprises positron emission tomography (PET) imaging. In some embodiments, SPEC or PET imaging is combined with one or more other non-invasive imaging method, which may or may not be based on the signals from the imaging agent. For example, PET may be combined with computed tomography (CT) imaging, magnetic resonance imaging (MRI), chemical luminescence imaging, or electrochemical luminescence imaging.

The imaging methods described herein are suitable for detecting immune checkpoint ligands at low, moderate, or high expression levels. In some embodiments, the imaging method provides dynamic information on the expression level and distribution of the immune checkpoint ligand. In some embodiments, the imaging method is capable of detecting the immune checkpoint ligand in situations that might be challenging for other methods of detection, such as immunohistochemistry (IHC). For example, in some embodiments, the tissue of interest is negative for the immune checkpoint ligand based on an immunohistochemistry (IHC) assay or another assay. Molecular assays that may be used for detecting the presence or absence of an immune checkpoint ligand include, but are not limited to, polymerase chain reaction (PCR)-based assays, next-generation sequencing (NGS) assays, hybridization assays, and ELISA. In some embodiments, the tissue of interest has a low expression level of the immune checkpoint ligand. In some embodiments, the tissue of interest only expresses the immune checkpoint ligand upon infiltration of immune cells.

The imaging agent may be administered to the individual using any suitable dosage and routes of administration. The route of administration is in accordance with known and accepted methods, such as by single or multiple bolus or infusion over a period of time in a suitable manner, e.g., injection or infusion by subcutaneous, intravenous, intraperitoneal, intramuscular, intra-arterial, intralesional, intraarticular, intratumoral, or oral routes. The determination of the appropriate dosage or route of administration is well within the skill of an ordinary artisan. Animal experiments provide reliable guidance for the determination of effective doses for human diagnostic applications. Interspecies scaling of effective doses can be performed following the principles laid down by Mordenti, J. and Chappell, W. “The Use of Interspecies Scaling in Toxicokinetics,” In Toxicokinetics and New Drug Development, Yacobi et al., Eds, Pergamon Press, New York 1989, pp. 42-46.

In some embodiments, the method further comprises imaging the imaging agent at a tissue of interest in the same individual prior to the administration of the antibody agent and/or the label compound. In some embodiments, the methods further comprises comparing the result of the imaging to a result obtained from a prior imaging of the same imaging agent at a tissue of interest in the same individual or a control individual prior to the administration of the antibody agent and/or the label compound. In some embodiments, the prior imaging is carried out within about 100 hours, for example, about 48 hours, 24 hours, 12 hours, 6 hours, 2 hours or immediately prior to the administration of the antibody agent and/or the label compound. In some embodiments, the methods further comprise analyzing the two sets of the results, for example, in a computer system as described herein.

### Diagnosis and Treatment

The methods described herein are useful for diagnosis and as a companion diagnostic method for treatment of a variety of diseases and conditions that are associated with abnormal immune response. In some embodiments, the disease or condition is associated with immune deficiency. In some embodiments, the disease or condition is cancer, infectious disease, autoimmune disease, or a metabolic disease.

In some embodiments, there is provided a method of diagnosing an individual having a disease or condition, comprising: (a) administering to the individual an antibody agent comprising an antibody moiety and a first conjugation moiety, wherein the antibody moiety specifically binds the immune checkpoint ligand; (b) subsequently administering to the individual a label compound (e.g., a radionuclide compound) comprising a label (e.g., radionuclide) and a second conjugation moiety, wherein the first conjugation moiety and the second conjugation moiety are conjugated to each other in vivo to provide an imaging agent; and (c) diagnosing the individual as positive for the immune checkpoint ligand if signal of the imaging agent is detected at a tissue of interest, or diagnosing the individual as negative for the immune checkpoint ligand if signal of the imaging agent is not detected at a tissue of interest. In some embodiments, the first conjugation moiety and the second conjugation moiety are conjugated to each other via click chemistry. In some embodiments, the first conjugation moiety is a trans-cyclooctene (TCO) and the second conjugation moiety is a tetrazine (Tz), or the first conjugation moiety is a Tz and the second conjugation moiety is a TCO. In some embodiments, the radionuclide compound is administered immediately after the administration of the antibody agent. In some embodiments, the radionuclide compound is administered between about 1 hour and about 100 hours after the administration of the antibody agent. In some embodiments, the imaging is carried out between about 30 minutes and about 24 hours after administration of the radionuclide compound. In some embodiments, the disease or condition is cancer, infection, autoimmune disease, or metabolic disease. In some embodiments, the immune checkpoint ligand is PD-L1. In some embodiments, the immune checkpoint ligand is a PD-L1 like ligand.

In some embodiments, there is provided a method of diagnosing an individual having a disease or condition, comprising: (a) administering to an individual an antibody agent comprising an antibody moiety and a first conjugation moiety, wherein the antibody moiety specifically binds the immune checkpoint ligand; (b) subsequently administering to the individual a label compound (e.g., a radionuclide compound) comprising a label (e.g., radionuclide) and a second conjugation moiety, wherein the first conjugation moiety and the second conjugation moiety are conjugated to each other in vivo to provide an imaging agent; (c) imaging the imaging agent in the individual with a non-invasive imaging technique; and (d) diagnosing the individual as positive for the immune checkpoint ligand if signal of the imaging agent is detected at a tissue of interest, or diagnosing the individual as negative for the immune checkpoint ligand if signal of the imaging agent is not detected at a tissue of interest. In some embodiments, the first conjugation moiety and the second conjugation moiety are conjugated to each other via click chemistry. In some embodiments, the first conjugation moiety is a trans-cyclooctene (TCO) and the second conjugation moiety is a tetrazine (Tz), or the first conjugation moiety is a Tz and the second conjugation moiety is a TCO. In some embodiments, the radionuclide compound is administered immediately after the administration of the antibody agent. In some embodiments, the radionuclide compound is administered between about 1 hour and about 100 hours after the administration of the antibody agent. In some embodiments, the imaging is carried out between about 30 minutes and about 24 hours after administration of the radionuclide compound. In some embodiments, the antibody agent is administered intravenously, intraperitoneally, intramuscularly, subcutaneously, or orally. In some embodiments, the radionuclide compound is administered intravenously, intraperitoneally, intramuscularly, subcutaneously, or orally. In some embodiments, the method further comprises administering to the individual an antibody moiety not linked to a first conjugation moiety prior to the administration of the antibody agent. In some embodiments, the method further comprises determining the expression level of the immune checkpoint ligand in a tissue of interest in the individual based on signals emitted by the imaging agent from the tissue. In some embodiments, the non-invasive imaging technique comprises single photon emission computed tomography (SPECT) imaging or positron emission tomography (PET) imaging. In some embodiments, the non-invasive imaging technique comprises or further comprises computed tomography imaging, magnetic resonance imaging, chemical luminescence imaging, or electrochemical luminescence imaging. In some embodiments, the method comprises imaging the individual over a period of time. In some embodiments, the immune checkpoint ligand is selected from the group consisting of PD-L1, PD-L2, B7-H3, galectin-9, CD80, CD86 and ICOSL. In some embodiments, the radionuclide is selected from the group consisting of ⁶⁶⁴Cu, ¹⁸F, ⁶⁷Ga, ⁶⁸Ga, ¹¹¹In, ¹⁷⁷Lu, ⁹⁰Y, ⁸⁹Zr, ⁶¹Cu, ⁶²Cu, ⁶⁷Cu, ¹⁹F, ⁶⁶Ga, ⁷²Ga, ⁴⁴Sc, ⁴⁷Sc, ⁸⁶Y, ⁸⁸Y and ⁴⁵Ti. In some embodiments, the radionuclide is ⁶⁸Ga. In some embodiments, the radionuclide compound comprises a chelating compound that chelates the radionuclide. In some embodiments, the chelating compound is NOTA, DOTA or derivatives thereof. In some embodiments, the antibody moiety has a half-life of about 10 minutes to about 8 days (such as about any one of about 10 minutes to about 2 hours, about 1 hour to about 4 hours, about 4 hours to about 8 hours, about 8 hours to about 12 hours, about 12 hours to about 24 hours, about 24 hours to about 48 hours, about 2 days to about 5 days, and about 5 days to about 8 days.) in serum. In some embodiments, the antibody moiety is no more than about 400 kDa (such as no more than about 350 kDa, 300 kDa, 250 kDa, 200 kDa, 150 kDa). In some embodiments, the antibody moiety is at least about 80, 100, 150, 200, 250, 300, 350, 400, 450, or 500 kDa. In some embodiments, the antibody moiety is about 80-150, 150-250, 250-350, 350-450, or more than 450 kDa. In some embodiments, the binding between the antibody moiety and the immune checkpoint ligand has a K_{D} between about 9×10⁻¹⁰ M and about 1×10⁻⁸ M (such as between about 9×10⁻¹⁰ and 1×10⁻⁹, about 1×10⁻⁹ and 2×10⁻⁹, about 2×10⁻¹⁰ and 5×10⁻⁹, or about 5×10⁻¹⁰ and 1×10⁻⁸) with the immune checkpoint ligand. In some embodiments, the antibody moiety cross-reacts with the immune checkpoint ligand from a different species (e.g., a non-human mammal, e.g., mouse, rat or monkey). In some embodiments, the antibody moiety is humanized. In some embodiments, the antibody moiety is stable at acidic pH (e.g., at a pH lower than about 6.5, 6.0, 5.5, or 5.0). In some embodiments, the antibody moiety has a melting temperature (Tm) of about 55-70° C. (such as about any one of 55-60, 60-65, or 65-70° C.). In some embodiments, the antibody moiety is selected from the group consisting of a single-chain Fv (scFv), a diabody, a Fab, a Fab′, a F(ab′)₂, an Fv fragment, a disulfide stabilized Fv fragment (dsFv), a (dsFv)₂, and a V_{H}H. In some embodiments, the disease or condition is cancer, infection, autoimmune disease, or metabolic disease. In some embodiments, the immune checkpoint ligand is PD-L1. In some embodiments, the immune checkpoint ligand is a PD-L1 like ligand. In some embodiments, the antibody moiety is an scFv. In some embodiments, the antibody moiety is an scFv fused to an Fc fragment (such as a human IgG1 Fc).

In some embodiments, there is provided a method of diagnosing an individual having a disease or condition, comprising: (a) administering to an individual an antibody agent comprising an anti-PD-L1 antibody moiety and a first conjugation moiety; (b) subsequently administering to the individual a label compound (e.g., a radionuclide compound) comprising a label (e.g., radionuclide) and a second conjugation moiety, wherein the first conjugation moiety and the second conjugation moiety are conjugated to each other in vivo to provide an imaging agent; (c) imaging the imaging agent in the individual with a non-invasive imaging technique; and (d) diagnosing the individual as positive for PD-L1 if signal of the imaging agent is detected at a tissue of interest, or diagnosing the individual as negative for the immune checkpoint ligand if signal of the imaging agent is not detected at a tissue of interest; wherein the anti-PD-L1 antibody moiety comprises: a V_{H} comprising a HC-CDR1 comprising the amino acid sequence of SEQ ID NO: 41, a HC-CDR2 comprising the amino acid sequence of SEQ ID NO: 42, and a HC-CDR3 comprising the amino acid sequence of SEQ ID NO: 43, or a variant thereof comprising up to about 5 amino acid substitutions; and a V_{L} comprising a LC-CDR1 comprising the amino acid sequence of SEQ ID NO: 44, a LC-CDR2 comprising the amino acid sequence of SEQ ID NO: 45, and a LC-CDR3 comprising the amino acid sequence of SEQ ID NO: 46, or a variant thereof comprising up to about 5 amino acid substitutions. In some embodiments, the method further comprises determining the expression level of PD-L1 in a tissue of interest in the individual based on signals emitted by the imaging agent from the tissue. In some embodiments, the first conjugation moiety and the second conjugation moiety are conjugated to each other via click chemistry. In some embodiments, the first conjugation moiety is a trans-cyclooctene (TCO) and the second conjugation moiety is a tetrazine (Tz), or the first conjugation moiety is a Tz and the second conjugation moiety is a TCO. In some embodiments, the radionuclide compound is administered immediately after the administration of the antibody agent. In some embodiments, the radionuclide compound is administered between about 1 hour and about 100 hours after the administration of the antibody agent. In some embodiments, the imaging is carried out between about 30 minutes and about 24 hours after administration of the radionuclide compound. In some embodiments, the antibody agent is administered intravenously, intraperitoneally, intramuscularly, subcutaneously, or orally. In some embodiments, the radionuclide compound is administered intravenously, intraperitoneally, intramuscularly, subcutaneously, or orally. In some embodiments, the method further comprises administering to the individual an antibody moiety not linked to a first conjugation moiety prior to the administration of the antibody agent. In some embodiments, the non-invasive imaging technique comprises single photon emission computed tomography (SPECT) imaging or positron emission tomography (PET) imaging. In some embodiments, the non-invasive imaging technique comprises or further comprises computed tomography imaging, magnetic resonance imaging, chemical luminescence imaging, or electrochemical luminescence imaging. In some embodiments, the method comprises imaging the individual over a period of time. In some embodiments, the anti-PD-L1 antibody moiety comprises: a V_{H} comprising the amino acid sequence of any one of SEQ ID NOs: 1, 5, 9, 11, and 13, or a variant thereof having at least about 80% (such as at least about any one of 80%, 85%, 90%, 95%, 96%, 97%, 98%, or 99%) sequence identity to the amino acid sequence of any one of SEQ ID NOs: 1, 5, 9, 11, and 13; and a V_{L} comprising the amino acid sequence of any one of SEQ ID NOs: 3, 7, 15, 17 and 19, or a variant thereof having at least about 80% (such as at least about any one of 80%, 85%, 90%, 95%, 96%, 97%, 98%, or 99%) sequence identity to the amino acid sequence of any one of SEQ ID NOs: 3, 7, 15, 17 and 19. In some embodiments, the anti-PD-L1 antibody moiety is humanized. In some embodiments, the radionuclide is selected from the group consisting of ⁶⁴Cu, ¹⁸F, ⁶⁷Ga, ⁶⁸Ga, ¹¹¹In, ¹⁷⁷Lu, ⁹⁰Y, ⁸⁹Zr, ⁶¹Cu, ⁶²Cu, ⁶⁷Cu, ¹⁹F, ⁶⁶Ga, ⁷²Ga, ⁴⁴Sc, ⁴⁷Sc, ⁸⁶Y, ⁸⁸Y and ⁴⁵Ti. In some embodiments, the radionuclide is ⁶⁸Ga. In some embodiments, the radionuclide compound comprises a chelating compound that chelates the radionuclide. In some embodiments, the chelating compound is NOTA, DOTA or derivatives thereof. In some embodiments, the disease or condition is cancer, infection, autoimmune disease, or metabolic disease. In some embodiments, the antibody moiety is an scFv. In some embodiments, the antibody moiety is an scFv fused to an Fc fragment (such as a human IgG1 Fc). In some embodiments, the scFv comprises one or more (such as 1, 2, 3, or more) engineered disulfide bonds. In some embodiments, the scFv comprises a first engineered cysteine residue at position 44 of V_{H} and a second engineered cysteine residue at position 100 of V_{L}, and/or a first engineered cysteine residue at position 105 of V_{H} and a second engineered cysteine residue at position 43 of V_{L}, wherein the first engineered cysteine residue and the second engineered cysteine residue form a disulfide bond, and wherein the amino acid positions are based on the Kabat numbering system. In some embodiments, the anti-PD-L1 antibody moiety comprises the amino acid sequence of any one of SEQ ID NOs: 25, 27, 29, 31, 33, 35, 37 and 39, or a variant thereof having at least about 80% (such as at least about any one of 80%, 85%, 90%, 95%, 96%, 97%, 98%, or 99%) sequence identity to the amino acid sequence of any one of SEQ ID NOs: 25, 27, 29, 31, 33, 35, 37 and 39.

In some embodiments, there is provided a method of treating an individual having a disease or condition, comprising: (a) diagnosing the individual using any method of diagnosis described herein; and (b) administering to the individual an effective amount of a therapeutic agent targeting the immune checkpoint ligand or receptor thereof, if the individual is diagnosed as positive for the immune checkpoint ligand. In some embodiments, the therapeutic agent is an inhibitor of the immune checkpoint ligand or receptor thereof. In some embodiments, the therapeutic agent is a radiolabeled molecule specifically binding the immune checkpoint ligand or receptor thereof. In some embodiments, the disease or condition is cancer, infection, autoimmune disease, or metabolic disease. In some embodiments, the immune checkpoint ligand is PD-L1. In some embodiments, the immune checkpoint ligand is a PD-L1 like ligand.

In some embodiments, there is provided a method of treating an individual having a disease or condition, comprising: (a) administering to an individual an antibody agent comprising an antibody moiety and a first conjugation moiety, wherein the antibody moiety specifically binds the immune checkpoint ligand; (b) subsequently administering to the individual a label compound (e.g., a radionuclide compound) comprising a label (e.g., radionuclide) and a second conjugation moiety, wherein the first conjugation moiety and the second conjugation moiety are conjugated to each other in vivo to provide an imaging agent; (c) imaging the imaging agent in the individual with a non-invasive imaging technique; (d) diagnosing the individual as positive for the immune checkpoint ligand if signal of the imaging agent is detected at a tissue of interest, or diagnosing the individual as negative for the immune checkpoint ligand if signal of the imaging agent is not detected at a tissue of interest; and (e) administering to the individual an effective amount of a therapeutic agent targeting the immune checkpoint ligand or receptor thereof (e.g., an inhibitor of the immune checkpoint ligand or receptor thereof, or a radiolabeled molecule specifically binding the immune checkpoint ligand or receptor thereof), if the individual is diagnosed as positive for the immune checkpoint ligand. In some embodiments, the method further comprises determining the expression level of the immune checkpoint ligand in a tissue of interest in the individual based on signals emitted by the imaging agent from the tissue. In some embodiments, the first conjugation moiety and the second conjugation moiety are conjugated to each other via click chemistry. In some embodiments, the first conjugation moiety is a trans-cyclooctene (TCO) and the second conjugation moiety is a tetrazine (Tz), or the first conjugation moiety is a Tz and the second conjugation moiety is a TCO. In some embodiments, the radionuclide compound is administered immediately after the administration of the antibody agent. In some embodiments, the radionuclide compound is administered between about 1 hour and about 100 hours after the administration of the antibody agent. In some embodiments, the imaging is carried out between about 30 minutes and about 24 hours after administration of the radionuclide compound. In some embodiments, the antibody agent is administered intravenously, intraperitoneally, intramuscularly, subcutaneously, or orally. In some embodiments, the radionuclide compound is administered intravenously, intraperitoneally, intramuscularly, subcutaneously, or orally. In some embodiments, the method further comprises administering to the individual an antibody moiety not linked to a first conjugation moiety prior to the administration of the antibody agent. In some embodiments, the non-invasive imaging technique comprises single photon emission computed tomography (SPECT) imaging or positron emission tomography (PET) imaging. In some embodiments, the non-invasive imaging technique comprises or further comprises computed tomography imaging, magnetic resonance imaging, chemical luminescence imaging, or electrochemical luminescence imaging. In some embodiments, the method comprises imaging the individual over a period of time. In some embodiments, the immune checkpoint ligand is selected from the group consisting of PD-L1, PD-L2, B7-H3, galectin-9, CD80, CD86 and ICOSL. In some embodiments, the radionuclide is selected from the group consisting of ⁶⁴Cu, ¹⁸F, ⁶⁷Ga, ⁶⁸Ga, ¹¹¹In, ¹⁷⁷Lu, ⁹⁰Y, ⁸⁹Zr, ⁶¹Cu, ⁶²Cu, ⁶⁷Cu, ¹⁹F, ⁶⁶Ga, ⁷²Ga, ⁴⁴Sc, ⁴⁷Sc, ⁸⁶Y, ⁸⁸Y and ⁴⁵Ti. In some embodiments, the radionuclide is ⁶⁸Ga. In some embodiments, the radionuclide compound comprises a chelating compound that chelates the radionuclide. In some embodiments, the chelating compound is NOTA, DOTA or derivatives thereof. In some embodiments, the antibody moiety has a half-life of about 10 minutes to about 8 days (such as about any one of about 10 minutes to about 2 hours, about 1 hour to about 4 hours, about 4 hours to about 8 hours, about 8 hours to about 12 hours, about 12 hours to about 24 hours, about 24 hours to about 48 hours, about 2 days to about 5 days, and about 5 days to about 8 days.) in serum. In some embodiments, the antibody moiety is no more than about 400 kDa (such as no more than about 350 kDa, 300 kDa, 250 kDa, 200 kDa, 150 kDa). In some embodiments, the antibody moiety is at least about 80, 100, 150, 200, 250, 300, 350, 400, 450, or 500 kDa. In some embodiments, the antibody moiety is about 80-150, 150-250, 250-350, 350-450, or more than 450 kDa. In some embodiments, the binding between the antibody moiety and the immune checkpoint ligand has a K_{D} from about 9×10⁻¹⁰ M to about 1×10⁻⁸ M (such as about 9×10⁻¹⁰ to 1×10⁻⁹, about 1×10⁻⁹ to 2×10⁻⁹, about 2×10⁻¹⁰ to 5×10⁻⁹, or about 5×10⁻¹⁰ to 1×10⁻⁸) with the immune checkpoint ligand. In some embodiments, the antibody moiety cross-reacts with the immune checkpoint ligand from a non-human mammal (e.g., mouse, rat or monkey). In some embodiments, the antibody moiety is humanized. In some embodiments, the antibody moiety is stable at acidic pH (e.g., at a pH lower than about 6.5, 6.0, 5.5, or 5.0). In some embodiments, the antibody moiety has a melting temperature (Tm) of about 55-70° C. (such as about any one of 55-60, 60-65, or 65-70° C.). In some embodiments, the antibody moiety is selected from the group consisting of a single-chain Fv (scFv), a diabody, a Fab, a Fab′, a F(ab′)₂, an Fv fragment, a disulfide stabilized Fv fragment (dsFv), a (dsFv)₂, and a V_{H}H. In some embodiments, the disease or condition is cancer, infection, autoimmune disease, or metabolic disease. In some embodiments, the immune checkpoint ligand is PD-L1. In some embodiments, the immune checkpoint ligand is a PD-L1 like ligand. In some embodiments, the antibody moiety is an scFv. In some embodiments, the antibody moiety is an scFv fused to an Fc fragment (such as a human IgG1 Fc).

In some embodiments, there is provided a method of treating an individual having a disease or condition, comprising: (a) administering to an individual an antibody agent comprising an anti-PD-L1 antibody moiety and a first conjugation moiety; (b) subsequently administering to the individual a label compound (e.g., a radionuclide compound) comprising a label (e.g., radionuclide) and a second conjugation moiety, wherein the first conjugation moiety and the second conjugation moiety are conjugated to each other in vivo to provide an imaging agent; (c) imaging the imaging agent in the individual with a non-invasive imaging technique; (d) diagnosing the individual as positive for PD-L1 if signal of the imaging agent is detected at a tissue of interest, or diagnosing the individual as negative for the immune checkpoint ligand if signal of the imaging agent is not detected at a tissue of interest; and (e) administering to the individual an effective amount of a therapeutic agent targeting PD-L1 or PD-1 (e.g., an inhibitor of PD-L1 or PD-1, such as an anti-PD-L1 antibody or anti-PD-1 antibody; or a radiolabeled molecule specifically binding PD-L1 or PD-1), if the individual is diagnosed as positive for PD-L1, wherein the anti-PD-L1 antibody moiety comprises: a V_{H} comprising a HC-CDR1 comprising the amino acid sequence of SEQ ID NO: 41, a HC-CDR2 comprising the amino acid sequence of SEQ ID NO: 42, and a HC-CDR3 comprising the amino acid sequence of SEQ ID NO: 43, or a variant thereof comprising up to about 5 amino acid substitutions; and a V_{L} comprising a LC-CDR1 comprising the amino acid sequence of SEQ ID NO: 44, a LC-CDR2 comprising the amino acid sequence of SEQ ID NO: 45, and a LC-CDR3 comprising the amino acid sequence of SEQ ID NO: 46, or a variant thereof comprising up to about 5 amino acid substitutions. In some embodiments, the method further comprises determining the expression level of PD-L1 in a tissue of interest in the individual based on signals emitted by the imaging agent from the tissue. In some embodiments, the first conjugation moiety and the second conjugation moiety are conjugated to each other via click chemistry. In some embodiments, the first conjugation moiety is a trans-cyclooctene (TCO) and the second conjugation moiety is a tetrazine (Tz), or the first conjugation moiety is a Tz and the second conjugation moiety is a TCO. In some embodiments, the radionuclide compound is administered immediately after the administration of the antibody agent. In some embodiments, the radionuclide compound is administered between about 1 hour and about 100 hours after the administration of the antibody agent. In some embodiments, the imaging is carried out between about 30 minutes and about 24 hours after administration of the radionuclide compound. In some embodiments, the antibody agent is administered intravenously, intraperitoneally, intramuscularly, subcutaneously, or orally. In some embodiments, the radionuclide compound is administered intravenously, intraperitoneally, intramuscularly, subcutaneously, or orally. In some embodiments, the method further comprises administering to the individual an antibody moiety not linked to a first conjugation moiety prior to the administration of the antibody agent. In some embodiments, the non-invasive imaging technique comprises single photon emission computed tomography (SPECT) imaging or positron emission tomography (PET) imaging. In some embodiments, the non-invasive imaging technique comprises or further comprises computed tomography imaging, magnetic resonance imaging, chemical luminescence imaging, or electrochemical luminescence imaging. In some embodiments, the method comprises imaging the individual over a period of time. In some embodiments, the anti-PD-L1 antibody moiety comprises: a V_{H} comprising the amino acid sequence of any one of SEQ ID NOs: 1, 5, 9, 11, and 13, or a variant thereof having at least about 80% (such as at least about any one of 80%, 85%, 90%, 95%, 96%, 97%, 98%, or 99%) sequence identity to the amino acid sequence of any one of SEQ ID NOs: 1, 5, 9, 11, and 13; and a V_{L} comprising the amino acid sequence of any one of SEQ ID NOs: 3, 7, 15, 17 and 19, or a variant thereof having at least about 80% (such as at least about any one of 80%, 85%, 90%, 95%, 96%, 97%, 98%, or 99%) sequence identity to the amino acid sequence of any one of SEQ ID NOs: 3, 7, 15, 17 and 19. In some embodiments, the anti-PD-L1 antibody moiety is humanized. In some embodiments, the radionuclide is selected from the group consisting of ⁶⁴Cu, ¹⁸F, ⁶⁷Ga, ⁶⁸Ga, ¹¹¹In, ¹⁷⁷Lu, ⁹⁰Y, ⁸⁹Zr, ⁶¹Cu, ⁶²Cu, ⁶⁷Cu, ¹⁹F, ⁶⁶Ga, ⁷²Ga, ⁴⁴Sc, ⁴⁷Sc, ⁸⁶Y, ⁸⁸Y and ⁴⁵Ti. In some embodiments, the radionuclide is ⁶⁸Ga. In some embodiments, the radionuclide compound comprises a chelating compound that chelates the radionuclide. In some embodiments, the chelating compound is NOTA, DOTA or derivatives thereof. In some embodiments, the disease or condition is cancer, infection, autoimmune disease, or metabolic disease. In some embodiments, the antibody moiety is an scFv. In some embodiments, the antibody moiety is an scFv fused to an Fc fragment (such as a human IgG1 Fc). In some embodiments, the scFv comprises one or more (such as 1, 2, 3, or more) engineered disulfide bonds. In some embodiments, the scFv comprises a first engineered cysteine residue at position 44 of V_{H} and a second engineered cysteine residue at position 100 of V_{L}, and/or a first engineered cysteine residue at position 105 of V_{H} and a second engineered cysteine residue at position 43 of V_{L}, wherein the first engineered cysteine residue and the second engineered cysteine residue form a disulfide bond, and wherein the amino acid positions are based on the Kabat numbering system. In some embodiments, the anti-PD-L1 antibody moiety comprises the amino acid sequence of any one of SEQ ID NOs: 25, 27, 29, 31, 33, 35, 37 and 39, or a variant thereof having at least about 80% (such as at least about any one of 80%, 85%, 90%, 95%, 96%, 97%, 98%, or 99%) sequence identity to the amino acid sequence of any one of SEQ ID NOs: 25, 27, 29, 31, 33, 35, 37 and 39.

In some embodiments, the individual has cancer. The cancer may comprise non-solid tumors (such as hematological tumors, for example, leukemias and lymphomas) or may comprise solid tumors. Exemplary cancers that may be diagnosed using the methods described herein, include, but are not limited to, carcinoma, blastoma, and sarcoma, and certain leukemia or lymphoid malignancies, benign and malignant tumors, and malignancies e.g., sarcomas, carcinomas, and melanomas. Adult tumors/cancers and pediatric tumors/cancers are also included. Solid or hematologic cancers discussed herein include, but is not limited to, Hodgkin lymphoma, non-Hodgkin lymphoma, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, Kaposi's sarcoma, soft tissue sarcoma, uterine sacronomasynovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, and melanoma.

The methods described herein are applicable to solid or hematologic cancers of all stages, including stages, I, II, III, and IV, according to the American Joint Committee on Cancer (AJCC) staging groups. In some embodiments, the solid or hematologic cancer is an/a: early stage cancer, non-metastatic cancer, primary cancer, advanced cancer, locally advanced cancer, metastatic cancer, or cancer in remission.

In some embodiments, the individual has a hematologic cancer. Exemplary hematologic cancers that can be diagnosed using the methods described herein include, but are not limited to, leukemia, lymphoma, acute lymphoblastic leukemia (ALL), acute non-lymphoblastic leukemia (ANLL), chronic lymphocytic leukemia (CLL), chronic myeloid leukemia (CML), non-Hodgkin lymphoma, and Hodgkin lymphoma.

In some embodiments, the individual has a solid tumor. Exemplary solid tumors that can be diagnosed using the methods described herein include, but are not limited to, colon tumor, melanoma, kidney tumor, ovarian tumor, lung tumor, breast tumor, and pancreatic tumor.

Cancer treatments can be evaluated, for example, by tumor regression, tumor weight or size shrinkage, time to progression, duration of survival, progression free survival, overall response rate, duration of response, quality of life, protein expression and/or activity. Approaches to determining efficacy of the therapy can be employed, including for example, measurement of response through radiological imaging.

In some embodiments, the individual has an infectious disease. The infection may be caused by a virus, bacteria, protozoa, or parasite. Exemplary pathogens include, but are not limited to, Acinetobacter baumannii, Anaplasma genus, Anaplasma phagocytophilum, Ancylostoma braziliense, Ancylostoma duodenale, Arcanobacterium haemolyticum, Ascaris lumbricoides, Aspergillus genus, Astroviridae, Babesia genus, Bacillus anthracis, Bacillus cereus, Bartonella henselae, BK virus, Blastocystis hominis, Blastomyces dermatitidis, Bordetella pertussis, Borrelia burgdorferi, Borrelia genus, Borrelia spp, Brucella genus, Brugia malayi, Bunyaviridae family, Burkholderia cepacia and other Burkholderia species, Burkholderia mallei, Burkholderia pseudomallei, Caliciviridae family, Campylobacter genus, Candida albicans, Candida spp, Chlamydia trachomatis, Chlamydophila pneumoniae, Chlamydophila psittaci, CJD prion, Clonorchis sinensis, Clostridium botulinum, Clostridium difficile, Clostridium perfringens, Clostridium perfringens, Clostridium spp, Clostridium tetani, Coccidioides spp, coronaviruses, Corynebacterium diphtheriae, Coxiella burnetii, Crimean-Congo hemorrhagic fever virus, Cryptococcus neoformans, Cryptosporidium genus, Cytomegalovirus (CMV), Dengue viruses (DEN-1, DEN-2, DEN-3 and DEN-4), Dientamoeba fragilis, Ebolavirus (EBOV), Echinococcus genus, Ehrlichia chaffeensis, Ehrlichia ewingii, Ehrlichia genus, Entamoeba histolytica, Enterococcus genus, Enterovirus genus, Enteroviruses, mainly Coxsackie A virus and Enterovirus 71 (EV71), Epidermophyton spp, Epstein-Barr Virus (EBV), Escherichia coli O157:H7, O111 and O104:H4, Fasciola hepatica and Fasciola gigantica, FFI prion, Filarioidea superfamily, Flaviviruses, Francisella tularensis, Fusobacterium genus, Geotrichum candidum, Giardia intestinalis, Gnathostoma spp, GSS prion, Guanarito virus, Haemophilus ducreyi, Haemophilus influenzae, Helicobacter pylori, Henipavirus (Hendra virus Nipah virus), Hepatitis A Virus, Hepatitis B Virus (HBV), Hepatitis C Virus (HCV), Hepatitis D Virus, Hepatitis E Virus, Herpes simplex virus 1 and 2 (HSV-1 and HSV-2), Histoplasma capsulatum, HIV (Human immunodeficiency virus), Hortaea werneckii, Human bocavirus (HBoV), Human herpesvirus 6 (HHFV-6) and Human herpesvirus 7 (HHV-7), Human metapneumovirus (hMPV), Human papillomavirus (HPV), Human parainfluenza viruses (HPIV), Human T cell leukemia virus 1 (HTLV-1), Japanese encephalitis virus, JC virus, Junin virus, Kaposi's Sarcoma associated herpesvirus (KSHV), Kingella kingae, Klebsiella granulomatis, Kuru prion, Lassa virus, Legionella pneumophila, Leishmania genus, Leptospira genus, Listeria monocytogenes, Lymphocytic choriomeningitis virus (LCMV), Machupo virus, Malassezia spp, Marburg virus, Measles virus, Metagonimus yokagawai, Microsporidia phylum, Molluscum contagiosum virus (MCV), Mumps virus, Mycobacterium leprae and Mycobacterium lepromatosis, Mycobacterium tuberculosis, Mycobacterium ulcerans, Mycoplasma pneumoniae, Naegleria fowleri, Necator americanus, Neisseria gonorrhoeae, Neisseria meningitidis, Nocardia asteroides, Nocardia spp, Onchocerca volvulus, Orientia tsutsugamushi, Orthomyxoviridae family (Influenza), Paracoccidioides brasiliensis, Paragonimus spp, Paragonimus westermani, Parvovirus B19, Pasteurella genus, Plasmodium genus, Pneumocystis jirovecii, Poliovirus, Rabies virus, Respiratory syncytial virus (RSV), Rhinovirus, rhinoviruses, Rickettsia akari, Rickettsia genus, Rickettsia prowazekii, Rickettsia rickettsii, Rickettsia typhi, Rift Valley fever virus, Rotavirus, Rubella virus, Sabia virus, Salmonella genus, Sarcoptes scabiei, SARS coronavirus, Schistosoma genus, Shigella genus, Sin Nombre virus, Hantavirus, Sporothrix schenckii, Staphylococcus genus, Staphylococcus genus, Streptococcus agalactiae, Streptococcus pneumoniae, Streptococcus pyogenes, Strongyloides stercoralis, Taenia genus, Taenia solium, Tick-borne encephalitis virus (TBEV), Toxocara canis or Toxocara cati, Toxoplasma gondii, Treponema pallidum, Trichinella spiralis, Trichomonas vaginalis, Trichophyton spp, Trichuris trichiura, Trypanosoma brucei, Trypanosoma cruzi, Ureaplasma urealyticum, Varicella zoster virus (VZV), Varicella zoster virus (VZV), Variola major or Variola minor, vCJD prion, Venezuelan equine encephalitis virus, Vibrio cholerae, West Nile virus, Western equine encephalitis virus, Wuchereria bancrofti, Yellow fever virus, Yersinia enterocolitica, Yersinia pestis, and Yersinia pseudotuberculosis.

In some embodiments, the individual has an autoimmune disease. Exemplary autoimmune disease include, but are not limited to, Behcet disease, systemic lupus erythematosus, multiple sclerosis (systemic scleroderma and progressive systemic scleroderma), scleroderma, polymyositis, dermatomyositis, periarteritis nodosa (polyarteritis nodosa and microscopic polyangiitis), aortitis syndrome (Takayasu arteritis), malignant rheumatoid arthritis, rheumatoid arthritis, Wegner's granulomatosis, mixed connective tissue disease, Sjogren syndrome, adult-onset Still's disease, allergic granulomatous angiitis, hypersensitivity angiitis, Cogan's syndrome, RS3PE, temporal arteritis, polymyalgia rheumatica, fibromyalgia syndrome, antiphospholipid antibody syndrome, eosinophilic fasciitis, IgG4-related diseases (e.g., primary sclerosing cholangitis and autoimmune pancreatitis), Guillain-Barre syndrome, myasthenia gravis, chronic atrophic gastritis, autoimmune hepatitis, primary biliary cirrhosis, aortitis syndrome, Goodpasture's syndrome, rapidly progressive glomerulonephritis, megaloblastic anemia, autoimmune hemolytic anemia, autoimmune neutropenia, idiopathic thrombocytopenic purpura, Graves' disease (hyperthyroidism), Hashimoto's thyroiditis, autoimmune adrenal insufficiency, primary hypothyroidism, idiopathic Addison's disease (chronic adrenal insufficiency), type I diabetes mellitus, chronic discoid lupus erythematosus, localized scleroderma, psoriasis, psoriatic arthritis, pemphigus, pemphigoid, herpes gestationis, linear IgA bullous skin disease, epidermolysis bullosa acquisita, alopecia areata, vitiligo, Harada disease, autoimmune optic neuropathy, idiopathic azoospermia, recurrent fetal loss, and inflammatory bowel diseases (ulcerative colitis and Crohn's disease).

In some embodiments, the individual has a metabolic disease associated with abnormal immune response. Exemplary metabolic diseases include, but are not limited to, inflammatory bowel disease, multiple sclerosis, psoriasis, rheumatoid arthritis, and systemic lupus erythematosus.

### III. Antibody Agents

Also provided herein are antibody agents comprising antibody moieties that specifically recognize an immune checkpoint ligand, such as PD-L1 and PD-L1 like ligands. Such antibody agents and antibody moieties derived therefrom can be incorporated into the methods and antibody agents described in the sections above. Suitable antibody moieties include, but are not limited to, scFv, Fab, scFv fused to an Fc fragment (also referred herein as “scFv-Fc”), scFv fused to another Fv fragment (also referred herein as “scFv-Fv”). The antibody moieties (including the anti-PD-L1 antibody agents) described herein may have any one or more of the features described in the sections a)-h) below.

In some embodiments, the antibody moiety comprises an scFv. In some embodiments, the antibody moiety is an scFv. In some embodiments, the scFv has the configuration of (from N-terminus to C-terminus): V_{L}-L-V_{H}, or V_{H}-L-V_{L}, wherein L is a peptide linker. In some embodiments, the scFv is chimeric, human, partially humanized, fully humanized, or semi-synthetic.

In some embodiments, the scFv is engineered to have enhanced thermal stability. In some embodiments, the scFv is engineered to have a melting temperature of about 55-70° C., such as about any one of 55-60, 60-65, or 65-70° C. In some embodiments, the scFv comprises one or more (such as 1, 2, 3, or more) engineered disulfide bonds. In some embodiments, the scFv comprises a first engineered cysteine residue at position 44 of V_{H} and a second engineered cysteine residue at position 100 of V_{L}, and/or a first engineered cysteine residue at position 105 of V_{H} and a second engineered cysteine residue at position 43 of V_{L}, wherein the first engineered cysteine residue and the second engineered cysteine residue form a disulfide bond, and wherein the amino acid positions are based on the Kabat numbering system. Other engineered disulfide bonds may be introduced into the scFv by engineering a cysteine in the VH and a cysteine in the VL at suitable positions based on the structure and sequences of the scFv.

In some embodiments, the antibody moiety comprises an Fc fragment. In some embodiments, the antibody moiety is an scFv fused to an Fc fragment. In some embodiments, the antibody moiety comprises a scFv fused to an Fc fragment via a peptide linker. In some embodiments, the Fc fragment is a human IgG1 Fc fragment. In some embodiments, the Fc fragment comprises one or more mutations to increase clearance or decrease half-life. For example, the Fc fragment may have H310A and/or H435Q mutations, wherein the amino acid positions are based on the Kabat numbering system.

In some embodiments, the Fc fragment comprises an immunoglobulin IgG heavy chain constant region comprising a hinge region (starting at Cys226), an IgG CH2 domain and CH3 domain. The term “hinge region” or “hinge sequence” as used herein refers to the amino acid sequence located between the linker and the CH2 domain. In some embodiments, the fusion protein comprises an Fc fragment comprising a hinge region. In some embodiments, the Fc fragment of the fusion protein starts at the hinge region and extends to the C-terminus of the IgG heavy chain. In some embodiments, the fusion protein comprises an Fc fragment that does not comprise the hinge region.

In some embodiments, the antibody moiety comprises an Fc fragment selected from the group consisting of Fc fragments from IgG, IgA, IgD, IgE, IgM, and combinations and hybrids thereof. In some embodiments, the Fc fragment is derived from a human IgG. In some embodiments, the Fc fragment comprises the Fc region of human IgG1, IgG2, IgG3, IgG4, or a combination or hybrid IgG. In some embodiments, the Fc fragment is an IgG1 Fc fragment. In some embodiments, the Fc fragment comprises the CH2 and CH3 domains of IgG1. In some embodiments, the Fc fragment is an IgG4 Fc fragment. In some embodiments, the Fc fragment comprises the CH2 and CH3 domains of IgG4. IgG4 Fc is known to exhibit less effector activity than IgG1 Fc, and thus may be desirable for some applications. In some embodiments, the Fc fragment is derived from of a mouse immunoglobulin.

In some embodiments, the IgG CH2 domain starts at Ala231. In some embodiments, the CH3 domain starts at Gly341. It is understood that the C-terminus Lys residue of human IgG can be optionally absent. It is also understood that conservative amino acid substitutions of the Fc region without affecting the desired structure and/or stability of Fc is contemplated within the scope of the invention.

In some embodiments, each chain of the Fc fragment is fused to the same antibody moiety. In some embodiments, the scFv-Fc comprises two identical scFvs described herein, each fused with one chain of the Fc fragment. In some embodiments, the scFv-Fc is a homodimer.

In some embodiments, the scFv-Fc comprises two different scFvs, each fused with one chain of the Fc fragment. In some embodiments, the scFv-Fc is a heterodimer. Heterodimerization of non-identical polypeptides in the scFv-Fc can be facilitated by methods known in the art, including without limitation, heterodimerization by the knob-into-hole technology. The structure and assembly method of the knob-into-hole technology can be found in, e.g., U.S. Pat. Nos. 5,821,333, 7,642,228, US 2011/0287009 and PCT/US2012/059810, hereby incorporated by reference in their entireties. This technology was developed by introducing a “knob” (or a protuberance) by replacing a small amino acid residue with a large one in the CH3 domain of one Fc and introducing a “hole” (or a cavity) in the CH3 domain of the other Fc by replacing one or more large amino acid residues with smaller ones. In some embodiments, one chain of the Fc fragment in the fusion protein comprises a knob, and the second chain of the Fc fragment comprises a hole.

The preferred residues for the formation of a knob are generally naturally occurring amino acid residues and are preferably selected from arginine (R), phenylalanine (F), tyrosine (Y) and tryptophan (W). Most preferred are tryptophan and tyrosine. In one embodiment, the original residue for the formation of the knob has a small side chain volume, such as alanine, asparagine, aspartic acid, glycine, serine, threonine or valine. Exemplary amino acid substitutions in the CH3 domain for forming the knob include without limitation the T366W, T366Y or F405W substitution.

The preferred residues for the formation of a hole are usually naturally occurring amino acid residues and are preferably selected from alanine (A), serine (S), threonine (T) and valine (V). In one embodiment, the original residue for the formation of the hole has a large side chain volume, such as tyrosine, arginine, phenylalanine or tryptophan. Exemplary amino acid substitutions in the CH3 domain for generating the hole include without limitation the T366S, L368A, F405A, Y407A, Y407T and Y407V substitutions. In certain embodiments, the knob comprises T366W substitution, and the hole comprises the T366S/L368A/Y 407V substitutions. It is understood that other modifications to the Fc region known in the art that facilitate heterodimerization are also contemplated and encompassed by the instant application.

Other scFv-Fc variants (including variants of isolated anti-PD-L1 scFv-Fc, e.g., a full-length anti-PD-L1 antibody variants) comprising any of the variants described herein (e.g., Fc variants, effector function variants, glycosylation variants, cysteine engineered variants), or combinations thereof, are contemplated.

### a) Antibody Affinity

Binding specificity of the antibody moieties can be determined experimentally by methods known in the art. Such methods comprise, but are not limited to Western blots, ELISA-, RIA-, ECL-, IRMA-, EIA-, BIACORE™-tests and peptide scans.

In some embodiments, the K_{D} of the binding between the antibody moiety and the immune checkpoint ligand (e.g., PD-L1 or a PD-L1 like ligand) is about 10⁻⁷ M to about 10⁻¹² M, about 10⁻⁷ M to about 10⁻⁸ M, about 10⁻⁸ M to about 10⁻⁹ M, about 10⁻⁹ M to about 10⁻¹⁰ M, about 10⁻¹⁰ M to about 10⁻¹¹ M, about 10⁻¹¹ M to about 10⁻¹² M, about 10⁻⁷ M to about 10⁻¹² M, about 10⁻⁸ M to about 10⁻¹² M, about 10⁻⁹ M to about 10⁻¹² M, about 10⁻¹⁰ M to about 10⁻¹² M, about 10⁻⁷ M to about 10⁻¹¹ M, about 10⁻⁸ M to about 10⁻¹¹ M, about 10⁻⁹ M to about 10⁻¹¹ M, about 10⁻⁷ M to about 10⁻¹⁰ M, about 10⁻⁸ M to about 10⁻¹⁰ M, or about 10⁻⁷ M to about 10⁻⁹ M. In some embodiments, the K_{D} of the binding between the antibody moiety and the immune checkpoint ligand (e.g., PD-L1 or a PD-L1 like ligand) is stronger than about any one of 10⁻⁷ M, 10⁻⁸ M, 10⁻⁹ M, 10⁻¹⁰ M, 10⁻¹¹ M, or 10⁻¹² M. In some embodiments, the immune checkpoint ligand is human immune checkpoint ligand (e.g., human PD-L1 or a PD-L1 like ligand). In some embodiments, the immune checkpoint ligand is cynomolgus monkey immune checkpoint ligand (e.g., cynomolgus monkey PD-L1 or a PD-L1 like ligand). In some embodiments, the antibody moiety specifically recognizes an epitope in the extracellular domain of the immune checkpoint ligand, such as amino acids 19-238 of SEQ ID NO: 4.

In some embodiments, the Kₒₙ of the binding between the antibody moiety and the immune checkpoint ligand (e.g., PD-L1 or a PD-L1 like ligand) is about 10³ M⁻¹ s⁻¹ to about 10⁸ M⁻¹ s⁻¹, about 10³ M⁻¹ s⁻¹ to about 10⁴ M⁻¹ s⁻¹, about 10⁴ M⁻¹ s⁻¹ to about 10⁵ M⁻¹ s⁻¹, about 10⁵ M⁻¹ s⁻¹ to about 10⁶ M⁻¹ s⁻¹, about 10⁶ M⁻¹ s⁻¹ to about 10⁷ M⁻¹ s⁻¹, or about 10⁷ M⁻¹ s⁻¹ to about 10⁸ M⁻¹ s⁻¹. In some embodiments, the Kₒₙ of the binding between the antibody moiety and the immune checkpoint ligand (e.g., PD-L1 or a PD-L1 like ligand) is about 10³ M s⁻¹ to about 10⁵ M⁻¹ s⁻¹, about 10⁴ M⁻¹ s⁻¹ to about 10⁶ M⁻¹ s⁻¹, about 10⁵ M⁻¹ s⁻¹ to about 10⁷ M⁻¹ s⁻¹, about 10⁶ M⁻¹ s⁻¹ to about 10⁸ M⁻¹ s⁻¹, about 10⁴ M⁻¹ s⁻¹ to about 10⁷ M⁻¹ s⁻¹, or about 10⁵ M⁻¹ s⁻¹ to about 10⁸ M⁻¹ s⁻¹. In some embodiments, the Kₒₙ of the binding between the antibody moiety and the immune checkpoint ligand (e.g., PD-L1 or a PD-L1 like ligand) is no more than about any one of 10³ M⁻¹ s⁻¹, 10⁴ M⁻¹ s⁻¹, 10⁵ M⁻¹ s⁻¹, 10⁶ M⁻¹ s⁻¹, 10⁷ M⁻¹ s⁻¹ or 10⁸ M⁻¹ s⁻¹.

In some embodiments, the K_{off} of the binding between the antibody moiety and the immune checkpoint ligand (e.g., PD-L1 or a PD-L1 like ligand) is about 1 s⁻¹ to about 10⁻⁶ s⁻¹, about 1 s to about 10⁻² s⁻¹, about 10⁻² s⁻¹ to about 10⁻³ s⁻¹, about 10⁻³ s⁻¹ to about 10⁻⁴ s⁻¹, about 10⁻⁴ s⁻¹ to about 10⁻⁵ s⁻¹, about 10⁻⁵ s⁻¹ to about 10⁻⁶ s⁻¹, about 1 s⁻¹ to about 10⁻⁵ s⁻¹, about 10⁻² s⁻¹ to about 10⁻⁶ s⁻¹, about 10⁻³ s⁻¹ to about 10⁻⁶ s⁻¹, about 10⁻⁴ s⁻¹ to about 10⁻⁶ s⁻¹, about 10⁻² s⁻¹ to about 10⁻⁵ s⁻¹, or about 10⁻³ s⁻¹ to about 10⁻⁵ s⁻¹. In some embodiments, the K_{off} of the binding between the antibody moiety and the immune checkpoint ligand (e.g., PD-L1 or a PD-L1 like ligand) is at least about any one of 1 s⁻¹, 10⁻² s⁻¹, 10⁻³ s⁻¹, 10⁻⁴ s⁻¹, 10⁻⁵ s⁻¹ or 10⁻⁶ s⁻¹.

### b) Chimeric or Humanized Antibodies

In some embodiments, the antibody moiety is a chimeric antibody. Certain chimeric antibodies are described, e.g., in U.S. Pat. No. 4,816,567; and Morrison et al., Proc. Natl. Acad. Sci. USA, 81:6851-6855 (1984)). In some embodiments, a chimeric antibody comprises a non-human variable region (e.g., a variable region derived from mouse) and a human constant region. In some embodiments, a chimeric antibody is a “class switched” antibody in which the class or subclass has been changed from that of the parent antibody. Chimeric antibodies include antigen-binding fragments thereof.

In some embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing SDR (a-CDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); Framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

### c) Human Antibodies

In some embodiments, the antibody moiety is a human antibody (known as human domain antibody, or human DAb). Human antibodies can be produced using various techniques known in the art. Human antibodies are described generally in van Dijk and van de Winkel, Curr. Opin. Pharmacol. 5: 368-74 (2001), Lonberg, Curr. Opin. Immunol. 20:450-459 (2008), and Chen, Mol. Immunol. 47(4):912-21 (2010). Transgenic mice or rats capable of producing fully human single-domain antibodies (or DAb) are known in the art. See, e.g., US20090307787A1, U.S. Pat. No. 8,754,287, US20150289489A1, US20100122358A1, and WO2004049794.

Human antibodies (e.g., human DAbs) may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies (e.g., human DAbs) can also be made by hybridoma-based methods. Human myeloma and mouse-human heteromyeloma cell lines for the production of human monoclonal antibodies have been described (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991)). Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies (e.g., human DAbs) may also be generated by isolating Fv clone variable domain sequences selected from human-derived phage display libraries. Such variable domain sequences may then be combined with a desired human constant domain. Techniques for selecting human antibodies from antibody libraries are described below.

### d) Library-Derived Antibodies

The antibody moieties may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, N.J., 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004). Methods for constructing single-domain antibody libraries have been described, for example, see U.S. Pat. No. 7,371,849.

In certain phage display methods, repertoires of V_{H} and V_{L} genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically displays antibody fragments, either as scFv fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self-antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

### e) Substitution, Insertion, Deletion and Variants

In some embodiments, antibody variants having one or more amino acid substitutions are provided. Sites of interest for substitutional mutagenesis include the HVRs (or CDRs) and FRs. Conservative substitutions are shown in Table 2 under the heading of “Preferred substitutions.” More substantial changes are provided in Table 2 under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE 2
Amino acid substitutions

| Original | | Preferred |
|---|---|---|
| Residue | Exemplary Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; and (6) aromatic: Trp, Tyr, Phe.

Non-conservative substitutions will entail exchanging a member of one of these classes for another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g. binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or SDRs (a-CDRs), with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, N.J., (2001)). In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In some embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may be outside of HVR “hotspots” or CDRs. In some embodiments of the variant V_{H}H sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

### f) Glycosylation Variants

In some embodiments, the antibody moiety is altered to increase or decrease the extent to which the construct is glycosylated. Addition or deletion of glycosylation sites to an antibody may be conveniently accomplished by altering the amino acid sequence such that one or more glycosylation sites is created or removed.

Where the antibody moiety comprises an Fc region (e.g., scFv-Fc), the carbohydrate attached thereto may be altered. Native antibodies produced by mammalian cells typically comprise a branched, biantennary oligosaccharide that is generally attached by an N-linkage to Asn297 of the C_{H}2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in the antibody moiety may be made in order to create antibody variants with certain improved properties.

In some embodiments, the antibody moiety has a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g., complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Patent Application No. US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

In some embodiments, the antibody moiety has bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

### g) Fc Region Variants

In some embodiments, one or more amino acid modifications may be introduced into the Fc region of the antibody moiety (e.g., scFv-Fc), thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In some embodiments, the Fc fragment possesses some but not all effector functions, which make it a desirable candidate for applications in which the half-life of the antibody moiety in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 2 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); U.S. Pat. No. 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, Calif.; and CytoTox 96® non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half-life determinations can also be performed using methods known in the art (see, e.g., Petkova, S. B. et al., Int'l. Immunol. 18(12):1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In some embodiments, the antibody moiety comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

In some embodiments, the antibody moiety (e.g., scFv-Fc) variant comprising a variant Fc region comprising one or more amino acid substitutions which alters half-life and/or changes binding to the neonatal Fc receptor (FcRn). Antibodies with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which alters binding of the Fc region to FcRn. Such Fc variants include those with substitutions at one or more of Fc region residues, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc region variants.

### h) Cysteine Engineered Antibody Variants

In some embodiments, it may be desirable to create cysteine engineered antibody moieties, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In some embodiments, any one or more of the following residues may be substituted with cysteine: A118 (EU numbering) of the heavy chain; and S400 (EU numbering) of the heavy chain Fc region. Cysteine engineered antibody moieties may be generated as described, e.g., in U.S. Pat. No. 7,521,541.

### i) Antibody Derivatives

In some embodiments, the antibody moiety described herein may be further modified to comprise additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, proylpropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in diagnosis under defined conditions, etc.

In some embodiments, the antibody moiety may be further modified to comprise one or more biologically active protein, polypeptides or fragments thereof. “Bioactive” or “biologically active”, as used herein interchangeably, means showing biological activity in the body to carry out a specific function. For example, it may mean the combination with a particular biomolecule such as protein, DNA, etc., and then promotion or inhibition of the activity of such biomolecule. In some embodiments, the bioactive protein or fragments thereof include proteins and polypeptides that are administered to patients as the active drug substance for prevention of or treatment of a disease or condition, as well as proteins and polypeptides that are used for diagnostic purposes, such as enzymes used in diagnostic tests or in vitro assays, as well as proteins and polypeptides that are administered to a patient to prevent a disease such as a vaccine.

### IV. Anti-PD-L1 Antibody Agents

One aspect of the present application provides an anti-PD-L1 antibody agent that comprises an anti-PD-L1 antibody moiety or any of the variants thereof as described in Section III.

In some embodiments, there is provided an anti-PD-L1 antibody agent comprising any one of the anti-PD-L1 antibody moieties described herein. In some embodiments, the anti-PD-L1 antibody is a full length antibody (e.g., a full length antibody not attached to a conjugation moiety). In some embodiments, the anti-PD-L1 antibody agent is an antibody fragment (e.g., an antibody fragment not attached to a conjugation moiety). In some embodiments, the anti-PD-L1 antibody moiety is humanized. In some embodiments, the anti-PD-L1 antibody moiety comprises an scFv. In some embodiments, the anti-PD-L1 antibody moiety is an scFv. In some embodiments, the scFv comprises a first engineered cysteine residue at position 44 of V_{H} and a second engineered cysteine residue at position 100 of V_{L}, or a first engineered cysteine residue at position 105 of V_{H} and a second engineered cysteine residue at position 43 of V_{L}, wherein the first engineered cysteine residue and the second engineered cysteine residue form a disulfide bond, and wherein the amino acid positions are based on the Kabat numbering system. In some embodiments, the anti-PD-L1 antibody moiety is an scFv fused to an Fc fragment (such as IgG1 Fc fragment). In some embodiments, the Fc fragment has H310A and H435Q mutations, wherein the amino acid positions are based on the Kabat numbering system.

In some embodiments, there is provided an antibody agent comprising an antibody moiety, wherein the antibody moiety is an antibody or an antigen-binding fragment thereof, wherein the antibody or antigen-binding fragment thereof comprises a heavy chain variable region (V_{H}) and a light chain variable region (V_{L}), wherein: the V_{H} comprises a heavy chain complementarity determining region 1 (HC-CDR1) comprising the amino acid sequence of SEQ ID NO: 41, an HC-CDR2 comprising the amino acid sequence of SEQ ID NO: 42, and an HC-CDR3 comprising the amino acid sequence of SEQ ID NO: 43, or a variant thereof comprising up to a total of about 5 amino acid substitutions in the HC-CDRs; and the V_{L} comprises a light chain complementarity determining region 1 (LC-CDR1) comprising the amino acid sequence of SEQ ID NO: 44, an LC-CDR2 comprising the amino acid sequence of SEQ ID NO: 45, and an LC-CDR3 comprising the amino acid sequence of SEQ ID NO: 46, or a variant thereof comprising up to a total of about 5 amino acid substitutions in the LC-CDRs. In some embodiments, the anti-PD-L1 antibody moiety comprises: a V_{H} comprising the amino acid sequence of any one of SEQ ID NOs: 1, 5, 9, 11, and 13, or a variant thereof having at least about 80% (such as at least about any one of 80%, 85%, 90%, 95%, 96%, 97%, 98%, or 99%) sequence identity to the amino acid sequence of any one of SEQ ID NOs: 1, 5, 9, 11, and 13; and a V_{L} comprising the amino acid sequence of any one of SEQ ID NOs: 3, 7, 15, 17 and 19, or a variant thereof having at least about 80% (such as at least about any one of 80%, 85%, 90%, 95%, 96%, 97%, 98%, or 99%) sequence identity to the amino acid sequence of any one of SEQ ID NOs: 3, 7, 15, 17 and 19. In some embodiments, the anti-PD-L1 antibody moiety is humanized.

In some embodiments, there is provided an anti-PD-L1 antibody agent comprising an anti-PD-L1 scFv comprising: a V_{H} comprising a heavy chain complementarity determining region 1 (HC-CDR1) comprising the amino acid sequence of SEQ ID NO: 41, an HC-CDR2 comprising the amino acid sequence of SEQ ID NO: 42, and an HC-CDR3 comprising the amino acid sequence of SEQ ID NO: 43, or a variant thereof comprising up to a total of about 5 amino acid substitutions in the HC-CDRs; and a V_{L} comprising a light chain complementarity determining region 1 (LC-CDR1) comprising the amino acid sequence of SEQ ID NO: 44, an LC-CDR2 comprising the amino acid sequence of SEQ ID NO: 45, and an LC-CDR3 comprising the amino acid sequence of SEQ ID NO: 46, or a variant thereof comprising up to a total of about 5 amino acid substitutions in the LC-CDRs. In some embodiments, the anti-PD-L1 antibody moiety comprises: a V_{H} comprising the amino acid sequence of any one of SEQ ID NOs: 1, 5, 9, 11, and 13, or a variant thereof having at least about 80% (such as at least about any one of 80%, 85%, 90%, 95%, 96%, 97%, 98%, or 99%) sequence identity to the amino acid sequence of any one of SEQ ID NOs: 1, 5, 9, 11, and 13; and a V_{L} comprising the amino acid sequence of any one of SEQ ID NOs: 3, 7, 15, 17 and 19, or a variant thereof having at least about 80% (such as at least about any one of 80%, 85%, 90%, 95%, 96%, 97%, 98%, or 99%) sequence identity to the amino acid sequence of any one of SEQ ID NOs: 3, 7, 15, 17 and 19. In some embodiments, the anti-PD-L1 antibody moiety is humanized. In some embodiments, the anti-PD-L1 scFv comprises a first engineered cysteine residue at position 44 of V_{H} and a second engineered cysteine residue at position 100 of V_{L}, or a first engineered cysteine residue at position 105 of V_{H} and a second engineered cysteine residue at position 43 of V_{L}, wherein the first engineered cysteine residue and the second engineered cysteine residue form a disulfide bond, and wherein the amino acid positions are based on the Kabat numbering system. In some embodiments, the anti-PD-L1 scFv comprises the amino acid sequence of any one of SEQ ID NOs: 25, 27, 29, 31, 33, 35, 37 and 39, or a variant thereof having at least about 80% (such as at least about any one of 80%, 85%, 90%, 95%, 96%, 97%, 98%, or 99%) sequence identity to the amino acid sequence of any one of SEQ ID NOs: 25, 27, 29, 31, 33, 35, 37 and 39.

In some embodiments, there is provided an anti-PD-L1 antibody agent comprising an antibody moiety, wherein said antibody moiety is an antibody or an antigen-binding fragment thereof, comprising: (a) a VH CDR1, a VH CDR2, and a VH CDR3, respectively comprising the amino acid sequences of a CDR1, a CDR2, and a CDR3 within a VH chain region having the sequence set forth in any of SEQ ID NOs: 1, 5, 9, 11, and 13, or a variant thereof having at least about 80% (such as at least about any one of 80%, 85%, 90%, 95%, 96%, 97%, 98%, or 99%) sequence identity to the amino acid sequence of any one of SEQ ID NOs: 1, 5, 9, 11, and 13; and a VL CDR1, a VL CDR2, and a VL CDR3, respectively comprising the amino acid sequences of a CDR1, a CDR2, and a CDR3 within a VL chain region having the sequence set forth in any of SEQ ID NOs: 3, 7, 15, 17 and 19, or a variant thereof having at least about 80% (such as at least about any one of 80%, 85%, 90%, 95%, 96%, 97%, 98%, or 99%) sequence identity to the amino acid sequence of any one of SEQ ID NOs: 3, 7, 15, 17 and 19.

In some embodiments, there is provided an anti-PD-L1 antibody agent (such as an isolated anti-PD-L1 antibody agent) comprising an antibody moiety, wherein said antibody moiety is an antibody or an antigen-binding fragment thereof, wherein said antibody or antigen-binding fragment thereof specifically binds to PD-L1 in competition with an anti-PD-L1 antibody or an antigen-binding fragment thereof, wherein said anti-PD-L1 antibody or antigen-binding fragment thereof comprises a VH and a VL, wherein: the VH comprises a heavy chain complementarity determining region 1 (HC-CDR1) comprising the amino acid sequence of SEQ ID NO: 41, an HC-CDR2 comprising the amino acid sequence of SEQ ID NO: 42, and an HC-CDR3 comprising the amino acid sequence of SEQ ID NO: 43, or a variant thereof comprising up to a total of about 5 amino acid substitutions in the HC-CDRs; and the VL comprises a light chain complementarity determining region 1 (LC-CDR1) comprising the amino acid sequence of SEQ ID NO: 44, an LC-CDR2 comprising the amino acid sequence of SEQ ID NO: 45, and an LC-CDR3 comprising the amino acid sequence of SEQ ID NO: 46, or a variant thereof comprising up to a total of about 5 amino acid substitutions in the LC-CDRs.

In some embodiments, there is provided an anti-PD-L1 antibody agent (such as an isolated anti-PD-L1 antibody agent) comprising an anti-PD-L1 scFv fused to an Fc fragment, wherein the anti-PD-L1 scFv comprises: a V_{H} comprising a heavy chain complementarity determining region 1 (HC-CDR1) comprising the amino acid sequence of SEQ ID NO: 41, an HC-CDR2 comprising the amino acid sequence of SEQ ID NO: 42, and an HC-CDR3 comprising the amino acid sequence of SEQ ID NO: 43, or a variant thereof comprising up to a total of about 5 amino acid substitutions in the HC-CDRs; and a V_{L} comprising a light chain complementarity determining region 1 (LC-CDR1) comprising the amino acid sequence of SEQ ID NO: 44, an LC-CDR2 comprising the amino acid sequence of SEQ ID NO: 45, and an LC-CDR3 comprising the amino acid sequence of SEQ ID NO: 46, or a variant thereof comprising up to a total of about 5 amino acid substitutions in the LC-CDRs. In some embodiments, the anti-PD-L1 antibody moiety comprises: a V_{H} comprising the amino acid sequence of any one of SEQ ID NOs: 1, 5, 9, 11, and 13, or a variant thereof having at least about 80% (such as at least about any one of 80%, 85%, 90%, 95%, 96%, 97%, 98%, or 99%) sequence identity to the amino acid sequence of any one of SEQ ID NOs: 1, 5, 9, 11, and 13; and a V_{L} comprising the amino acid sequence of any one of SEQ ID NOs: 3, 7, 15, 17 and 19, or a variant thereof having at least about 80% (such as at least about any one of 80%, 85%, 90%, 95%, 96%, 97%, 98%, or 99%) sequence identity to the amino acid sequence of any one of SEQ ID NOs: 3, 7, 15, 17 and 19. In some embodiments, the anti-PD-L1 antibody moiety is humanized. In some embodiments, the anti-PD-L1 scFv comprises a first engineered cysteine residue at position 44 of V_{H} and a second engineered cysteine residue at position 100 of V_{L}, or a first engineered cysteine residue at position 105 of V_{H} and a second engineered cysteine residue at position 43 of V_{L}, wherein the first engineered cysteine residue and the second engineered cysteine residue form a disulfide bond, and wherein the amino acid positions are based on the Kabat numbering system. In some embodiments, the Fc fragment is an IgG1 Fc fragment. In some embodiments, the Fc fragment has H310A and H435Q mutations, wherein the amino acid positions are based on the Kabat numbering system. In some embodiments, the anti-PD-L1 scFv comprises the amino acid sequence of any one of SEQ ID NOs: 25, 27, 29, 31, 33, 35, 37 and 39, or a variant thereof having at least about 80% (such as at least about any one of 80%, 85%, 90%, 95%, 96%, 97%, 98%, or 99%) sequence identity to the amino acid sequence of any one of SEQ ID NOs: 25, 27, 29, 31, 33, 35, 37 and 39. In some embodiments, the antibody moiety comprises an amino acid sequence having at least about 80% sequence identity (such as at least about any one of 80%, 85%, 90%, 95%, 96%, 97%, 98%, or 99%) to the amino acid sequence of SEQ ID NO: 21 or 23.

In some embodiments, there is provided an antibody agent comprising an antibody moiety, wherein said antibody moiety is an antibody or an antigen-binding fragment thereof, wherein said antibody or antigen-binding fragment thereof specifically binds to PD-L1 competitively with an anti-PD-L1 antibody comprising a VH and a VL, wherein: a) the VH comprises a heavy chain complementarity determining region 1 (HC-CDR1) comprising the amino acid sequence of SEQ ID NO: 41, an HC-CDR2 comprising the amino acid sequence of SEQ ID NO: 42, and an HC-CDR3 comprising the amino acid sequence of SEQ ID NO: 43; and b) the V_{L} comprises a light chain complementarity determining region 1 (LC-CDR1) comprising the amino acid sequence of SEQ ID NO: 44, an LC-CDR2 comprising the amino acid sequence of SEQ ID NO: 45, and an LC-CDR3 comprising the amino acid sequence of SEQ ID NO: 46.

In some embodiments, the half-life of the antibody agent is between about 10 minutes and about 8 days in serum. In some embodiments, the binding between the antibody moiety and the immune checkpoint ligand has a K_{D} between about 9×10⁻¹⁰ M to about 1×10⁻⁸ M with PD-L1. In some embodiments, the molecular weight of the antibody moiety is no more than about 400 kDa (such as no more than about 350 kDa, 300 kDa, 250 kDa, 200 kDa, 150 kDa). In some embodiments, the antibody moiety cross-reacts with PD-L1 from a non-human mammal. In some embodiments, the antibody moiety is chimeric, humanized, or fully human. In some embodiments, the antibody moiety is stable at acidic pH. In some embodiments, the antibody moiety has a melting temperature (Tm) of about 55-70° C. In some embodiments, the antibody moiety is selected from the group consisting of a single-chain Fv (scFv), a Fab, a Fab′, a F(ab′)2, an Fv fragment, a disulfide stabilized Fv fragment (dsFv), a (dsFv)2, a VHH, a Fv-Fc fusion, a scFv-Fc fusion, a scFv-Fv fusion, a diabody, a tribody, and a tetrabody. A diabody, tribody or a tetrabody described herein are comtemplated to include any of the antibody moiety described herein. In some embodiments, the antibody moiety has an isotype selected from the group consisting of an IgG, an IgM, an IgA, an IgD, and an IgE. In some embodiments, the antibody moiety comprises an scFv fused to an Fc fragment. In some embodiments, the Fc fragment is selected from the group consisting of an IgG1 Fc fragment, an IgG2 Fc fragment, an IgG3 Fc fragment, an IgG4 Fc fragment, an IgA Fc fragment, an IgM Fc fragment, an IgE Fc fragment, and an IgD Fc fragment. In some embodiments, the Fc fragment is an IgG1 Fc fragment. In some embodiments, the Fc fragment is a human IgG1 Fc fragment. In some embodiments, the Fc fragment comprises H310A and H435Q mutations, wherein the amino acid positions are based on the Kabat numbering system.

In some embodiments, the anti-PD-L1 antibody agent described herein further comprises any conjugation moiety described herein. In some embodiments, the conjugation moiety comprises a member of a click chemistry pair. In some embodiments, the chemistry pair is selected from the group consisting of a TCO-Tz pair, an azide-alkyne pair, an alkyne-nitrone pair, an alkene-tetrazole pair, and an isonitrile-tetrazine pair.

In some embodiments, there is provided an anti-PD-L1 antibody agent (such as an isolated anti-PD-L1 antibody agent) comprising a) an antibody moiety comprising an anti-PD-L1 scFv fused to an Fc fragment, wherein the anti-PD-L1 scFv comprises: a V_{H} comprising a heavy chain complementarity determining region 1 (HC-CDR1) comprising the amino acid sequence of SEQ ID NO: 41, an HC-CDR2 comprising the amino acid sequence of SEQ ID NO: 42, and an HC-CDR3 comprising the amino acid sequence of SEQ ID NO: 43, or a variant thereof comprising up to a total of about 5 amino acid substitutions in the HC-CDRs; and a V_{L} comprising a light chain complementarity determining region 1 (LC-CDR1) comprising the amino acid sequence of SEQ ID NO: 44, an LC-CDR2 comprising the amino acid sequence of SEQ ID NO: 45, and an LC-CDR3 comprising the amino acid sequence of SEQ ID NO: 46, or a variant thereof comprising up to a total of about 5 amino acid substitutions in the LC-CDRs; and 2) a conjugation moiety that is capable of being conjugated to a second conjugation moiety in vivo. In some embodiments, the conjugation moiety comprises a member of a click chemistry pair. In some embodiments, the click chemistry pair is selected from the group consisting of a trans-cyclooctene (TCO)-tetrazine (Tz) pair, an azide-alkyne pair, an alkyne-nitrone pair, an alkene-tetrazole pair, and an isonitrile-tetrazine pair.

In some embodiments, there is provided an anti-PD-L1 antibody agent (such as an isolated anti-PD-L1 antibody agent) comprising a) an antibody moiety comprising: a V_{H} comprising the amino acid sequence of any one of SEQ ID NOs: 1, 5, 9, 11, and 13, or a variant thereof having at least about 80% (such as at least about any one of 80%, 85%, 90%, 95%, 96%, 97%, 98%, or 99%) sequence identity to the amino acid sequence of any one of SEQ ID NOs: 1, 5, 9, 11, and 13; and a V_{L} comprising the amino acid sequence of any one of SEQ ID NOs: 3, 7, 15, 17 and 19, or a variant thereof having at least about 80% (such as at least about any one of 80%, 85%, 90%, 95%, 96%, 97%, 98%, or 99%) sequence identity to the amino acid sequence of any one of SEQ ID NOs: 3, 7, 15, 17 and 19; and 2) a conjugation moiety that is capable of being conjugated to a second conjugation moiety in vivo. In some embodiments, the conjugation moiety comprises a member of a click chemistry pair. In some embodiments, the click chemistry pair is selected from the group consisting of a trans-cyclooctene (TCO)-tetrazine (Tz) pair, an azide-alkyne pair, an alkyne-nitrone pair, an alkene-tetrazole pair, and an isonitrile-tetrazine pair. In some embodiments, the anti-PD-L1 antibody moiety is humanized. In some embodiments, the anti-PD-L1 scFv comprises a first engineered cysteine residue at position 44 of V_{H} and a second engineered cysteine residue at position 100 of V_{L}, or a first engineered cysteine residue at position 105 of V_{H} and a second engineered cysteine residue at position 43 of V_{L}, wherein the first engineered cysteine residue and the second engineered cysteine residue form a disulfide bond, and wherein the amino acid positions are based on the Kabat numbering system. In some embodiments, the Fc fragment is an IgG1 Fc fragment. In some embodiments, the Fc fragment has H310A and H435Q mutations, wherein the amino acid positions are based on the Kabat numbering system. In some embodiments, the anti-PD-L1 scFv comprises the amino acid sequence of any one of SEQ ID NOs: 25, 27, 29, 31, 33, 35, 37 and 39, or a variant thereof having at least about 80% (such as at least about any one of 80%, 85%, 90%, 95%, 96%, 97%, 98%, or 99%) sequence identity to the amino acid sequence of any one of SEQ ID NOs: 25, 27, 29, 31, 33, 35, 37 and 39. In some embodiments, the antibody moiety comprises an amino acid sequence having at least about 80% sequence identity (such as at least about any one of 80%, 85%, 90%, 95%, 96%, 97%, 98%, or 99%) to the amino acid sequence of SEQ ID NO: 21 or 23. In some embodiments,

### Anti-PD-L1 Antibody Moieties

The anti-PD-L1 antibody agents described herein comprise an antibody moiety that specifically binds to PD-L1. Contemplated anti-PD-L1 antibody moieties include, for example, anti-PD-L1 scFv, anti-PD-L1 Fab, anti-PD-L1 Fc fusion protein (e.g., anti-PD-L1 scFv fused to an Fc). The anti-PD-L1 antibody moieties described herein include, but are not limited to, humanized antibodies, partially humanized antibodies, fully humanized antibodies, semi-synthetic antibodies, chimeric antibodies, mouse antibodies, human antibodies, and antibodies comprising the heavy chain and/or light chain CDRs discussed herein.

In some embodiments, the anti-PD-L1 antibody moiety specifically recognizes PD-L1. In some embodiments, the anti-PD-L1 antibody moiety specifically recognizes human PD-L1. In some embodiments, the anti-PD-L1 antibody moiety specifically recognizes the extracellular domain of PD-L1. In some embodiments, the anti-PD-L1 antibody moiety specifically recognizes an epitope within the amino acid sequence of amino acids 19-238 of SEQ ID NO: 49.

human PD-L1 sequence
SEQ ID NO: 49
MRIFAVFIFMTYWHLLNAFTVTVPKDLYVVEYGSNMTIECKFPVEKQLDL
AALIVYVVEMEDKNIIQFVHGEEDLKVQHSSYRQRARLLKDQLSLGNAAL
QITDVKLQDAGVYRCMISYGGADYKRITVKVNAPYNKINQRILVVDPVTS
EHELTCQAEGYPKAEVIWTSSDHQVLSGKTTTTNSKREEKLFNVTSTLRI
NTTTNEIFYCTFRRLDPEENHTAELVIPELPLAHPPNERTHLVILGAILL
CLGVALTFIFRLRKGRMMDVKKCGIQDTNSKKQSDTHLEET

In some embodiments, the anti-PD-L1 antibody moiety comprises: a heavy chain variable domain (V_{H}) comprising an HC-CDR3 comprising the amino acid sequence of SEQ ID NO: 43, or a variant thereof comprising up to about 5 (such as about any of 1, 2, 3, 4, or 5) amino acid substitutions; and ii) a light chain variable domain (V_{L}) comprising an LC-CDR3 comprising the amino acid sequence of SEQ ID NO: 46, or a variant thereof comprising up to about 5 (such as about any of 1, 2, 3, 4, or 5) amino acid substitutions.

In some embodiments, the anti-PD-L1 antibody moiety comprises: i) a V_{H} comprising an HC-CDR3 comprising the amino acid sequence of SEQ ID NO: 43; and ii) a V_{L} comprising an LC-CDR3 comprising the amino acid sequence of SEQ ID NO: 46.

In some embodiments, the anti-PD-L1 antibody moiety comprises: i) a V_{H} comprising an HC-CDR1 comprising the amino acid sequence of SEQ ID NO: 41, or a variant thereof comprising up to about 5 (such as about any of 1, 2, 3, 4, or 5) amino acid substitutions, an HC-CDR2 comprising the amino acid sequence of SEQ ID NO: 42, or a variant thereof comprising up to about 5 (such as about any of 1, 2, 3, 4, or 5) amino acid substitutions, and an HC-CDR3 comprising the amino acid sequence of a SEQ ID NO: 43, or a variant thereof comprising up to about 5 (such as about any of 1, 2, 3, 4, or 5) amino acid substitutions; and ii) a V_{L} comprising an LC-CDR1 comprising the amino acid sequence of SEQ ID NO: 44, or a variant thereof comprising up to about 5 (such as about any of 1, 2, 3, 4, or 5) amino acid substitutions, an LC-CDR2 comprising the amino acid sequence of SEQ ID NO: 45, or a variant thereof comprising up to about 5 (such as about any of 1, 2, 3, 4, or 5) amino acid substitutions, and an LC-CDR3 comprising the amino acid sequence of SEQ ID NO: 46, or a variant thereof comprising up to about 5 (such as about any of 1, 2, 3, 4, or 5) amino acid substitutions.

In some embodiments, the anti-PD-L1 antibody moiety comprises: i) a V_{H} comprising an HC-CDR1 comprising the amino acid sequence of SEQ ID NO: 41, an HC-CDR2 comprising the amino acid sequence of SEQ ID NO: 42, and an HC-CDR3 comprising the amino acid sequence of a SEQ ID NO: 43; or a variant thereof comprising up to about 5 (such as about any of 1, 2, 3, 4, or 5) amino acid substitutions in the HC-CDR sequences; and ii) a V_{L} comprising an LC-CDR1 comprising the amino acid sequence of SEQ ID NO: 44, an LC-CDR2 comprising the amino acid sequence of SEQ ID NO: 45, and an LC-CDR3 comprising the amino acid sequence of SEQ ID NO: 46; or a variant thereof comprising up to about 5 (such as about any of 1, 2, 3, 4, or 5) amino acid substitutions in the LC-CDR sequences.

In some embodiments, the anti-PD-L1 antibody moiety comprises: i) a V_{H} comprising an HC-CDR1 comprising the amino acid sequence of SEQ ID NO: 41, an HC-CDR2 comprising the amino acid sequence of SEQ ID NO: 42, and an HC-CDR3 comprising the amino acid sequence of a SEQ ID NO: 43; and ii) a V_{L} comprising an LC-CDR1 comprising the amino acid sequence of SEQ ID NO: 44, an LC-CDR2 comprising the amino acid sequence of SEQ ID NO: 45, and an LC-CDR3 comprising the amino acid sequence of SEQ ID NO: 46.

In some embodiments, the anti-PD-L1 antibody moiety comprises: i) a V_{H} comprising the amino acid sequences of SEQ ID NO: 41, SEQ ID NO: 42, and SEQ ID NO: 43; and ii) a V_{L} comprising the amino acid sequences of SEQ ID NO: 44, SEQ ID NO: 45, and SEQ ID NO: 46.

In some embodiments, the anti-PD-L1 antibody moiety comprises: i) a V_{H} comprising one, two or three CDRs of the VH comprising the amino acid sequence of SEQ ID NO: 1; and ii) a V_{L} comprising one, two or three CDRs of the VL comprising the amino acid sequence of SEQ ID NO: 3.

In some embodiments, the anti-PD-L1 antibody moiety comprises: a) a V_{H} comprising the amino acid sequence of SEQ ID NO: 1, or a variant thereof having at least about 80% (such as at least about any one of 80%, 85%, 90%, 95%, 96%, 97%, 98%, or 99%) sequence identity to SEQ ID NO: 1; and b) a V_{L} comprising the amino acid sequence of SEQ ID NO: 2, or a variant thereof having at least about 80% (such as at least about any one of 80%, 85%, 90%, 95%, 96%, 97%, 98%, or 99%) sequence identity to SEQ ID NO: 2. In some embodiment, the anti-PD-L1 antibody moiety comprises: a) a V_{H} comprising the amino acid sequence of SEQ ID NO: 1; and b) a V_{L} comprising the amino acid sequence of SEQ ID NO: 2.

In some embodiments, the anti-PD-L1 antibody is a chimeric antibody. In some embodiments, the anti-PD-L1 antibody moiety comprises mouse CDRs and human FR sequences. In some embodiments, the anti-PD-L1 antibody moiety comprises: a) a V_{H} comprising the amino acid sequence of SEQ ID NO: 5, or a variant thereof having at least about 80% (such as at least about any one of 80%, 85%, 90%, 95%, 96%, 97%, 98%, or 99%) sequence identity to SEQ ID NO: 5; and b) a V_{L} comprising the amino acid sequence of SEQ ID NO: 7, or a variant thereof having at least about 80% (such as at least about any one of 80%, 85%, 90%, 95%, 96%, 97%, 98%, or 99%) sequence identity to SEQ ID NO: 7. In some embodiment, the anti-PD-L1 antibody moiety comprises: a) a V_{H} comprising the amino acid sequence of SEQ ID NO: 5; and b) a V_{L} comprising the amino acid sequence of SEQ ID NO: 7.

In some embodiments, the anti-PD-L1 antibody is a humanized antibody. In some embodiments, the anti-PD-L1 antibody moiety comprises: a) a V_{H} comprising the amino acid sequence of any one of SEQ ID NOs: 9, 11 and 13, or a variant thereof having at least about 80% (such as at least about any one of 80%, 85%, 90%, 95%, 96%, 97%, 98%, or 99%) sequence identity to any one of SEQ ID NOs: 9, 11 and 13; and b) a V_{L} comprising the amino acid sequence of any one of SEQ ID NOs: 15, 17 and 19, or a variant thereof having at least about 80% (such as at least about any one of 80%, 85%, 90%, 95%, 96%, 97%, 98%, or 99%) sequence identity to any one of SEQ ID NOs: 15, 17 and 19. In some embodiments, the anti-PD-L1 antibody moiety comprises: a) a V_{H} comprising the amino acid sequence of any one of SEQ ID NOs: 9, 11 and 13; and b) a V_{L} comprising the amino acid sequence of any one of SEQ ID NOs: 15, 17 and 19.

In some embodiments, the anti-PD-L1 antibody moiety comprises: a) a V_{H} comprising the amino acid sequence of SEQ ID NO: 9, or a variant thereof having at least about 80% (such as at least about any one of 80%, 85%, 90%, 95%, 96%, 97%, 98%, or 99%) sequence identity to SEQ ID NO: 9; and b) a V_{L} comprising the amino acid sequence of SEQ ID NO: 15, or a variant thereof having at least about 80% (such as at least about any one of 80%, 85%, 90%, 95%, 96%, 97%, 98%, or 99%) sequence identity to SEQ ID NO: 15. In some embodiments, the anti-PD-L1 antibody moiety comprises: a) a V_{H} comprising the amino acid sequence of SEQ ID NO: 9; and b) a V_{L} comprising the amino acid sequence of SEQ ID NO: 15.

In some embodiments, the anti-PD-L1 antibody moiety comprises: a) a V_{H} comprising the amino acid sequence of SEQ ID NO: 11, or a variant thereof having at least about 80% (such as at least about any one of 80%, 85%, 90%, 95%, 96%, 97%, 98%, or 99%) sequence identity to SEQ ID NO: 11; and b) a V_{L} comprising the amino acid sequence of SEQ ID NO: 15, or a variant thereof having at least about 80% (such as at least about any one of 80%, 85%, 90%, 95%, 96%, 97%, 98%, or 99%) sequence identity to SEQ ID NO: 15. In some embodiments, the anti-PD-L1 antibody moiety comprises: a) a V_{H} comprising the amino acid sequence of SEQ ID NO: 11; and b) a V_{L} comprising the amino acid sequence of SEQ ID NO: 15.

In some embodiments, the anti-PD-L1 antibody moiety comprises: a) a V_{H} comprising the amino acid sequence of SEQ ID NO: 13, or a variant thereof having at least about 80% (such as at least about any one of 80%, 85%, 90%, 95%, 96%, 97%, 98%, or 99%) sequence identity to SEQ ID NO: 13; and b) a V_{L} comprising the amino acid sequence of SEQ ID NO: 15, or a variant thereof having at least about 80% (such as at least about any one of 80%, 85%, 90%, 95%, 96%, 97%, 98%, or 99%) sequence identity to SEQ ID NO: 15. In some embodiments, the anti-PD-L1 antibody moiety comprises: a) a V_{H} comprising the amino acid sequence of SEQ ID NO: 13; and b) a V_{L} comprising the amino acid sequence of SEQ ID NO: 15.

In some embodiments, the anti-PD-L1 antibody moiety comprises: a) a V_{H} comprising the amino acid sequence of SEQ ID NO: 9, or a variant thereof having at least about 80% (such as at least about any one of 80%, 85%, 90%, 95%, 96%, 97%, 98%, or 99%) sequence identity to SEQ ID NO: 9; and b) a V_{L} comprising the amino acid sequence of SEQ ID NO: 17, or a variant thereof having at least about 80% (such as at least about any one of 80%, 85%, 90%, 95%, 96%, 97%, 98%, or 99%) sequence identity to SEQ ID NO: 17. In some embodiments, the anti-PD-L1 antibody moiety comprises: a) a V_{H} comprising the amino acid sequence of SEQ ID NO: 9; and b) a V_{L} comprising the amino acid sequence of SEQ ID NO: 17.

In some embodiments, the anti-PD-L1 antibody moiety comprises: a) a V_{H} comprising the amino acid sequence of SEQ ID NO: 11, or a variant thereof having at least about 80% (such as at least about any one of 80%, 85%, 90%, 95%, 96%, 97%, 98%, or 99%) sequence identity to SEQ ID NO: 11; and b) a V_{L} comprising the amino acid sequence of SEQ ID NO: 17, or a variant thereof having at least about 80% (such as at least about any one of 80%, 85%, 90%, 95%, 96%, 97%, 98%, or 99%) sequence identity to SEQ ID NO: 17. In some embodiments, the anti-PD-L1 antibody moiety comprises: a) a V_{H} comprising the amino acid sequence of SEQ ID NO: 11; and b) a V_{L} comprising the amino acid sequence of SEQ ID NO: 17.

In some embodiments, the anti-PD-L1 antibody moiety comprises: a) a V_{H} comprising the amino acid sequence of SEQ ID NO: 13, or a variant thereof having at least about 80% (such as at least about any one of 80%, 85%, 90%, 95%, 96%, 97%, 98%, or 99%) sequence identity to SEQ ID NO: 13; and b) a V_{L} comprising the amino acid sequence of SEQ ID NO: 17, or a variant thereof having at least about 80% (such as at least about any one of 80%, 85%, 90%, 95%, 96%, 97%, 98%, or 99%) sequence identity to SEQ ID NO: 17. In some embodiments, the anti-PD-L1 antibody moiety comprises: a) a V_{H} comprising the amino acid sequence of SEQ ID NO: 13; and b) a V_{L} comprising the amino acid sequence of SEQ ID NO: 17.

In some embodiments, the anti-PD-L1 antibody moiety comprises: a) a V_{H} comprising the amino acid sequence of SEQ ID NO: 9, or a variant thereof having at least about 80% (such as at least about any one of 80%, 85%, 90%, 95%, 96%, 97%, 98%, or 99%) sequence identity to SEQ ID NO: 9; and b) a V_{L} comprising the amino acid sequence of SEQ ID NO: 19, or a variant thereof having at least about 80% (such as at least about any one of 80%, 85%, 90%, 95%, 96%, 97%, 98%, or 99%) sequence identity to SEQ ID NO: 19. In some embodiments, the anti-PD-L1 antibody moiety comprises: a) a V_{H} comprising the amino acid sequence of SEQ ID NO: 9; and b) a V_{L} comprising the amino acid sequence of SEQ ID NO: 19.

In some embodiments, the anti-PD-L1 antibody moiety comprises: a) a V_{H} comprising the amino acid sequence of SEQ ID NO: 11, or a variant thereof having at least about 80% (such as at least about any one of 80%, 85%, 90%, 95%, 96%, 97%, 98%, or 99%) sequence identity to SEQ ID NO: 11; and b) a V_{L} comprising the amino acid sequence of SEQ ID NO: 19, or a variant thereof having at least about 80% (such as at least about any one of 80%, 85%, 90%, 95%, 96%, 97%, 98%, or 99%) sequence identity to SEQ ID NO: 19. In some embodiments, the anti-PD-L1 antibody moiety comprises: a) a V_{H} comprising the amino acid sequence of SEQ ID NO: 11; and b) a V_{L} comprising the amino acid sequence of SEQ ID NO: 19.

In some embodiments, the anti-PD-L1 antibody moiety comprises: a) a V_{H} comprising the amino acid sequence of SEQ ID NO: 13, or a variant thereof having at least about 80% (such as at least about any one of 80%, 85%, 90%, 95%, 96%, 97%, 98%, or 99%) sequence identity to SEQ ID NO: 13; and b) a V_{L} comprising the amino acid sequence of SEQ ID NO: 19, or a variant thereof having at least about 80% (such as at least about any one of 80%, 85%, 90%, 95%, 96%, 97%, 98%, or 99%) sequence identity to SEQ ID NO: 19. In some embodiments, the anti-PD-L1 antibody moiety comprises: a) a V_{H} comprising the amino acid sequence of SEQ ID NO: 13; and b) a V_{L} comprising the amino acid sequence of SEQ ID NO: 19.

The heavy and light chain variable domains can be combined in various pair-wise combinations to generate a number of anti-nGPC3 antibody moieties. Exemplary anti-nGPC3 antibodies are provided in Table 3. The exemplary CDR, VH and VL sequences are delimited according to the INTERNATIONAL IMMUNOGENETICS INFORMATION SYSTEM® (IMGT). See, for example, Lefranc, M P et al., Nucleic Acids Res., 43:D413-422 (2015), the disclosure of which is incorporated herein by reference in its entirety. Those skilled in the art will recognize that many algorithms are known for prediction of CDR positions in antibody heavy chain and light chain variable regions, and antibody agents comprising CDRs from antibodies described herein, but are on prediction algorithms other than IMGT, are within the scope of this invention.

TABLE 3
Exemplary anti-PD-L1 antibody sequences.

| SEQ ID | | | |
|---|---|---|---|
| NO. | | | Amino acid sequence |
| AA | DNA | Description | (CDR sequences are underlined and bold) |
| 1 | 2 | VH | EVQLQQSGAELVKPGASVKLSCTASGFNIKDTYMYWVKQRPEQGLECIGRID |
| | | | PANDNTKYDPKFQGKATITADTSSNTAYVQLASLTSEDTAVYYCARAKNLLN |
| | | | YFDYWGQGTTLTVSS |
| 3 | 4 | VL | DIQMTQSPSSLSASLGERVTLSCRASQEISGYLSWLQQKPDGTIKRLIYATS |
| | | | TLDSGVPKRFSGSRSGSDYSLTISSLESEDFADYYCLQYAIYPLTFGAGTKL |
| | | | ELKR |
| 41 | | HC-CDR1 | GFNIKDTY |
| 42 | | HC-CDR2 | IDPANDNT |
| 43 | | HC-CDR3 | ARAKNLLNYFDY |
| 44 | | LC-CDR1 | QEISGY |
| 45 | | LC-CDR2 | ATS |
| 46 | | LC-CDR3 | LQYAIYPLT |
| 5 | 6 | Chimeric | EVQLQQSGAELVKPGASVKLSCTASGFNIKDTYMYWVKQRPEQGLECIGRID |
| | | VH 1 | PANDNTKYDPKFQGKATITADTSSNTAYVQLASLTSEDTAVYYCARAKNLLN |
| | | | YFDYWGQGTTLTVSSASTKGPSVFPLAPSSKSTSGGTAALGCLVKDYFPEPV |
| | | | TVSWNSGALTSGVHTFPAVLQSSGLYSLSSVVTVPSSSLGTQTYICNVNHKP |
| | | | SNTKVDKKVEPKSCDKTHTCPPCPAPELLGGPSVFLFPPKPKDTLMISRTPE |
| | | | VTCVVVDVSHEDPEVKFNWYVDGVEVHNAKTKPREEQYNSTYRVVSVLTVLH |
| | | | QDWLNGKEYKCKVSNKALPAPIEKTISKAKGQPREPQVYTLPPSRDELTKNQ |
| | | | VSLTCLVKGFYPSDIAVEWESNGQPENNYKTTPPVLDSDGSFFLYSKLTVDK |
| | | | SRWQQGNVFSCSVMHEALHNHYTQKSLSLSPG |
| 7 | 8 | Chimeric | DIQMTQSPSSLSASLGERVTLSCRASQEISGYLSWLQQKPDGTIKRLIYATS |
| | | VL1 | TLDSGVPKRFSGSRSGSDYSLTISSLESEDFADYYCLQYAIYPLTFGAGTKL |
| | | | ELKRTVAAPSVFIFPPSDEQLKSGTASVVCLLNNFYPREAKVQWKVDNALQS |
| | | | GNSQESVTEQDSKDSTYSLSSTLTLSKADYEKHKVYACEVTHQGLSSPVTKS |
| | | | FNRGEC |
| 9 | 10 | Humanized | QVQLVQSGAEVKKPGASVKVSCKASGFNIKDTYMYWVRQAPGQGLEWMGRID |
| | | VH 1 | PANDNTKYAQKFQGRVTITADTSTSTAYMELSSLRSEDTAVYYCARAKNLLN |
| | | | YFDYWGQGTLVTVSS |
| 11 | 12 | Humanized | QVQLVQSGAEVKKPGASVKVSCKASGFNIKDTYMYWVRQAPGQGLEWIGRID |
| | | VH 2 | PANDNTKYAPKFQGRVTITADTSTNTAYMELSSLRSEDTAVYYCARAKNLLN |
| | | | YFDYWGQGTLVTVSS |
| 13 | 14 | Humanized | EVQLVQSGAEVKKPGASVKVSCKASGFNIKDTYMYWVRQAPGQGLEWMGRID |
| | | VH3 | PANDNTKYAQKFQGRVTITADTSTNTAYMELSSLRSEDTAVYYCARAKNLLN |
| | | | YFDYWGQGTLVTVSS |
| 15 | 16 | Humanized | DIQMTQSPSSLSASVGDRVTITCRASQEISGYLSWYQQKPGKAPKRLIYATS |
| | | VL 1 | TLDSGVPSRFSGSGSGTDFTLTISSLQPEDFATYYCLQYAIYPLTFGQGTKL |
| | | | EIKR |
| 17 | 18 | Humanized | DIQMTQSPSSLSASVGDRVTITCRASQEISGYLSWLQQKPGKAPKRLIYATS |
| | | VL 2 | TLQSGVPSRFSGSRSGTDYTLTISSLQPEDFATYYCLQYAIYPLTFGQGTKL |
| | | | EIKR |
| 19 | 20 | Humanized | DIQMTQSPSSLSASVGDRVTITCRASQEISGYLSWYQQKPGKAPKRLIYATS |
| | | VL 3 | TLDSGVPSRFSGSRSGSDYTLTISSLQPEDFATYYCLQYAIYPLTFGQGTKL |
| | | | EIKR |

In some embodiments, the anti-PD-L1 antibody moiety competes for binding to a target PD-L1 with a second anti-PD-L1 antibody moiety according to any one of the anti-PD-L1 antibody moieties described herein. In some embodiments, the anti-PD-L1 antibody moiety binds to the same, or substantially the same, epitope as the second anti-PD-L1 antibody moiety. In some embodiments, binding of the anti-PD-L1 antibody moiety to the target PD-L1 inhibits binding of the second anti-PD-L1 antibody moiety to PD-L1 by at least about 70% (such as by at least about any one of 75%, 80%, 85%, 90%, 95%, 98% or 99%), or vice versa. In some embodiments, the anti-PD-L1 antibody moiety and the second anti-PD-L1 antibody moiety cross-compete for binding to the target PD-L1, i.e., each of the anti-PD-L1 antibody moieties competes with the other for binding to the target PD-L1.

Anti-PD-L1 scFv

In some embodiments, the anti-PD-L1 antibody moiety comprises an scFv. In some embodiments, the anti-PD-L1 antibody moiety is an scFv. In some embodiments, the anti-PD-L1 scFv has the configuration of (from N-terminus to C-terminus): V_{L}-L-V_{H}, or V_{H}-L-V_{L}, wherein L is a peptide linker. In some embodiments, the anti-PD-L1 scFv is chimeric, human, partially humanized, fully humanized, or semi-synthetic.

In some embodiments, the anti-PD-L1 scFv is engineered to have enhanced thermal stability. In some embodiments, the anti-PD-L1 scFv is engineered to have a melting temperature of about 55-70° C., such as about any one of 55-60, 60-65, or 65-70° C. In some embodiments, the anti-PD-L1 scFv comprises one or more (such as 1, 2, 3, or more) engineered disulfide bonds. In some embodiments, the anti-PD-L1 scFv comprises a first engineered cysteine residue at position 44 of V_{H} and a second engineered cysteine residue at position 100 of V_{L}, and/or a first engineered cysteine residue at position 105 of V_{H} and a second engineered cysteine residue at position 43 of V_{L}, wherein the first engineered cysteine residue and the second engineered cysteine residue form a disulfide bond, and wherein the amino acid positions are based on the Kabat numbering system. Other engineered disulfide bonds may be introduced into the anti-PD-L1 scFv by engineering a cysteine in the VH and a cysteine in the VL at suitable positions based on the structure and sequences of the scFv.

In some embodiments, the anti-PD-L1 scFv comprises: i) a V_{H} comprising an HC-CDR1 comprising the amino acid sequence of SEQ ID NO: 41, an HC-CDR2 comprising the amino acid sequence of SEQ ID NO: 42, and an HC-CDR3 comprising the amino acid sequence of a SEQ ID NO: 43; or a variant thereof comprising up to about 5 (such as about any of 1, 2, 3, 4, or 5) amino acid substitutions in the HC-CDR sequences; and ii) a V_{L} comprising an LC-CDR1 comprising the amino acid sequence of SEQ ID NO: 44, an LC-CDR2 comprising the amino acid sequence of SEQ ID NO: 45, and an LC-CDR3 comprising the amino acid sequence of SEQ ID NO: 46; or a variant thereof comprising up to about 5 (such as about any of 1, 2, 3, 4, or 5) amino acid substitutions in the LC-CDR sequences. In some embodiments, the anti-PD-L1 scFv is humanized. In some embodiments, the anti-PD-L1 scFv comprises from the N-terminus to the C-terminus: a V_{H}, an optional peptide linker, and a V_{L}. In some embodiments, the anti-PD-L1 scFv comprises from the N-terminus to the C-terminus: a V_{L}, an optional peptide linker, and a V_{H}. In some embodiments, the scFv comprises a peptide linker comprising the amino acid sequence of SEQ ID NO: 47 or 48. In some embodiments, the anti-PD-L1 scFv comprises one or more (such as 1, 2, 3, or more) engineered disulfide bonds. In some embodiments, the anti-PD-L1 scFv comprises a first engineered cysteine residue at position 44 of V_{H} and a second engineered cysteine residue at position 100 of V_{L}, and/or a first engineered cysteine residue at position 105 of V_{H} and a second engineered cysteine residue at position 43 of V_{L}, wherein the first engineered cysteine residue and the second engineered cysteine residue form a disulfide bond, and wherein the amino acid positions are based on the Kabat numbering system.

In some embodiments, the anti-PD-L1 scFv comprises: a V_{H} comprising the amino acid sequence of any one of SEQ ID NOs: 1, 5, 9, 11, and 13, or a variant thereof having at least about 80% (such as at least about any one of 80%, 85%, 90%, 95%, 96%, 97%, 98%, or 99%) sequence identity to the amino acid sequence of any one of SEQ ID NOs: 1, 5, 9, 11, and 13; and a V_{L} comprising the amino acid sequence of any one of SEQ ID NOs: 3, 7, 15, 17 and 19, or a variant thereof having at least about 80% (such as at least about any one of 80%, 85%, 90%, 95%, 96%, 97%, 98%, or 99%) sequence identity to the amino acid sequence of any one of SEQ ID NOs: 3, 7, 15, 17 and 19. In some embodiments, the anti-PD-L1 scFv is humanized. In some embodiments, the anti-PD-L1 scFv comprises from the N-terminus to the C-terminus: a V_{H}, an optional peptide linker, and a V_{L}. In some embodiments, the anti-PD-L1 scFv comprises from the N-terminus to the C-terminus: a V_{L}, an optional peptide linker, and a V_{H}. In some embodiments, the scFv comprises a peptide linker comprising the amino acid sequence of SEQ ID NO: 47 or 48. In some embodiments, the anti-PD-L1 scFv comprises one or more (such as 1, 2, 3, or more) engineered disulfide bonds. In some embodiments, the anti-PD-L1 scFv comprises a first engineered cysteine residue at position 44 of V_{H} and a second engineered cysteine residue at position 100 of V_{L}, and/or a first engineered cysteine residue at position 105 of V_{H} and a second engineered cysteine residue at position 43 of V_{L}, wherein the first engineered cysteine residue and the second engineered cysteine residue form a disulfide bond, and wherein the amino acid positions are based on the Kabat numbering system.

In some embodiments, the anti-PD-L1 scFv comprises the amino acid sequence of any one of SEQ ID NOs: 25, 27, 29, 31, 33, 35, 37 and 39, or a variant thereof having at least about 80% (such as at least about any one of 80%, 85%, 90%, 95%, 96%, 97%, 98%, or 99%) sequence identity to the amino acid sequence of any one of SEQ ID NOs: 25, 27, 29, 31, 33, 35, 37 and 39. In some embodiments, the anti-PD-L1 scFv comprises a His tag. In some embodiments, the anti-PD-L1 scFv comprises a His tag fused to the C-terminus of the anti-PD-L1 scFv moiety. In some embodiments, the anti-PD-L1 scFv comprises GGGGSHHHHHH (SEQ ID NO: 51). Exemplary anti-PD-L1 scFv sequences are shown in Table 4.

TABLE 4
Exemplary anti-PD-L1 scFv sequences.

| SEQ ID | | | |
|---|---|---|---|
| NO. | | | Amino acid Sequence |
| AA | DNA | Description | (CDR sequences are underlined and bold) |
| 25 | 26 | anti-human | QVQLVQSGAEVKKPGASVKVSCKASGFNIKDTYMYWVRQAPGQGLEWMGRID |
| | | PD-L1 scFv | PANDNTKYAQKFQGRVTITADTSTSTAYMELSSLRSEDTAVYYCARAKNLLN |
| | | variant 1 | YFDYWGQGTLVTVSSGGGGSGGGGSGGGGSGGGGSDIQMTQSPSSLSASVGD |
| | | | RVTITCRASQEISGYLSWLQQKPGKAPKRLIYATSTLQSGVPSRFSGSRSGT |
| | | | DYTLTISSLQPEDFATYYCLQYAIYPLTFGQGTKLEIKR |
| 27 | 28 | anti-human | QVQLVQSGAEVKKPGASVKVSCKASGFNIKDTYMYWVRQAPGQGLEWMGRID |
| | | PD-L1 scFv | PANDNTKYAQKFQGRVTITADTSTSTAYMELSSLRSEDTAVYYCARAKNLLN |
| | | variant 2 | YFDYWGQGTLVIVSSGSTSGSGKPGSGEGSTKGDIQMTQSPSSLSASVGDRV |
| | | | TITCRASQEISGYLSWLQQKPGKAPKRLIYATSTLQSGVPSRFSGSRSGTDY |
| | | | TLTISSLQPEDFATYYCLQYAIYPLTFGQGTKLEIKR |
| 29 | 30 | anti-human | QVQLVQSGAEVKKPGASVKVSCKASGFNIKDTYMYWVRQAPGQCLEWMGRID |
| | | PD-L1 scFv | PANDNTKYAQKFQGRVTITADTSTSTAYMELSSLRSEDTAVYYCARAKNLLN |
| | | variant 3 | YFDYWGQGTLVTVSSGGGGSGGGGSGGGGSGGGGSDIQMTQSPSSLSASVGD |
| | | | RVTITCRASQEISGYLSWLQQKPGKAPKRLIYATSTLQSGVPSRFSGSRSGT |
| | | | DYTLTISSLQPEDFATYYCLQYAIYPLTFGCGTKLEIKR |
| 31 | 32 | anti-human | QVQLVQSGAEVKKPGASVKVSCKASGFNIKDTYMYWVRQAPGQGLEWMGRID |
| | | PD-L1 scFv | PANDNTKYAQKFQGRVTITADTSTSTAYMELSSLRSEDTAVYYCARAKNLLN |
| | | variant 4 | YFDYWGCGTLVTVSSGGGGSGGGGSGGGGSGGGGSDIQMTQSPSSLSASVGD |
| | | | RVTITCRASQEISGYLSWLQQKPGKCPKRLIYATSTLQSGVPSRFSGSRSGT |
| | | | DYTLTISSLQPEDFATYYCLQYAIYPLTFGQGTKLEIKR |
| 33 | 34 | anti-human | DIQMTQSPSSLSASVGDRVTITCRASQEISGYLSWLQQKPGKAPKRLIYATS |
| | | PD-L1 scFv | TLQSGVPSRFSGSRSGTDYTLTISSLQPEDFATYYCLQYAIYPLTFGQGTKL |
| | | variant 5 | EIKRGGGGSGGGGSGGGGSGGGGSQVQLVQSGAEVKKPGASVKVSCKASGFN |
| | | | IKDTYMYWVRQAPGQGLEWMGRIDPANDNTKYAQKFQGRVTITADISISTAY |
| | | | MELSSLRSEDTAVYYCARAKNLLNYFDYWGQGTLVTVSS |
| 35 | 36 | anti-human | DIQMTQSPSSLSASVGDRVTITCRASQEISGYLSWLQQKPGKAPKRLIYATS |
| | | PD-L1 scFv | TLQSGVPSRFSGSRSGTDYTLTISSLQPEDFATYYCLQYAIYPLTFGQGTKL |
| | | variant 6 | EIKRGSTSGSGKPGSGEGSTKGQVQLVQSGAEVKKPGASVKVSCKASGFNIK |
| | | | DTYMYWVRQAPGQGLEWMGRIDPANDNTKYAQKFQGRVTITADTSTSTAYME |
| | | | LSSLRSEDTAVYYCARAKNLLNYFDYWGQGTLVTVSS |
| 37 | 38 | anti-human | DIQMTQSPSSLSASVGDRVTITCRASQEISGYLSWLQQKPGKCPKRLIYATS |
| | | PD-L1 scFv | TLQSGVPSRFSGSRSGTDYTLTISSLQPEDFATYYCLQYAIYPLTFGQGTKL |
| | | variant 7 | EIKRGGGGSGGGGSGGGGSGGGGSQVQLVQSGAEVKKPGASVKVSCKASGFN |
| | | | IKDTYMYWVRQAPGQGLEWMGRIDPANDNTKYAQKFQGRVTITADTSTSTAY |
| | | | MELSSLRSEDTAVYYCARAKNLLNYFDYWGCGTLVTVSS |
| 39 | 40 | anti-human | DIQMTQSPSSLSASVGDRVTITCRASQEISGYLSWLQQKPGKAPKRLIYATS |
| | | PD-L1 scFv | TLQSGVPSRFSGSRSGTDYTLTISSLQPEDFATYYCLQYAIYPLTFGCGTKL |
| | | variant 8 | EIKRGGGGSGGGGSGGGGSGGGGSQVQLVQSGAEVKKPGASVKVSCKASGFN |
| | | | IKDTYMYWVRQAPGQCLEWMGRIDPANDNTKYAQKFQGRVTITADISISTAY |
| | | | MELSSLRSEDTAVYYCARAKNLLNYFDYWGQGTLVTVSS |

Anti-PD-L1 scFv-Fc

In some embodiments, the anti-PD-L1 antibody moiety is an anti-PD-L1 scFv according to any one of the anti-PD-L1 scFvs described herein fused to an Fc fragment. In some embodiments, the ant-PD-L1 antibody moiety is fused to an Fc fragment via a peptide linker. The anti-PD-L1 antibody moiety may comprise any of the Fc fragments described in the “Antibody moieties” section above. In some embodiments, the Fc fragment is a human IgG1 Fc fragment. In some embodiments, the Fc fragment comprises one or more mutations to increase clearance or decrease half-life. For example, the Fc fragment may have H310A and/or H435Q mutations, wherein the amino acid positions are based on the Kabat numbering system.

In some embodiments, each chain of the Fc fragment is fused to the same entity. In some embodiments, the anti-PD-L1 scFv-Fc comprises two identical anti-PD-L1 scFvs described herein, each fused with one chain of the Fc fragment. In some embodiments, the anti-PD-L1 scFv-Fc is a homodimer. In some embodiments, the anti-PD-L1 scFv-Fc is a heterodimer.

In some embodiments, the anti-PD-L scFv-Fc comprises the amino acid sequence of SEQ ID NO: 21 or 23, or a variant thereof having at least about 8000 (such as at least about any one of 80%, 85%, 90%, 95%, 96%, 97%, 98%, or 99%) sequence identity to the amino acid sequence of SEQ ID NO: 21 or 23. Exemplary anti-PD-L1 scFv-Fc sequences are shown in Table 5.

TABLE 5
Exemplary anti-PD-L1 scFv sequences.

| SEQ ID | | | |
|---|---|---|---|
| NO. | | | Amino acid Sequence |
| AA | DNA | Description | (CDR sequences are underlined and bold) |
| 21 | 22 | hPD-L1 | QVQLVQSGAEVKKPGASVKVSCKASGFNIKDTYMYWVRQAPGQGLEWMGRIDP |
| | | scFv-hFc | ANDNTKYAQKFQGRVTITADTSTSTAYMELSSLRSEDTAVYYCARAKNLLNYF |
| | | wt | DYWGQGTLVTVSSGSTSGSGKPGSGEGSTKGDIQMTQSPSSLSASVGDRVTIT |
| | | | CRASQEISGYLSWLQQKPGKAPKRLIYATSTLQSGVPSRFSGSRSGTDYTLTI |
| | | | SSLQPEDFATYYCLQYAIYPLTFGQGTKLEIKRDKTHTCPPCPAPELLGGPSV |
| | | | FLFPPKPKDTLMISRTPEVTCVVVDVSHEDPEVKFNWYVDGVEVHNAKTKPRE |
| | | | EQYNSTYRVVSVLTVLHQDWLNGKEYKCKVSNKALPAPIEKTISKAKGQPREP |
| | | | QVYTLPPSRDELTKNQVSLTCLVKGFYPSDIAVEWESNGQPENNYKTTPPVLD |
| | | | SDGSFFLYSKLTVDKSRWQQGNVFSCSVMHEALHNHYTQKSLSLSPG |
| 23 | 24 | hPD-L1 | QVQLVQSGAEVKKPGASVKVSCKASGFNIKDTYMYWVRQAPGQGLEWMGRIDP |
| | | scFv-hFc | ANDNTKYAQKFQGRVTITADTSTSTAYMELSSLRSEDTAVYYCARAKNLLNYF |
| | | Mt | DYWGQGTLVTVSSGSTSGSGKPGSGEGSTKGDIQMTQSPSSLSASVGDRVTIT |
| | | | CRASQEISGYLSWLQQKPGKAPKRLIYATSTLQSGVPSRFSGSRSGTDYTLTI |
| | | | SSLQPEDFATYYCLQYAIYPLTFGQGTKLEIKRDKTHTCPPCPAPELLGGPSV |
| | | | FLFPPKPKDTLMISRTPEVTCVVVDVSHEDPEVKFNWYVDGVEVHNAKTKPRE |
| | | | EQYNSTYRVVSVLTVLAQDWLNGKEYKCKVSNKALPAPIEKTISKAKGQPREP |
| | | | QVYTLPPSRDELTKNQVSLTCLVKGFYPSDIAVEWESNGQPENNYKTTPPVLD |
| | | | SDGSFFLYSKLTVDKSRWQQGNVFSCSVMHEALHNQYTQKSLSLSPG |

### PD-1

### The PD-1/PD-L1 Pathway and Anti-PD Immunotherapy

The gene encoding programmed cell death 1 (PD-1) was first isolated from a murine T cell hybridoma and a hematopoietic progenitor cell line undergoing classic apoptosis in i992 (Ishida Y, et al. The EMBO journal 1992; 11:3887-3895). Structurally, as a CD28 and CTLA4 homologue, PD-1 is a type I transmembrane protein and belongs to the Ig superfamily (Sharpe A H and Freeman G J. Nature Reviews Immunology 2002; 2:116-126). The critical role of PD-1 in negatively modulating T cell responses and maintaining peripheral tolerance was shown by PD-1 gene ablation studies using different mouse models. PD-1-deficient mice in C57BL/6 background develop lupus-like autoimmune diseases due to enhanced proliferation of PD-1 deficient T cells against allogeneic antigen (Nishimura H et al. Immunity 1999; 11:141-151). In BALB/c, but not in immune-deficient BALB/c RAG2−/− background, PD-1 knockout mice develop dilated cardiomyopathy and suffered sudden death from congestive heart failure (Nishimura H et al. Science 2001; 291:319-322). One of the major contributing causes was later identified to be the generation of high-titer autoantibodies against the heart-specific protein cardiac troponin I (Okazaki T, et al. Nature medicine 2003; 9:1477-1483). In the NOD (non-obese diabetic) background, PD-1 deficiency leads to early onset of type I diabetes due to the accelerated islet-specific T cell expansion and infiltration into the pancreas islets (Yantha J et al. Diabetes 2010; 59:2588-2596). Overall, the PD-1 molecule acts as an inhibitory receptor involved in peripheral tolerance (Nishimura H, Honjo T. Trends in immunology 2001; 22:265-268). Unlike CTLA-4, the immunoreceptor tyrosine-based switch motif (ITSM) in PD-1, but not the nearby ITIM domain located in the cytoplasmic tail of PD-1, recruits SHP-2 phosphatase and reverses activation-induced phosphorylation after TCR signaling (Ishida Y, et al. The EMBO journal 1992; 11:3887-3895).

Several studies contributed to the discovery of the molecules that interact with PD-1. In 1999, the B7 homolog 1 (B7H1, also referred to as programmed death ligand-1 [PD-L1] in the later literature) was identified as a 290-amino-acid type I transmembrane glycoprotein belonging to the B7-CD28 family of the immunoglobulin superfamily that served as a negative regulator of human T cell response in vitro (Dong H et al. Nature medicine 1999; 5:1365-1369). One year later, it was shown that PD-L1 is a binding and functional counterpart of PD-1 (Freeman G J et al. The Journal of experimental medicine 2000; 192:1027-1034). It was later demonstrated that PD-L1 deficient mice were prone to autoimmune conditions (Dong H et al. Immunity 2004; 20:327-336). Notably, although the mRNAs for PD-1 and PD-L1 are expressed with broad spectrum in many cell types, both are inducible molecules as their expression patterns are strictly controlled by posttranscriptional regulation. PD-1 protein is not detectable on resting T cells, but is found on the cell surface within 24 hours of T-cell activation (Keir M E et al. Annual review of immunology 2008; 26:677-704). Under normal physiological conditions, PD-L1 protein is only expressed in the peripheral tissues, such as the tonsil, placenta, and a small fraction of macrophage-like cells in the lung and liver (Dong H et al. Nature medicine 2002; 8:793-800; Petroff M G et al. Placenta 2002; 23 Suppl A: 595-101). Extrinsic induction of PD-L1 is largely mediated by proinflammatory cytokines, such as interferon γ (IFN-γ), which indicates that PD-1/PD-L1 interaction plays an important role in the control of inflammatory response in the peripheral tissues (Zou W, Chen L. Nature reviews Immunology 2008; 8:467-477).

In addition to PD-L1, another PD-1 ligand called B7-DC (also known as PD-L2) was also independently identified by two laboratories (Tseng S Y et al. The Journal of experimental medicine 2001; 193:839-846; Latchman Y, et al. Nature immunology 2001; 2:261-268). PD-L2 was found to be selectively expressed on dendritic cells (DCs) and also negatively regulate T cell response by binding to PD-1. Recently, PD-L2 was also found to interact with repulsive guidance molecule family member b (RGMb), a molecule that is highly enriched in lung macrophages and may be required for induction of respiratory tolerance (Xiao Y, et al. The Journal of experimental medicine 2014; 211:943-959). Interestingly, PD-L1 was also found to have another receptor CD80 on activated T cells to deliver inhibitory signal, which may also contribute to the formation of T cell tolerance in vivo (Butte M J et al. Immunity 2007; 27:111-122; Park J J et al. Blood 2010; 116:1291-1298). Currently, at least five interacting molecules are known to be involved in the PD network. Thus, the original PD-1/PD-L1 pathway is renamed to the more suitable “PD pathway.” The presence of two ligands (PD-L1 and PD-L2) for PD-1 and two inhibitory receptors (PD-1 and CD80) for PD-L1 suggests that neither PD-1 blockade nor PD-L1 blockade would completely disrupt the PD pathway. Complete abrogation of the PD inhibitory pathway may require a combination strategy targeting both molecules.

The crucial function of the PD pathway in modulating the activity of T cells in the peripheral tissues in an inflammatory response to infection and in limiting autoimmunity appears to be hijacked by tumor cells and by viruses during chronic viral infections. PD-L1 is overexpressed on many freshly isolated human tumors from multiple tissue origins (Dong et al. Nature Medicine 2002; 8:793-800; Romano et al. Journal for Immunotherapy of Cancer 2015; 3:15; Hirano et al. Cancer Research 2005; 65:1089-1096). The expression of PD-L1 has been correlated with the progression and poor prognosis of certain types of human cancers (Wang et al. European journal of surgical oncology: the journal of the European Society of Surgical Oncology and the British Association of Surgical Oncology 2015; 41:450-456; Cierna et al. Annals of oncology: official journal of the European Society for Medical Oncology/ESMO 2016; 27:300-305; Gandini et al. Critical reviews in oncology/hematology 2016; 100:88-98; Thierauf et al. Melanoma research 2015; 25:503-509; Taube et al. Clinical cancer research: an official journal of the American Association for Cancer Research 2014; 20:5064-5074). During chronic viral infections, PD-L1 is persistently expressed on many tissues, while PD-1 is up-regulated on virus-specific CTLs (Yao et al. Trends in molecular medicine 2006; 12:244-246). Tumor- or virus-induced PD-L1 may utilize multiple mechanisms to facilitate the evasion of host immune surveillance, including T cell anergy, apoptosis, exhaustion, IL-10 production, DC suppression, as well as Treg induction and expansion (Zou et al. Nature reviews Immunology 2008; 8:467-477).

The PD pathway mediated escape of tumor immunity could be viewed as an “adaptive resistance” model (Chen et al. The Journal of clinical investigation 2015; 125:3384-3391; Yao et al. European journal of immunology 2013; 43:576-579). Specifically, activated tumor-specific T cells reach the tumor sites and become tumor-infiltrating lymphocytes (TILs). Upon recognition of the cognate antigen, TILs produce IFN-γ, which induces PD-L1 expression in many cell types in the tumor microenvironment, including DCs, macrophages, neutrophils, and B lymphocytes. Upon binding to PD-1, PD-L1 delivers a suppressive signal to T cells and an anti-apoptotic signal to tumor cells, leading to T cell dysfunction and tumor survival (Taube et al. Science translational medicine 2012; 4:127ra137; Spranger et al. Science translational medicine 2013; 5:200ra116). This model is not only supported by IHC-based observations that cell surface PD-L1 expression is detected only in cells that are adjacent to T cells, but also supported by studies showing a strong correlation between PD-L1 expression in tumor sites and the presence of TILs (Gandini et al. Critical reviews in oncology/hematology 2016; 100:88-98; Taube et al. Science translational medicine 2012; 4:127ra137), as well as the demonstration of IFN-γ as a major PD-L1 inducer in vivo in mouse tumor models (Dong et al. Nature medicine 2002; 8:793-800). The PD pathway mediated adaptive resistance hypothesis supports the observation that the majority of the PD-L1⁺ tumors can escape immune destruction even under strong anti-tumor immunity.

Based on the “adaptive resistance” model, immunotherapies targeting the PD pathway are designed to block the interaction of PD-1 and PD-L1, thus preventing the resistance to anti-tumor immunity. Even though the discovery of PD-1 did not lead to its application in anti-tumor therapies until the abundant expression of PD-L1 was discovered in tumors, linking the PD pathway with cancer treatment (Dong et al. Nature Medicine 1999; 5:1365-1369), by now, the FDA has already approved two PD-1 monoclonal antibodies for treating human cancers. These are OPDIVO® (also known as nivolumab, MDX-1106, BMS-936558 and ONO-4538) developed by Bristol-Myers Squibb (68), and KEYTRUDA® (also known as pembrolizumab, lambrolizumab and MK-3475) developed by Merck (69). Multiple monoclonal antibodies targeting either PD-1 or PD-L1 are under intense evaluations in hundreds of clinical trials involving thousands of patients. Anti-PD therapy has generated significant clinical benefits including remarkable regression of tumors and substantial extension of survival rate. Since anti-PD therapy targets tumor-induced immune defects through immune-modulation in the tumor sites, it offers durable efficacy, tolerable toxicity and a broad spectrum of cancer indications⁵⁹ The clinical success of anti-PD immunotherapy further validates the effectiveness of PD pathway blockade as a unique category of cancer therapy that is distinct from personalized or tumor type-specific therapies. By targeting tumors that have exploited defined immune checkpoint pathway to escape immune surveillance, anti-PD immunotherapy has taken center stage in immunotherapies for human cancers, and especially solid tumors.

### PD-L1 Expression at Tumor Site as a Biomarker to Predict Efficacy of Anti-PD Immunotherapy

Multiple solid tumor types show positive correlation between response rate to anti-PD immunotherapy and PD-L1 expression level within the tumor, including melanoma, RCC, NSCLC, colorectal cancer, as well as several hematologic malignancies, such as classical Hodgkin's lymphoma. In the melanoma clinical trials, PD-L1 overexpression detected by IHC was in approximately 45%-75% of the samples. In the nivolumab study, 45% of patients were positive for PD-L1 expression based on a 5% cutoff using the 28-8 antibody. The response rate for PD-L1-positive patients was 44%, compared to 17% for PD-L1-negative patients. PD-L1-positive melanoma patients treated with nivolumab had an OS of 21.1 months and a PFS of 9.1 months, as compared to 12.5 months and 2 months in PD-L1 negative patients, respectively. Pembrolizumab (anti-PD-1) has also been studied in advanced melanoma utilizing an IHC cutoff of 1%. PD-L1-positive patients (77%) had an ORR of 51%, while PD-L1-negative patients had an ORR of 6%. PD-L1-positive patients treated with pembrolizumab had a PFS of 12 months and a 1-year OS rate of 84%, while PD-L1-negative patients had a PFS of 3 months and a 1-year OS of 69%.

A similar trend was seen in NSCLC, where PD-L1-positive patients seemed to preferentially benefit from PD-1/PD-L1-directed therapy. Nivolumab was studied in patients with refractory NSCLC, and PD-L1 IHC was performed using a DAKO IHC assay with a 5% cutoff. On the basis of these criteria, 60% of patients were classified as positive for PD-L1, and the response rate in PD-L1-positive patients was 67% compared with 0% in PD-L1-negative patients. Pembrolizumab has also been investigated in NSCLC, utilizing a unique 50% IHC cutoff for PD-L1 expression using an unreported assay. On the basis of this cutoff, 25% of tumors were positive for PD-L1 and, at 6 months, PD-L1-positive patients had a 67% immune-related ORR (irORR), a 67% PFS rate, and a 89% OS rate compared to PD-L1-negative patients who had a 0% irORR, a 11% PFS rate, and a 33% OS rate. MPDL3280A, an anti-PD-L1 antibody, has also been studied in NSCLC utilizing a proprietary IHC platform with 0-3+ grading (3+ for >10% cells, 2+ for >5% cells, 0-1 for <5% cells). NSCLC patients with a PD-L1 expression score of 3+ had an 83% response rate, compared with 46% in patients with a score of either 2+ or 3+. Patients with 1+/2+/3+ PD-L1 expression had a 31% ORR.

PD-L1 IHC as a predictive biomarker has also been assessed in clinical trials involving multiple histologies. The nivolumab phase I study included patients with melanoma, RCC, NSCLC, metastatic colorectal cancer (mCRC), and metastatic castration-resistant prostate cancer (mCRPC). PD-L1 was detected by the 5H1 antibody utilizing a 5% threshold, and 60% of tumors were positive by this criterion. Patients with PD-L1-positive tumors had a 36% response rate, while patients with PD-L1 negative tumors had a 0% ORR. MPDL3280A has been studied in patients with melanoma, RCC, NSCLC, mCRC, and gastric cancer utilizing a proprietary PD-L1 IHC platform. PD-L1-positive patients had a 39% response rate, while PD-L1-negative patients had a 13% response rate.

Data from these clinical trials appears to suggest that patients with higher levels of PD-L1 according to IHC appeared to have superior responses to PD-1/PD-L1-directed therapy. However, relatively less is known about the nature of responses or survival outcomes in PD-L1-negative patients treated with anti-PD immunotherapy. Initial evaluations suggest that select PD-L1-negative patients with melanoma can still obtain durable responses to anti-PD-1/PD-L1 therapy, while response in PD-L1-negative NSCLC patients are rare. If this trend is reproduced in larger trials, it may represent a fundamental difference in the immunobiology between the two different tumor types, or it may represent a technical issue with IHC in different tissue types. In a phase I clinical trial for MPDL3280A in metastatic urothelial bladder cancer, PD-L1-positive patients had a 52% ORR at 12 weeks, compared with 11% in PD-L1-negative patients. Therefore, the depth and duration of responses in PD-L1-negative patients remain to be seen.

### V. Methods of Preparation and Compositions (e.g., Polynucleotide, Nucleic Acid Construct, Vector, Host Cell, Culture Medium)

In some embodiments, there is provided a method of preparing an antibody agent targeting an immune checkpoint ligand and/or a label compound (e.g., radionuclide compound) and a composition such as polynucleotide, nucleic acid construct, vector, host cell, culture medium that is produced during the preparation of the antibody agent targeting the immune checkpoint ligand and/or the label compound. The antibody agent and/or the label compound (e.g., radionuclide compound) described herein may be prepared by a number of processes as generally described below and more specifically in the Examples.

### Method of Producing an Antibody Agent

In some embodiments, there is provided a method of preparing an antibody agent targeting an immune checkpoint ligand, comprising: contacting a conjugation moiety with an antibody moiety, wherein the antibody moiety specifically binds to a immune checkpoint ligand (e.g., PD-L1 or PD-L1 like ligand), thereby providing the antibody agent. In some embodiments, the conjugation moiety comprises a member of a click chemistry pair. In some embodiments, the click chemistry pair is selected from the group consisting of an azide-alkyne pair, an alkyne-nitrone pair, an alkene and tetrazole pair, and an isonitrile (e.g., isocyanide) and tetrazine pair. In some embodiments, the conjugation moiety is a trans-cyclooctene (TCO) or a tetrazine (Tz). In some embodiments, the immune checkpoint ligand is PD-L1. In some embodiments, the immune checkpoint ligand is a PD-L1 like ligand. In some embodiments, the chelating compound is NOTA, DOTA or derivatives thereof. In some embodiments, the chelating compound is conjugated to a lysine of the antibody moiety. In some embodiments, the conjugation moiety comprises a functional group (e.g., N-hydroxysuccinimide (NHS) ester). In some embodiments, the conjugation moiety comprises TCO. In some embodiments, the conjugation moiety further comprises a spacer. In some embodiments, the spacer is between the conjugation moiety and the antibody agent. In some embodiments, the spacer is an alkyl spacer. In some embodiments, the spacer is a PEG spacer. In some embodiments, the PEG spacer comprises at least about 1, 2, 3, 4, 5, 6, 7, 8, 9, or 10 ethylene glycol unites. In some embodiments, the PEG spacer comprises about 1-10, 2-8, 3-6, or about 4 ethylene glycol units.

In some embodiments, there is provided a method of preparing an antibody agent targeting an immune checkpoint ligand, comprising: contacting a PD-L1 antibody moiety described herein with a conjugation moiety, thereby providing the antibody agent. In some embodiments, the conjugation moiety comprises a member of a click chemistry pair. In some embodiments, the click chemistry pair is selected from the group consisting of an azide-alkyne pair, an alkyne-nitrone pair, an alkene and tetrazole pair, and an isonitrile (e.g., isocyanide) and tetrazine pair. In some embodiments, the conjugation moiety is a trans-cyclooctene (TCO) or a tetrazine (Tz). In some embodiments, the chelating compound is NOTA, DOTA or derivatives thereof. In some embodiments, the chelating compound is conjugated to a lysine of the antibody moiety. In some embodiments, the conjugation moiety comprises a functional group (e.g., N-hydroxysuccinimide (NHS) ester). In some embodiments, the conjugation moiety comprises TCO. In some embodiments, the conjugation moiety further comprises a spacer. In some embodiments, the spacer is between the conjugation moiety and the antibody agent. In some embodiments, the spacer is an alkyl spacer. In some embodiments, the spacer is a PEG spacer. In some embodiments, the PEG spacer comprises at least about 1, 2, 3, 4, 5, 6, 7, 8, 9, or 10 ethylene glycol unites. In some embodiments, the PEG spacer comprises about 1-10, 2-8, 3-6, or about 4 ethylene glycol units.

In some embodiments, there is provided a method of preparing an antibody agent targeting an immune checkpoint ligand, comprising: contacting an antibody moiety (e.g., scFv) with a conjugation moiety, wherein the antibody moiety is an antibody or an antigen-binding fragment thereof, wherein the antibody or antigen-binding fragment thereof comprises a heavy chain variable region (VH) and a light chain variable region (VL), wherein: a) the VH comprises a heavy chain complementarity determining region 1 (HC-CDR1) comprising the amino acid sequence of SEQ ID NO: 41, an HC-CDR2 comprising the amino acid sequence of SEQ ID NO: 42, and an HC-CDR3 comprising the amino acid sequence of SEQ ID NO: 43, or a variant thereof comprising up to a total of about 5 amino acid substitutions in the HC-CDRs; and b) the VL comprises a light chain complementarity determining region 1 (LC-CDR1) comprising the amino acid sequence of SEQ ID NO: 44, an LC-CDR2 comprising the amino acid sequence of SEQ ID NO: 45, and an LC-CDR3 comprising the amino acid sequence of SEQ ID NO: 46, or a variant thereof comprising up to a total of about 5 amino acid substitutions in the LC-CDRs; and (b) contacting a conjugation moiety with the antibody moiety conjugate, thereby providing the antibody agent. In some embodiments, the conjugation moiety comprises a member of a click chemistry pair. In some embodiments, the click chemistry pair is selected from the group consisting of an azide-alkyne pair, an alkyne-nitrone pair, an alkene and tetrazole pair, and an isonitrile (e.g., isocyanide) and tetrazine pair. In some embodiments, the conjugation moiety is a trans-cyclooctene (TCO) or a tetrazine (Tz). In some embodiments, the immune checkpoint ligand is PD-L1. In some embodiments, the immune checkpoint ligand is a PD-L1 like ligand. In some embodiments, the chelating compound is NOTA, DOTA or derivatives thereof. In some embodiments, the chelating compound is conjugated to a lysine of the antibody moiety. In some embodiments, the conjugation moiety comprises a functional group (e.g., N-hydroxysuccinimide (NHS) ester). In some embodiments, the conjugation moiety comprises TCO. In some embodiments, the conjugation moiety further comprises a spacer. In some embodiments, the spacer is between the conjugation moiety and the antibody agent. In some embodiments, the spacer is an alkyl spacer. In some embodiments, the spacer is a PEG spacer. In some embodiments, the PEG spacer comprises at least about 1, 2, 3, 4, 5, 6, 7, 8, 9, or 10 ethylene glycol unites. In some embodiments, the PEG spacer comprises about 1-10, 2-8, 3-6, or about 4 ethylene glycol units.

### Method of Producing a Label Compound

In some embodiments, there is provided a method of preparing a label compound (e.g., a radionuclide compound) comprising: (a) conjugating a chelating compound to any one of the conjugation moiety described herein to provide an conjugation moiety conjugate; and (b) contacting a label (e.g., radionuclide) with the conjugation moiety conjugate, thereby providing the label compound. In some embodiments, the chelating compound is NOTA, DOTA or derivatives thereof. In some embodiments, the radionuclide is selected from the group consisting of ⁶⁴Cu, ¹⁸F, ⁶⁷Ga, ⁶⁸Ga, ¹¹¹In, ¹⁷⁷Lu, ⁹⁰Y, ⁸⁹Zr, ⁶¹Cu, ⁶²Cu, ⁶⁷Cu, ¹⁹F, ⁶⁶Ga, ⁷²Ga, ⁴⁴Sc, ⁴⁷Sc, ⁸⁶Y, ⁸⁸Y and ⁴⁵Ti. In some embodiments, the radionuclide is ⁶⁸Ga. In some embodiments, the conjugation moiety can be conjugated to a second conjugation moiety in vivo via click chemistry. In some embodiments, the conjugation moiety comprises TCO or Tz. In some embodiments, the conjugation moiety comprises Tz. In some embodiments, the conjugation moiety further comprises a spacer. In some embodiments, the spacer is selected from an alkyl spacer and a PEG spacer. In some embodiments, each conjugation moiety (e.g., each tetrazine) is ligated with at least 1, 2, 3, 4, 5, 6, 7, 8, 9, or 10 PEG spacers. In some embodiments, each conjugation moiety (e.g., each tetrazine) is ligated with about 1-20, 5-15, 8-12, or about 10 PEG spacers.

In some embodiments, there is provided a method of preparing a label compound (e.g., a radionuclide compound) comprising: (a) contacting a chelating compound with a label (e.g., a radionuclide); (b) conjugating the chelating compound that chelates the label (e.g., radionuclide) to any one of the conjugation moieties described herein, thereby providing the label compound. In some embodiments, the chelating compound is NOTA, DOTA or derivatives thereof. In some embodiments, the radionuclide is selected from the group consisting of ⁶⁴Cu, ¹⁸F, ⁶⁷Ga, ⁶⁸Ga, ¹¹¹In, ¹⁷⁷Lu, ⁹⁰Y, ⁸⁹Zr, ⁶¹Cu, ⁶²Cu, ⁶⁷Cu, ¹⁹F, ⁶⁶Ga, ⁷²Ga, ⁴⁴Sc, ⁴⁷Sc, ⁸⁶Y, ⁸⁸Y and ⁴⁵Ti. In some embodiments, the radionuclide is ⁶⁸Ga. In some embodiments, the conjugation moiety can be conjugated to a second conjugation moiety in vivo via click chemistry. In some embodiments, the conjugation moiety comprises TCO or Tz. In some embodiments, the conjugation moiety comprises Tz. In some embodiments, the conjugation moiety further comprises a spacer. In some embodiments, the spacer is selected from an alkyl spacer and a PEG spacer. In some embodiments, each conjugation moiety (e.g., each tetrazine) is ligated with at least 1, 2, 3, 4, 5, 6, 7, 8, 9, or 10 PEG spacers. In some embodiments, each conjugation moiety (e.g., each tetrazine) is ligated with about 1-20, 5-15, 8-12, or about 10 PEG spacers.

In some embodiments, there is provided method of preparing a radionuclide compound comprising: a) (a) conjugating a chelating compound to a conjugation moiety to provide an conjugation moiety conjugate, wherein the chelating compound is DOTA, and wherein the conjugation moiety comprises Tz; b) contacting ⁶⁸Ga with the conjugation the Tz moiety conjugate, thereby providing the radionuclide compound. In some embodiments, the conjugation moiety further comprises a spacer. In some embodiments, the spacer is selected from an alkyl spacer and a PEG spacer. In some embodiments, each conjugation moiety (e.g., each tetrazine) is ligated with at least 1, 2, 3, 4, 5, 6, 7, 8, 9, or 10 PEG spacers. In some embodiments, each conjugation moiety (e.g., each tetrazine) is ligated with about 1-20, 5-15, 8-12, or about 10 PEG spacers.

### Label Compound (e.g., Radionuclide Compound)

One aspect of the present application provides a label compound (e.g., a radionuclide compound) comprising a label (e.g., a radionuclide) and a conjugation moiety as described herein. Any one of the label compounds described in this section may be used in the methods of determining the distribution and/or expression level of an immune checkpoint ligand, or methods of diagnosis or treatment described herein.

In some embodiments, the radionuclide is selected from the group consisting of ⁶⁴Cu, ¹⁸F, ⁶⁷Ga, ⁶⁸Ga, ¹¹¹In, ¹⁷⁷Lu, ⁹⁰Y, ⁸⁹Zr, ⁶¹Cu, ⁶²Cu, ⁶⁷Cu, ¹⁹F, ⁶⁶Ga, ⁷²Ga, ⁴⁴Sc, ⁴⁷Sc, ⁸⁶Y, ⁸⁸Y and ⁴⁵Ti. In some embodiments, the radionuclide is ⁶⁸Ga. In some embodiments, the radionuclide compound comprises a chelating compound that chelates the radionuclide. In some embodiments, the chelating compound is 1,4,7-triazacyclononane-1,4,7-trisacetic acid (NOTA), 1, 4, 7, 10-tetraazacyclododecane-1,4,7,10-tetraacetic acid (DOTA) or derivatives thereof. In some embodiments, the chelating compound is NOTA. In some embodiments, the conjugation moiety can be conjugated to a second conjugation moiety in vivo. In some embodiments, the conjugation moiety can be conjugated to a second conjugation moiety in vivo via click chemistry. In some embodiments, the click chemistry is based upon an inverse-electron-demand Diels-Alder cycloaddition, wherein the click chemistry involves a coupling reaction between a strained alkene and a tetrazine. In some embodiments, the conjugation moiety in the radionuclide compound comprises or is a trans-cyclooctene (TCO) or a tetrazine (Tz). In some embodiments, the conjugation moiety in the radionuclide compound comprises or is a Tz. the Tz is 6-Methyl-substituted tetrazine (6-Me tetrazine) or 6-hydrogen-substituted tetrazine (6-H tetrazine). In some embodiments, the spacer is between the conjugation moiety and the label. In some embodiments, the spacer is an alkyl spacer. In some embodiments, the spacer is a PEG spacer. In some embodiments, the PEG spacer comprises at least about 1, 2, 3, 4, 5, 6, 7, 8, 9, or 10 ethylene glycol unites. In some embodiments, the PEG spacer comprises about 1-10, 2-8, 3-6, or about 4 ethylene glycol units.

### Radionuclide

The label compound described herein comprises a label. For diagnostic purposes, the label may be a radionuclide, a radiological contrast agent, a paramagnetic ion, a metal, a fluorescent label, a chemiluminescent label, an ultrasound contrast agent and a photoactive agent. Such diagnostic labels are well known and any such known labels may be used.

In some embodiments, the label compound comprises a radionuclide. “Radionuclides” are often referred to as “radioactive isotopes” or “radioisotopes.” Exemplary radionuclides or stable isotopes that may be attached to the antibody moieties described herein include, but are not limited to, ¹¹⁰In, ¹¹¹In, ¹⁷⁷Lu, ¹⁸F, ⁵²Fe, ⁶²Cu, ⁶⁴Cu, ⁶⁷Cu, ⁶⁷Ga, ⁶⁸Ga, ⁸⁶Y, ⁹⁰Y, ⁸⁹Zr, ^{94m}Tc, ⁹⁴Tc, ^{99m}Tc, ¹²⁰I, ¹²³I, ¹²⁴I, ¹²⁵I, ¹³¹I, ¹⁵⁴⁻¹⁵⁸Gd, ³²P, ¹¹C, ¹³N, ¹⁵O, ¹⁸⁶Re, ¹⁸⁸Re, ⁵¹Mn, ^{52m}Mn, ⁵⁵Co, ⁷²As, ⁷⁵Br, ⁷⁶Br, ^{82m}Rb, ⁸³Sr, or other gamma-, beta-, or positron-emitters. In some embodiments, the radionuclide is selected from the group consisting of ⁶⁴Cu, ¹⁸F, ⁶⁷Ga, ⁶⁸Ga, ¹¹¹In, ¹⁷⁷Lu, ⁹⁰Y, ⁸⁹Zr, ⁶¹Cu, ⁶²Cu, ⁶⁷Cu, ¹⁹F, ⁶⁶Ga, ⁷²Ga, ⁴⁴Sc, ⁴⁷Sc, ⁸⁶Y, ⁸⁸Y and ⁴⁵Ti. In some embodiments, the radionuclide is ⁶⁸Ga.

Paramagnetic ions of use may include chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) or erbium (III). Metal contrast agents may include lanthanum (III), gold (III), lead (II) or bismuth (III). Radiopaque diagnostic agents may be selected from compounds, barium compounds, gallium compounds, and thallium compounds. A wide variety of fluorescent labels are known in the art, including but not limited to fluorescein isothiocyanate, rhodamine, phycoelytherin, phycocyanin, allophycocyanin, ophthaldehyde and fluorescamine. Chemiluminescent labels of use may include luminol, isoluminol, an aromatic acridinium ester, an imidazole, an acridinium salt or an oxalate ester.

Radioimmunodetection (RAID) has emerged as a clinically useful field over the last 35 years. Almost 1000 clinical trials using RAID have been conducted during this time, with some clear and important findings. The greater facility of this technique to detect lesions deemed “occult” by conventional imaging was recognized even in early studies and has repeatedly been confirmed by studies, regardless of antibody, tumor or radionuclide type.

Many radionuclides, such as ⁶⁸Ga, ⁹⁹Tc, ⁶⁴Cu and ¹⁸F are good imaging agent of choice. They usually have a gamma or beta energy that is ideal for safe imaging, and are inexpensive and are readily available, being generator-produced and carrier-free. Their short half-life (less than 6 hrs) readily lends themselves to coupling with antibody fragments for early imaging studies.

In some embodiments, the label compound (e.g., radionuclide compound) comprises a chelating compound that chelates the label (e.g., the radionuclide). In some embodiments, the chelating compound chelates a radioactive metal. In some embodiments, the chelating compound chelates a metal ¹⁸F. In some embodiments, the chelating compound is a hydrophilic chelating compound, which can bind metal ions and help to ensure rapid in vivo clearance. Suitable chelating compounds may be selected for their particular metal-binding properties, and substitution by known chemical cross-linking techniques or by use of chelators with side-chain reactive groups (such as bifunctional chelating compounds) may be performed with only routine experimentation.

Particularly useful metal-chelating compound combinations include 2-benzyl-DTPA (diethylenetriamine pentaacetic acid) and its monomethyl and cyclohexyl analogs, used with diagnostic isotopes in the general energy range of 60 to 4,000 keV, such as ¹²⁵I, ¹³¹I, ¹²³I, ¹²⁴I, ⁶²Cu, ⁶⁴Cu, ¹⁸F, ¹¹¹In, ⁶⁷Ga, ⁶⁸Ga, ⁹⁹Tc, ⁹⁴Tc, ¹¹C, ¹³N, ¹⁵O, ⁷⁶Br, for radio-imaging. The same chelating compounds, when complexed with nonradioactive metals, such as manganese, iron and gadolinium are useful for MRI. Macrocyclic chelating compounds such as NOTA (1,4,7-triazacyclononane-1,4,7-triacetic acid), TETA (bromoacetamido-benzyl-tetraethylaminetetraacetic acid) and NETA ({4-[2-(bis-carboxymethyl-amino)-ethyl]-7-carboxymethyl-[1,4,7]triazonan-1-yl}-acetic acid) are of use with a variety of diagnostic radiometals, such as gallium, yttrium and copper. Such metal-chelating complexes can be made very stable by tailoring the ring size to the metal of interest. The person of ordinary skill will understand that, by varying the groups attached to a macrocyclic ring structure such as NOTA, the binding characteristics and affinity for different metals and/or radionuclides may change and such derivatives or analogs of, e.g. NOTA, may therefore be designed to bind any of the metals or radionuclides discussed herein.

DTPA and DOTA-type chelators, where the ligand includes hard base chelating functions such as carboxylate or amine groups, are most effective for chelating hard acid cations, especially Group IIa and Group IIIa metal cations. Such metal-chelate complexes can be made very stable by tailoring the ring size to the metal of interest. Other ring-type chelators such as macrocyclic polyethers are of interest for stably binding nuclides. Porphyrin chelators may be used with numerous metal complexes. More than one type of chelator may be conjugated to a peptide to bind multiple metal ions. Chelators such as those disclosed in U.S. Pat. No. 5,753,206, especially thiosemicarbazonylglyoxylcysteine (Tseg-Cys) and thiosemicarbazinyl-acetylcysteine (Tsca-Cys) chelators are advantageously used to bind soft acid cations of Tc, Re, Bi and other transition metals, lanthanides and actinides that are tightly bound to soft base ligands. Other hard acid chelators such as DOTA, TETA and the like can be substituted for the DTPA and/or TscgCys groups.

In some embodiments, the chelating compound comprises a functional group that can be conjugated to the antibody moiety. In some embodiments, the chelating compound comprises a functional group that is reactive with a primary amine (—NH₂) group in the antibody moiety. Primary amines exist at the N-terminus of each polypeptide chain and in the side-chain of lysine (Lys) amino acid residues. Exemplary functional groups that can be conjugated to a primary amine, e.g., a lysine side chain, of the antibody moiety, include, but are not limited to, isothiocyanates, isocyanates, acyl azides, N-hydroxysuccinimide (NHS) esters, sulfonyl chlorides, aldehydes, glyoxals, epoxides, oxiranes, carbonates, aryl halides, imidoesters, carbodiimides, anhydrides, and fluorophenyl esters. Most of these functional groups conjugate to amines by either acylation or alkylation.

In some embodiments, the chelating compound comprises a functional group that is reactive with a cysteine side chain (i.e., sulfhydryl group) in the antibody moiety. Exemplary sulfhydryl reactive groups include, but are not limited to, haloacetyls, maleimides, aziridines, acryloyls, arylating agents, vinylsulfones, pyridyl disulfides, TNB-thiols and disulfide reducing agents. Most of these groups conjugate to sulfhydryls by either alkylation (usually the formation of a thioether bond) or disulfide exchange (formation of a disulfide bond).

In some embodiments, the chelating compound is NOTA, including NOTA derivatives. In some embodiments, the chelating compound (e.g., NOTA compound) comprise functional groups suitable for conjugation to antibody moieties, e.g., via amino acid side chains such as lysines and cysteines. In some embodiments, the imaging agent comprises NOTA conjugated to the antibody moiety. In some embodiments, the NOTA compound comprises an isothiocyanate (—SCN) group. In some embodiments, the NOTA compound is p-SCN-Bn-NOTA. In some embodiments, the chelating compound comprises a NOTA conjugated to a lysine residue in the antibody moiety, and the NOTA chelates ⁶⁸Ga. In some embodiments, the NOTA compound is first labeled with a radioactive metal, such as ⁶⁸Ga, or ¹⁸F-metal, and then conjugated to the antibody moiety.

### Conjugation Moiety

One aspect of the present application provides conjugation moieties that can be incorporated in the antibody agent and/or the label compound. In some embodiments, the conjugation moiety incorporated in the antibody agent (e.g., a first conjugation moiety) is capable of being conjugated to the conjugation moiety (e.g., a second conjugation moiety) incorporated in the label compound (e.g., radionuclide compound) in vivo.

In some embodiments the conjugation of the first conjugation moiety and second conjugation moiety is non-covalent. In some embodiments, the conjugation of the first conjugation moiety and second conjugation moiety is covalent.

In some embodiments, the first conjugation moiety and/or the second conjugation moiety each comprises a nucleic acid which are complementary to each other. In some embodiments, the nucleic acid is a DNA. In some embodiments, the first conjugation moiety and the second conjugation moiety are conjugated to each other via DNA-DNA hybrization.

In some embodiments, the first conjugation moiety and the second conjugation moiety are conjugated via a covalent bond between the two conjugation moieties. In some embodiments, the first conjugation moiety or the second conjugation moiety comprises a cysteine residue, a lysine residue, or a tyrosine residue. In some embodiments, the first conjugation moiety and the second conjugation moiety are conjugated based upon a coupling of cysteine residues (e.g., via a disulfide bond).

In some embodiments, the first conjugation moiety and the second conjugation moiety are conjugated based upon an antibody-drug conjugate.

### Click Chemistry

In some embodiments, the first conjugation moiety and the second conjugation moiety each comprises a member of a click chemistry pair, and are conjugated to each other via click chemistry. The click chemistry pair described herein is two chemical moieties that are capable of exclusively reacting with each other via click chemistry.

In some embodiments, the click chemistry pair is selected from the group consisting of an azide-alkyne pair, an alkyne-nitrone pair, an alkene and tetrazole pair, and an isonitrile (e.g., isocyanide) and tetrazine pair.

In some embodiments, the click chemistry is based upon an azide-alkyne Huisgen cycloaddition. In some embodiments, the azide-alkyne Huisgen cycloaddition is copper catalyzed. In some embodiments, the azide-alkyne Huisgen cycloaddition is copper-free. In some embodiments, the conjugation moiety comprises a cyclic derivative of the alkynyl group. In some embodiments, the cyclic derivative of the alkynyl group is selected from cyclooctyne, difluorinated cyclooctyne, and dibenzocyclooctyne. In some embodiments, the click chemistry is based upon strain promoted Huisgen cycloaddition of azides. In some embodiments, the click chemistry is based upon reaction of strained alkenes.

In some embodiments, the click chemistry is based upon Staudinger-Bertozzi ligation, wherein the click chemistry involved a coupling reaction between an azide and a phosphine.

In some embodiments, the click chemistry is based upon an inverse-electron-demand Diels-Alder cycloaddition, wherein the click chemistry involves a coupling reaction between a strained alkene and a tetrazine. In some embodiments, a conjugation moiety comprises or is a trans-cyclooctene (TCO) or a tetrazine (Tz). In some embodiments, the first conjugation moiety comprises or is a trans-cyclooctene (TCO) and the second conjugation moiety comprises or is a tetrazine (Tz). In some embodiments, the first conjugation moiety comprises or is a Tz and the second conjugation moiety comprises or is a TCO. In some embodiments, the Tz is 6-Methyl-substituted tetrazine (6-Me tetrazine). In some embodiments, the Tz is 6-hydrogen-substituted tetrazine (6-H tetrazine).

In some embodiments, the conjugation moiety (e.g., tetrazine) further comprises with a spacer. In some embodiments, the spacer is between the conjugation moiety and the antibody moiety. In some embodiments, the spacer is between the conjugation moiety and the label. In some embodiments, the spacer is an alkyl spacer. In some embodiments, the spacer is a PEG spacer. In some embodiments, the PEG spacer comprises at least about 1, 2, 3, 4, 5, 6, 7, 8, 9, or 10 ethylene glycol unites. In some embodiments, the PEG spacer comprises about 1-20, 5-15, 8-12, or about 10 ethylene glycol units.

### Antibody Expression and Production

The antibody moieties described herein can be prepared using any known methods in the art, including those described below and in the Examples.

### Monoclonal Antibodies

Monoclonal antibodies are obtained from a population of substantially homogeneous antibodies, i.e., the individual antibodies comprising the population are identical except for possible naturally occurring mutations and/or post-translational modifications (e.g., isomerizations, amidations) that may be present in minor amounts. Thus, the modifier “monoclonal” indicates the character of the antibody as not being a mixture of discrete antibodies. For example, the monoclonal antibodies may be made using the hybridoma method first described by Kohler et al., Nature, 256:495 (1975), or may be made by recombinant DNA methods (U.S. Pat. No. 4,816,567). In the hybridoma method, a mouse or other appropriate host animal, such as a hamster or a llama, is immunized as hereinabove described to elicit lymphocytes that produce or are capable of producing antibodies that will specifically bind the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986). Also see Example 1 for immunization in Camels.

The immunizing agent will typically include the antigenic protein or a fusion variant thereof. Generally either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell. Goding, Monoclonal Antibodies: Principles and Practice, Academic Press (1986), pp. 59-103.

Immortalized cell lines are usually transformed mammalian cells, particularly myeloma cells of rodent, bovine and human origin. Usually, rat or mouse myeloma cell lines are employed. The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which are substances that prevent the growth of HGPRT-deficient cells.

Preferred immortalized myeloma cells are those that fuse efficiently, support stable high-level production of antibody by the selected antibody-producing cells, and are sensitive to a medium such as HAT medium. Among these, preferred are murine myeloma lines, such as those derived from MOPC-21 and MPC-11 mouse tumors available from the Salk Institute Cell Distribution Center, San Diego, Calif. USA, and SP-2 cells (and derivatives thereof, e.g., X63-Ag8-653) available from the American Type Culture Collection, Manassas, Va. USA. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunosorbent assay (ELISA).

The culture medium in which the hybridoma cells are cultured can be assayed for the presence of monoclonal antibodies directed against the desired antigen. Preferably, the binding affinity and specificity of the monoclonal antibody can be determined by immunoprecipitation or by an in vitro binding assay, such as radioimmunoassay (RIA) or enzyme-linked assay (ELISA). Such techniques and assays are known in the in art. For example, binding affinity may be determined by the Scatchard analysis of Munson et al., Anal. Biochem., 107:220 (1980).

After hybridoma cells are identified that produce antibodies of the desired specificity, affinity, and/or activity, the clones may be subcloned by limiting dilution procedures and grown by standard methods (Goding, supra). Suitable culture media for this purpose include, for example, D-MEM or RPMI-1640 medium. In addition, the hybridoma cells may be grown in vivo as tumors in a mammal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

Monoclonal antibodies may also be made by recombinant DNA methods, such as those described in U.S. Pat. No. 4,816,567, and as described above. DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese hamster ovary (CHO) cells, or myeloma cells that do not otherwise produce immunoglobulin protein, in order to synthesize monoclonal antibodies in such recombinant host cells. Review articles on recombinant expression in bacteria of DNA encoding the antibody include Skerra et al., Curr. Opinion in Immunol., 5:256-262 (1993) and Plückthun, Immunol. Revs. 130:151-188 (1992).

In a further embodiment, antibodies can be isolated from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991) describe the isolation of murine and human antibodies, respectively, using phage libraries. Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nucl. Acids Res., 21:2265-2266 (1993)). Thus, these techniques are viable alternatives to traditional monoclonal antibody hybridoma techniques for isolation of monoclonal antibodies.

The DNA also may be modified, for example, by substituting the coding sequence for human heavy- and light-chain constant domains in place of the homologous murine sequences (U.S. Pat. No. 4,816,567; Morrison, et al., Proc. Natl Acad. Sci. USA, 81:6851 (1984)), or by covalently joining to the immunoglobulin coding sequence all or part of the coding sequence for a non-immunoglobulin polypeptide. Typically such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

The monoclonal antibodies described herein may by monovalent, the preparation of which is well known in the art. For example, one method involves recombinant expression of immunoglobulin light chain and a modified heavy chain. The heavy chain is truncated generally at any point in the Fc region so as to prevent heavy chain crosslinking. Alternatively, the relevant cysteine residues may be substituted with another amino acid residue or are deleted so as to prevent crosslinking. In vitro methods are also suitable for preparing monovalent antibodies. Digestion of antibodies to produce fragments thereof, particularly Fab fragments, can be accomplished using routine techniques known in the art.

Chimeric or hybrid antibodies also may be prepared in vitro using known methods in synthetic protein chemistry, including those involving crosslinking agents. For example, immunotoxins may be constructed using a disulfide-exchange reaction or by forming a thioether bond. Examples of suitable reagents for this purpose include iminothiolate and methyl-4-mercaptobutyrimidate.

Also, see, Example 1 for monoclonal antibody production.

### Nucleic Acid Molecules (i.e., Polynucleotide) Encoding Antibody Moieties

The present application further provides isolated nucleic acid molecules comprising polynucleotides that encode one or more chains of the antibody moieties (e.g., anti-PD-L1 antibody moieties) described herein. In some embodiments, a nucleic acid molecule comprises a polynucleotide that encodes a heavy chain or a light chain of an antibody moiety (e.g., anti-PD-L1 antibody moiety). In some embodiments, a nucleic acid molecule comprises both a polynucleotide that encodes a heavy chain and a polynucleotide that encodes a light chain, of an antibody moiety (e.g., anti-PD-L1 antibody moiety). In some embodiments, a first nucleic acid molecule comprises a first polynucleotide that encodes a heavy chain and a second nucleic acid molecule comprises a second polynucleotide that encodes a light chain. In some embodiments, a nucleic acid molecule encoding an scFv (e.g., anti-PD-L1 scFv) is provided.

In some such embodiments, the heavy chain and the light chain are expressed from one nucleic acid molecule, or from two separate nucleic acid molecules, as two separate polypeptides. In some embodiments, such as when an antibody is an scFv, a single polynucleotide encodes a single polypeptide comprising both a heavy chain and a light chain linked together.

In some embodiments, a polynucleotide encoding a heavy chain or light chain of an antibody moiety (e.g., anti-PD-L1 antibody moiety) comprises a nucleotide sequence that encodes a leader sequence, which, when translated, is located at the N terminus of the heavy chain or light chain. As discussed above, the leader sequence may be the native heavy or light chain leader sequence, or may be another heterologous leader sequence.

Nucleic acid molecules may be constructed using recombinant DNA techniques conventional in the art. In some embodiments, a nucleic acid molecule is an expression vector that is suitable for expression in a selected host cell.

### Vectors

Vectors comprising polynucleotides that encode the heavy chains and/or light chains of any one of the antibody moieties described herein (e.g., anti-PD-L1 antibody moieties) are provided. Vectors comprising polynucleotides that encode any of the scFvs described herein (e.g., anti-PD-L1 scFv) are also provided. Such vectors include, but are not limited to, DNA vectors, phage vectors, viral vectors, retroviral vectors, etc. In some embodiments, the vector is selected from the group consisting of a vector, plasmid, bacteriophage, cosmide, S factor, retroelement, retrovirus, virus, artificial chromosome (YAC, BAC or MAC), mini chromosome and a chromosome.

In some embodiments, a vector comprises a first polynucleotide sequence encoding a heavy chain and a second polynucleotide sequence encoding a light chain. In some embodiments, the heavy chain and light chain are expressed from the vector as two separate polypeptides. In some embodiments, the heavy chain and light chain are expressed as part of a single polypeptide, such as, for example, when the antibody is an scFv.

In some embodiments, the vector is an expression vector. In some embodiments, expression vectors containing a nucleic acid sequence encoding a polypeptide described herein (or an active derivative or fragment thereof), operably linked to at least one regulatory sequence. Many expression vectors are commercially available, and other suitable vectors can be readily prepared by the skilled artisan. “Operably linked” is intended to mean that the nucleotide sequence is linked to a regulatory sequence in a manner which allows expression of the nucleic acid sequence. Regulatory sequences are art-recognized and are selected to produce the polypeptide or active derivative or fragment thereof. Accordingly, the term “regulatory sequence” includes promoters, enhancers, and other expression control elements which are described in Goeddel, Gene Expression Technology: Methods in Enzymology 185, Academic Press, San Diego, Calif. (1990). For example, the native regulatory sequences or regulatory sequences native to organism can be employed.

In some embodiments, a first vector comprises a polynucleotide that encodes a heavy chain and a second vector comprises a polynucleotide that encodes a light chain. In some embodiments, the first vector and second vector are transfected into host cells in similar amounts (such as similar molar amounts or similar mass amounts). In some embodiments, a mole- or mass-ratio of between 5:1 and 1:5 of the first vector and the second vector is transfected into host cells. In some embodiments, a mass ratio of between 1:1 and 1:5 for the vector encoding the heavy chain and the vector encoding the light chain is used. In some embodiments, a mass ratio of 1:2 for the vector encoding the heavy chain and the vector encoding the light chain is used.

In some embodiments, a vector is selected that is optimized for expression of polypeptides in CHO or CHO-derived cells, or in NSO cells. Exemplary such vectors are described, e.g., in Running Deer et al., Biotechnol. Prog. 20:880-889 (2004).

### Host Cells

In some embodiments, the antibody moieties described herein (e.g., anti-PD-L1 antibody moieties) may be expressed in prokaryotic cells, such as bacterial cells; or in eukaryotic cells, such as fungal cells (such as yeast), plant cells, insect cells, and mammalian cells. Such expression may be carried out, for example, according to procedures known in the art. Exemplary eukaryotic cells that may be used to express polypeptides include, but are not limited to, COS cells, including COS 7 cells; 293 cells, including 293-6E cells; CHO cells, including CHO-S, DG44. Lec13 CHO cells, and FUT8 CHO cells; PER.C6® cells (Crucell); and NSO cells. In some embodiments, the antibody moieties described herein (e.g., anti-PD-L1 antibody moieties) may be expressed in yeast. See, e.g., U.S. Publication No. US 2006/0270045 A1. In some embodiments, a particular eukaryotic host cell is selected based on its ability to make desired post-translational modifications to the heavy chains and/or light chains of the antibody moiety. For example, in some embodiments, CHO cells produce polypeptides that have a higher level of sialylation than the same polypeptide produced in 293 cells.

Introduction of one or more nucleic acids into a desired host cell may be accomplished by any method, including but not limited to, calcium phosphate transfection, DEAE-dextran mediated transfection, cationic lipid-mediated transfection, electroporation, transduction, infection, etc. Non-limiting exemplary methods are described, e.g., in Sambrook et al., Molecular Cloning, A Laboratory Manual, 3^{rd} ed. Cold Spring Harbor Laboratory Press (2001). Nucleic acids may be transiently or stably transfected in the desired host cells, according to any suitable method.

The invention also provides host cells comprising any of the polynucleotides or vectors described herein. In some embodiments, the invention provides a host cell comprising an anti-PD-L1 antibody. Any host cells capable of over-expressing heterologous DNAs can be used for the purpose of isolating the genes encoding the antibody, polypeptide or protein of interest. Non-limiting examples of mammalian host cells include but not limited to COS, HeLa, and CHO cells. See also PCT Publication No. WO 87/04462. Suitable non-mammalian host cells include prokaryotes (such as E. coli or B. subtillis) and yeast (such as S. cerevisae, S. pombe; or K. lactis). In some embodiments, the host cell is a prokaryotic cell. In some embodiments, the host cell is a bacterial cell. In some embodiments, the cell is a eukaryotic cell. In some embodiments, the host cell is a yeast cell. In some embodiments, the host cell is an animal cell. In some embodiments, the host cell is a mammalian cell. In some embodiments, the host cell is a CHO cell.

In some embodiments, the antibody moiety is produced in a cell-free system. Non-limiting exemplary cell-free systems are described, e.g., in Sitaraman et al., Methods Mol. Biol. 498: 229-44 (2009); Spirin, Trends Biotechnol. 22: 538-45 (2004); Endo et al., Biotechnol. Adv. 21: 695-713 (2003).

### Purification of Antibody Moieties

The antibody moieties (e.g., anti-PD-L1 antibody moieties) may be purified by any suitable method. Such methods include, but are not limited to, the use of affinity matrices or hydrophobic interaction chromatography. Suitable affinity ligands include the ROR1 ECD and ligands that bind antibody constant regions. For example, a Protein A, Protein G, Protein A/G, or an antibody affinity column may be used to bind the constant region and to purify an antibody moiety comprising an Fc fragment. Hydrophobic interactive chromatography, for example, a butyl or phenyl column, may also suitable for purifying some polypeptides such as antibodies. Ion exchange chromatography (e.g. anion exchange chromatography and/or cation exchange chromatography) may also suitable for purifying some polypeptides such as antibodies. Mixed-mode chromatography (e.g. reversed phase/anion exchange, reversed phase/cation exchange, hydrophilic interaction/anion exchange, hydrophilic interaction/cation exchange, etc.) may also suitable for purifying some polypeptides such as antibodies. Many methods of purifying polypeptides are known in the art.

### Polynucleotide, Nucleic Acid Construct, Vector, Host Cell, and Culture Medium

### Polynucleotide

In some embodiments, there is provided a polynucleotide encoding any one of the antibody agents or antibody moeities described herein. In some embodiments, there is provided a polynucleotide prepared using any one of the methods as described herein.

In some embodiments, the polynucleotide is a DNA. In some embodiments, the polynucleotide is an RNA. In some embodiments, the RNA is an mRNA. In some embodiments, the polynucleotide comprises a nucleic acid sequence having at least about 80% (such as at least about any one of 80%, 85%, 90%, 95%, 96%, 97%, 98%, or 99%) sequence identity to the nucleic acid sequence of any one of SEQ ID Nos: 2, 4, 6, 8, 10, 12, 14, 16, 18, and 20.

### Nucleic Acid Construct

In some embodiments, there is provided a nucleic acid construct comprising any one of the polynucleotides described herein. In some embodiments, there is provided a nucleic acid construct prepared using any method described herein.

In some embodiments, the nucleic acid construct further comprises a promoter operably linked to the polynucleotide. In some embodiments, the polynucleotide corresponds to a gene, wherein the promoter is a wild-type promoter for the gene.

### Vector

In some embodiments, there is provided a vector comprising any nucleic acid construct described herein. In some embodiments, there is provided a vector prepared using any method described herein.

In some embodiments, the vector is an expression vector.

In some embodiments, the vector is selected from the group consisting of a plasmid, bacteriophage, cosmide, S factor, retroelement, retrovirus, virus, artificial chromosome (e.g., YAC, BAC or MAC), mini chromosome and a chromosome.

### Host Cell

In some embodiments, there is provided a host cell comprising any polypeptide, nucleic acid construct and/or vector described herein. In some embodiments, there is provided a vector prepared using any method described herein.

In some embodiments, the host cell is capable of producing any antibody moiety ad described herein under a fermentation condition.

In some embodiments, the host cell is a prokaryotic cell. In some embodiments, the host cell is a bacterial cell (e.g., E. coli cells). In some embodiments, the host cell is a eukaryotic cell. In some embodiments, the host cell is a yeast cell. In some embodiments, the host cell is an animal cell. In some embodiments, the host cell is a mammalian cell. In some embodiments, the host cell is a CHO cell.

### Culture Medium

In some embodiments, there is provided a culture medium comprising any antibody moiety, polynucleotide, nucleic acid construct, vector, and/or host cell described herein. In some embodiments, there is provided a culture medium prepared using any method described herein.

In some embodiments, the medium comprises hypoxanthine, aminopterin, and/or thymidine (e.g., HAT medium). In some embodiments, the medium does not comprise serum. In some embodiments, the medium comprises serum. In some embodiments, the medium is a D-MEM or RPMI-1640 medium.

### VI. Compositions, Kits and Articles of Manufacture

Also provided herein are compositions (such as formulations) comprising any one of the antibody agents (such as anti-PD-L1 antibody agents) and/or label compound (e.g., radionuclide compound) described herein, nucleic acid encoding the antibody moieties (e.g., anti-PD-L1 antibody moieties), vector comprising the nucleic acid encoding the antibody moieties, or host cells comprising the nucleic acid or vector.

Suitable formulations of the antibody agents (such as anti-PD-L1 antibody agents) and/or label compound (e.g., radionuclide compound) described herein can be obtained by mixing the antibody agents (such as anti-PD-L1 antibody agents) and/or label compound (e.g., radionuclide compound) having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propylparaben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). Lyophilized formulations adapted for subcutaneous administration are described in WO97/04801. Such lyophilized formulations may be reconstituted with a suitable diluent to a high protein concentration and the reconstituted formulation may be administered subcutaneously to the individual to be imaged, diagnosed, or treated herein.

The formulations to be used for in vivo administration must be sterile. This is readily accomplished by, e.g., filtration through sterile filtration membranes.

Also provided are kits comprising any one of the antibody agents (such as anti-PD-L1 antibody agents) and/or label compound (e.g., radionuclide compound) described herein. The kits may be useful for any of the methods of imaging, diagnosis and treatment described herein.

In some embodiments, there is provided a kit comprising an antibody moiety specifically binding an immune checkpoint ligand (e.g., PD-L1 or a PD-L1 like ligand), and a conjugation moiety (e.g., TCO or Tz).

In some embodiments, there is provided a kit comprising an label compound comprising a label (e.g., a radionuclide, e.g., ⁶⁸Ga), and a conjugation moiety (e.g., TCO or Tz). In some embodiments, the conjugation moiety further comprises a chelating agent (e.g., NOTA, DOTA or derivatives thereof) that chelates the radionuclide.

In some embodiments, the kit further comprises a device capable of delivering the antibody agent and/or label compound. One type of device, for applications such as parenteral delivery, is a syringe that is used to inject the composition into the body of a subject. Inhalation devices may also be used for certain applications.

In some embodiments, the kit further comprises a therapeutic agent for treating a disease or condition, e.g., cancer, infectious disease, autoimmune disease, or metabolic disease. In some embodiments, the therapeutic agent is an inhibitor of the immune checkpoint ligand or receptor thereof. In some embodiments, the therapeutic agent is a radiolabeled molecule specifically binding the immune checkpoint ligand or receptor thereof.

The kits of the present application are in suitable packaging. Suitable packaging includes, but is not limited to, vials, bottles, jars, flexible packaging (e.g., sealed Mylar or plastic bags), and the like. Kits may optionally provide additional components such as buffers and interpretative information.

The present application thus also provides articles of manufacture. The article of manufacture can comprise a container and a label or package insert on or associated with the container. Suitable containers include vials (such as sealed vials), bottles, jars, flexible packaging, and the like. Generally, the container holds a composition, and may have a sterile access port (for example the container may be an intravenous solution bag or a vial having a stopper pierceable by a hypodermic injection needle). The label or package insert indicates that the composition is used for imaging, diagnosing, or treating a particular condition in an individual. The label or package insert will further comprise instructions for administering the composition to the individual and for imaging the individual. The label may indicate directions for reconstitution and/or use. The container holding the composition may be a multi-use vial, which allows for repeat administrations (e.g. from 2-6 administrations) of the reconstituted formulation. Package insert refers to instructions customarily included in commercial packages of diagnostic products that contain information about the indications, usage, dosage, administration, contraindications and/or warnings concerning the use of such diagnostic products. Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The kits or article of manufacture may include multiple unit doses of the compositions and instructions for use, packaged in quantities sufficient for storage and use in pharmacies, for example, hospital pharmacies and compounding pharmacies.

Those skilled in the art will recognize that several embodiments are possible within the scope and spirit of this invention. The invention will now be described in greater detail by reference to the following non-limiting examples. The following examples further illustrate the invention but, of course, should not be construed as in any way limiting its scope.

### VII. Use of the Composition

The present application also proves uses of the composition (e.g., the antibody agents and/or label compounds as described herein) for the manufacture of for any of the imaging, diagnosing, treatment, or other methods as described herein.

### VIII. Computer System

The present application also provides computer system for facilitating any of the methods described herein. In some embodiments, there is provided a computer system comprising: an input unit that receives a request from a user to determine the distribution of an immune checkpoint ligand in an individual; one or more computer processors operatively coupled to the input unit, wherein the one or more computer processors are individually or collectively programmed to: a) receiving a set of data comprising a signal of an imaging agent at a tissue of interest in an individual; b) presenting the data in a readable manner or generating an analysis of the data.

The imaging agent may be produced by any method described herein. In some embodiments, the imaging agent is produced in vivo via a conjugation of a first conjugation moiety and a second conjugation moiety after: i) an administration of an effective amount of any antibody agent comprising an antibody moiety and the first conjugation moiety into the individual, wherein the antibody moiety specifically binds the immune checkpoint ligand, and ii) a subsequent administration of an effective amount of a radionuclide compound comprising a radionuclide and the second conjugation moiety into the individual. It is comtemplated that the antibody agent and the label compound can be any antibody agent and label compound described herein.

In some embodiments, the computer system further comprising an output unit for reporting the data or the analysis to the user.

The set of data described herein can represent any number of files of any type, such as text files, image files, audio/video files (such as high-definition files), etc. The files can be non-compressed or compressed. When a file is non-compressed, it can first be compressed before being converted into a binary string. For example, the file can be compressed into a LZMA file (e.g., A.lzma) using the Lempel-Ziv-Markov Chain algorithm. In some embodiments, two or more files (such as three, four, five, six, and more files) are first grouped together, for example, into a TAR file (e.g., A.tar), and the TAR file is further compressed into a LZMA file (e.g., A.tar.lzma).

In some embodiments, the set of data is obtained by imaging the imaging agent in the individual with a non-invasive imaging technique. In some embodiments, the imaging is carried out between about 30 minutes and about 24 hours after administration of the label compound (e.g., radionuclide compound). For example, the imaging is carried out between about 30 minutes and about 1 hour, about 1 hours to about 2 hours, about 2 hours to about 3 hours, about 3 hours to about 4 hours, about 4 hours to about 6 hours, about 6 hours to about 9 hours, about 9 hours to about 12 hours, about 12 hours to about 18 hours, or about 18 hours to about 24 hours after the administration of the label compound (e.g., radionuclide compound). In some embodiments, the imaging is carried out at least about 30 minutes, 1 hours, 2 hours, 3 hours, 4 hours, 6 hours, 9 hours, 12 hours, 18 hours after the administration of the label compound (e.g., radionuclide compound). In some embodiments, the imaging is carried out within 30 minutes, 1 hours, 2 hours, 3 hours, 4 hours, 6 hours, 9 hours, 12 hours, 18 hours after the administration of the label compound (e.g., radionuclide compound). In some embodiments, the non-invasive imaging technique comprises single photon emission computed tomography (SPECT) imaging or positron emission tomography (PET) imaging. In some embodiments, the non-invasive imaging technique comprises or further comprises computed tomography imaging, magnetic resonance imaging, chemical luminescence imaging, or electrochemical luminescence imaging.

In some embodiments, the analysis comprises comparing the set of data with a second set of data comprising a signal of a second imaging agent at a second tissue of interest in a second individual. In some embodiments, the second tissue of interest involved in the second set of data is the same tissue of interest involved in the first set of data. In some embodiments, the second individual is the same individual as the individual involved in the first set of data. In some embodiments, the second set of data is obtained by imaging the second individual with an imaging technique described herein. In some embodiments, the imaging of the second individual is carried out prior to the administration of the antibody agent and/or the label compound. In some embodiments, the imaging of the second individual is carrier out within about 100 hours, for example, about 48 hours, 24 hours, 12 hours, 6 hours, or 2 hours prior to the administration of the antibody agent and/or the label compound.

In some embodiments, the first conjugation moiety and the second conjugation moiety each comprises a member of a click chemistry pair, and are conjugated to each other via click chemistry. In some embodiments, the chemistry pair is selected from the group consisting of a TCO-Tz pair, an azide-alkyne pair, an alkyne-nitrone pair, an alkene-tetrazole pair, and an isonitrile-tetrazine pair. In some embodiments, the first conjugation moiety is a trans-cyclooctene (TCO) and the second conjugation moiety is a tetrazine (Tz), or the first conjugation moiety is a Tz and the second conjugation moiety is a TCO.

In some embodiments, the label compound is administered between about 1 hour and about 100 hours after the administration of the antibody agent.

In some embodiments, the output unit comprises: a textual output; a graphical output; an auditory output; or any combination thereof.

In some embodiments, the individual has a solid tumor, a hematological malignancy, an infectious disease, autoimmune disease, or metabolic disease.

Unless otherwise contradictory, any limitations, features, embodiments, definitions, method steps, conditions, and the like described elsewhere are also applicable to the computer system.

### Exemplary Embodiments

Embodiment 1. An antibody agent comprising an antibody moiety, wherein the antibody moiety is an antibody or an antigen-binding fragment thereof, wherein the antibody or antigen-binding fragment thereof comprises a heavy chain variable region (V_{H}) and a light chain variable region (V_{L}), wherein: a) the V_{H} comprises an HC-CDR1 comprising the amino acid sequence of SEQ ID NO: 41, an HC-CDR2 comprising the amino acid sequence of SEQ ID NO: 42, and an HC-CDR3 comprising the amino acid sequence of SEQ ID NO: 43, or a variant thereof comprising up to a total of about 5 amino acid substitutions in the HC-CDRs; and b) the V_{L} comprises an LC-CDR1 comprising the amino acid sequence of SEQ ID NO: 44, an LC-CDR2 comprising the amino acid sequence of SEQ ID NO: 45, and an LC-CDR3 comprising the amino acid sequence of SEQ ID NO: 46, or a variant thereof comprising up to a total of about 5 amino acid substitutions in the LC-CDRs.

Embodiment 2. The antibody agent of embodiment 1, wherein: a) the VH comprises an HC-CDR1 comprising the amino acid sequence of SEQ ID NO: 41, an HC-CDR2 comprising the amino acid sequence of SEQ ID NO: 42, and an HC-CDR3 comprising the amino acid sequence of SEQ ID NO: 43; and/or b) the V_{L} comprises an LC-CDR1 comprising the amino acid sequence of SEQ ID NO: 44, an LC-CDR2 comprising the amino acid sequence of SEQ ID NO: 45, and an LC-CDR3 comprising the amino acid sequence of SEQ ID NO: 46.

Embodiment 3. The antibody agent of embodiment 1 or 2, wherein: a) the V_{H} comprises an amino acid sequence having at least about 80% sequence identity to the amino acid sequence of any one of SEQ ID NOs: 1, 5, 9, 11, and 13; and b) the V_{L} comprises an amino acid sequence having at least about 80% sequence identity to the amino acid sequence of any one of SEQ ID NOs: 3, 7, 15, 17 and 19.

Embodiment 4. The antibody agent of embodiment 3, wherein the antibody moiety comprises an amino acid sequence having at least about 80% sequence identity to the amino acid sequence of SEQ ID NO: 21 or 23.

Embodiment 5. An antibody agent comprising an antibody moiety, wherein said antibody moiety is an antibody or an antigen-binding fragment thereof, wherein said antibody or antigen-binding fragment thereof specifically binds to PD-L1 competitively with an anti-PD-L1 antibody comprising a VH and a VL, wherein: a) the V_{H} comprises an HC-CDR1 comprising the amino acid sequence of SEQ ID NO: 41, an HC-CDR2 comprising the amino acid sequence of SEQ ID NO: 42, and an HC-CDR3 comprising the amino acid sequence of SEQ ID NO: 43; and b) the V_{L} comprises an LC-CDR1 comprising the amino acid sequence of SEQ ID NO: 44, an LC-CDR2 comprising the amino acid sequence of SEQ ID NO: 45, and an LC-CDR3 comprising the amino acid sequence of SEQ ID NO: 46.

Embodiment 6. The antibody agent of any one of embodiments 1-5, wherein the antibody moiety is selected from the group consisting of a single-chain Fv (scFv), a Fab, a Fab′, a F(ab′)2, an Fv fragment, a disulfide stabilized Fv fragment (dsFv), a (dsFv)₂, a V_{H}H, a Fv-Fc fusion, a scFv-Fc fusion, a scFv-Fv fusion, a diabody, a tribody, and a tetrabody.

Embodiment 7. The antibody agent of any one of embodiments 1-6, wherein the antibody moiety has an isotype selected from the group consisting of an IgG, an IgM, an IgA, an IgD, and an IgE.

Embodiment 8. The antibody agent of any one of embodiments 1-7, wherein the antibody moiety comprises a scFv fused to an Fc fragment.

Embodiment 9. The antibody agent of embodiment 8, wherein the Fc fragment comprises H310A and H435Q mutations, wherein the amino acid positions are based on the Kabat numbering system.

Embodiment 10. The antibody agent of any one of embodiments 1-9, wherein the antibody agent further comprises a first conjugation moiety, wherein the first conjugation moiety is capable of being conjugated to a second conjugation moiety in vivo.

Embodiment 11. The antibody agent of embodiment 10, wherein the first conjugation moiety comprises a member of a click chemistry pair.

Embodiment 12. The antibody agent of embodiment 11, wherein the click chemistry pair is selected from the group consisting of a trans-cyclooctene (TCO)-tetrazine (Tz) pair, an azide-alkyne pair, an alkyne-nitrone pair, an alkene-tetrazole pair, and an isonitrile-tetrazine pair.

Embodiment 13. A polynucleotide encoding the antibody moiety of the antibody agent according to any one of embodiments 1-12.

Embodiment 14. A nucleic acid construct, comprising the polynucleotide according to embodiment 1, optionally further comprising a promoter in operative connection with the polynucleotide.

Embodiment 15. A vector comprising the nucleic acid construct according to embodiment 14.

Embodiment 16. A host cell comprising the polynucleotide according to embodiment 13, the nucleic acid construct according embodiment 14, or the vector according to embodiment 15.

Embodiment 17. A culture medium comprising the antibody moiety of the antibody agent according to embodiments 1 to 9, the polynucleotide according to embodiment 13, the nucleic acid construct according to embodiment 14, the vector according to embodiment 15, or the host cell according to embodiment 16.

Embodiment 18. A method of determining the distribution of an immune checkpoint ligand in an individual, comprising: administering to the individual an effective amount of an antibody agent comprising an antibody moiety and a first conjugation moiety, wherein the antibody moiety specifically binds the immune checkpoint ligand; subsequently administering to the individual an effective amount of a radionuclide compound comprising a radionuclide and a second conjugation moiety, wherein the first conjugation moiety and the second conjugation moiety is conjugated to each other in vivo to provide an imaging agent; and imaging the imaging agent in the individual with a non-invasive imaging technique.

Embodiment 19. The method of embodiment 18, wherein the first conjugation moiety and the second conjugation moiety each comprises a member of a click chemistry pair, and are conjugated to each other via click chemistry.

Embodiment 20. The method of embodiment 19, wherein the chemistry pair is selected from the group consisting of a TCO-Tz pair, an azide-alkyne pair, an alkyne-nitrone pair, an alkene-tetrazole pair, and an isonitrile-tetrazine pair.

Embodiment 21. The method of embodiment 19, wherein the first conjugation moiety is a trans-cyclooctene (TCO) and the second conjugation moiety is a tetrazine (Tz), or the first conjugation moiety is a Tz and the second conjugation moiety is a TCO.

Embodiment 22. The method of any one of embodiments 18-21, wherein the radionuclide compound is administered between about 1 hour and about 100 hours after the administration of the antibody agent.

Embodiment 23. The method of any one of embodiments 18-22, wherein the effective amount of the antibody agent is between about 0.1 mg/kg and about 100 mg/kg.

Embodiment 24. The method of any one of embodiments 18-23, wherein the effective amount of the radionuclide is between about 10 uCi and 500 uCi.

Embodiment 25. The method of any one of embodiments 18-24, wherein the imaging is carried out between about 30 minutes and about 24 hours after administration of the radionuclide compound.

Embodiment 26. The method of any one of embodiments 18-25, further comprising determining the expression level of the immune checkpoint ligand in a tissue of interest in the individual based on signals emitted by the imaging agent from the tissue.

Embodiment 27. The method of any one of embodiments 18-26, wherein the imaging agent is cleared from the individual within between about 10 minutes and about seven days.

Embodiment 28. The method of any one of embodiments 18-27, wherein the half-life of the antibody agent is between about 10 minutes and about 8 days in serum.

Embodiment 29. The method of any one of embodiments 18-28, wherein the binding between the antibody moiety and the immune checkpoint ligand has a K_{D} between about 9×10⁻¹⁰ M and about 1×10⁻⁸ M.

Embodiment 30. The method of any one of embodiments 18-29, wherein the molecular weight of the antibody moiety is no more than about 400 kDa.

Embodiment 31. The method of any one of embodiments 18-30, wherein the antibody moiety cross-reacts with the immune checkpoint ligand from a non-human mammal.

Embodiment 32. The method of any one of embodiments 18-31, wherein the antibody moiety is humanized.

Embodiment 33. The method of any one of embodiments 18-32, wherein the antibody moiety is stable at acidic pH.

Embodiment 34. The method of any one of embodiments 18-33, wherein the antibody moiety has a melting temperature (Tm) of about 55-70° C.

Embodiment 35. The method of any one of embodiments 18-34, wherein the antibody moiety is selected from the group consisting of a single-chain Fv (scFv), a Fab, a Fab′, a F(ab′)2, an Fv fragment, a disulfide stabilized Fv fragment (dsFv), a (dsFv)₂, a V_{H}H, a Fv-Fc fusion, a scFv-Fc fusion, a scFv-Fv fusion, a diabody, a tribody, and a tetrabody.

Embodiment 36. The method of any one of embodiments 18-35, wherein the antibody moiety comprises an scFv fused to an Fc fragment.

Embodiment 37. The method of embodiment 36, wherein the Fc fragment is a human IgG1 Fc fragment.

Embodiment 38. The method of embodiment 36 or 37, wherein the Fc fragment comprises H310A and H435Q mutations, wherein the amino acid positions are based on the Kabat numbering system.

Embodiment 39. The method of any one of embodiments 18-38, wherein the immune checkpoint ligand is PD-L1 or a PD-L1 like ligand.

Embodiment 40. The method of any one of embodiments 18-39, wherein the antibody moiety comprises heavy chain variable region (V_{H}) and a light chain variable region (V_{L}), wherein: a) the V_{H} comprises a heavy chain complementarity determining region 1 (HC-CDR1) comprising the amino acid sequence of SEQ ID NO: 41, an HC-CDR2 comprising the amino acid sequence of SEQ ID NO: 42, and an HC-CDR3 comprising the amino acid sequence of SEQ ID NO: 43, or a variant thereof comprising up to a total of about 5 amino acid substitutions in the HC-CDRs; and b) the V_{L} comprises a light chain complementarity determining region 1 (LC-CDR1) comprising the amino acid sequence of SEQ ID NO: 44, an LC-CDR2 comprising the amino acid sequence of SEQ ID NO: 45, and an LC-CDR3 comprising the amino acid sequence of SEQ ID NO: 46, or a variant thereof comprising up to a total of about 5 amino acid substitutions in the LC-CDRs.

Embodiment 41. The method of any one of embodiments 18-40, wherein the tissue of interest is negative for the immune checkpoint ligand based on an immunohistochemistry (IHC) assay.

Embodiment 42. The method of any one of embodiments 18-41, the tissue of interest has a low expression level of the immune checkpoint ligand.

Embodiment 43. The method of any one of embodiments 18-42, wherein the tissue of interest only expresses the immune checkpoint ligand upon infiltration of immune cells.

Embodiment 44. The method of any one of embodiments 18-43, further comprising imaging the individual over a period of time.

Embodiment 45. The method of any one of embodiments 18-44, wherein the radionuclide is selected from the group consisting of ⁶⁴Cu, ¹⁸F, ⁶⁷Ga, ⁶⁸Ga, ¹¹¹In, ¹⁷⁷Lu, ⁹⁰Y, ⁸⁹Zr, ⁶¹Cu, ⁶²Cu, ⁶⁷Cu, ¹⁹F, ⁶⁶Ga, ⁷²Ga, ⁴⁴Sc, ⁴⁷Sc, ⁸⁶Y, ⁸⁸Y and ⁴⁵Ti.

Embodiment 46. The method of embodiment 45, wherein the radionuclide is ⁶⁸Ga.

Embodiment 47. The method of any one of embodiments 18-46, wherein the radionuclide compound comprises a chelating compound that chelates the radionuclide.

Embodiment 48. The method of embodiment 47, wherein the chelating compound is 1,4,7-triazacyclononane-1,4,7-trisacetic acid (NOTA), 1,4,7,10-tetraazacyclododecane-1,4,7,10-tetraacetic acid (DOTA) or a derivative thereof.

Embodiment 49. The method of any one of embodiments 18-48, wherein the non-invasive imaging technique comprises single photon emission computed tomography (SPECT) imaging or positron emission tomography (PET) imaging.

Embodiment 50. The method of any one of embodiments 18-49, wherein the non-invasive imaging technique comprises computed tomography imaging, magnetic resonance imaging, chemical luminescence imaging, or electrochemical luminescence imaging.

Embodiment 51. The method of any one of embodiments 18-50, wherein the antibody agent is administered intravenously, intraperitoneally, intramuscularly, subcutaneously, or orally.

Embodiment 52. The method of any one of embodiments 18-51, wherein the radionuclide compound is administered intravenously, intraperitoneally, intramuscularly, subcutaneously, or orally.

Embodiment 53. The method of any one of embodiments 18-52, wherein the individual has a solid tumor, a hematological malignancy, an infectious disease, autoimmune disease, or metabolic disease.

Embodiment 54. A method of diagnosing an individual having a disease or condition, comprising: determining the distribution of an immune checkpoint ligand in the individual using the method of any one of embodiments 18-53; and diagnosing the individual as positive for the immune checkpoint ligand if signal of the imaging agent is detected at a tissue of interest, or diagnosing the individual as negative for the immune checkpoint ligand if signal of the imaging agent is not detected at a tissue of interest.

Embodiment 55. A method of treating an individual having a disease or condition, comprising: diagnosing the individual using the method of embodiment 54; and administering to the individual an effective amount of a therapeutic agent targeting the immune checkpoint ligand, if the individual is diagnosed as positive for the immune checkpoint ligand.

Embodiment 56. A kit comprising: an antibody agent comprising an antibody moiety and a first conjugation moiety, wherein the antibody moiety specifically binds an immune checkpoint ligand; and a radionuclide compound comprising a radionuclide and a second conjugation moiety, wherein the first conjugation moiety and the second conjugation moiety are capable of conjugating with each other in vivo.

Embodiment 57. A computer system comprising: an input unit that receives a request from a user to determine the distribution of an immune checkpoint ligand in an individual; one or more computer processors operatively coupled to the input unit, wherein the one or more computer processors are individually or collectively programmed to: receiving a set of data comprising a signal of an imaging agent at a tissue of interest in an individual, wherein the imaging agent is produced in vivo via a conjugation of a first conjugation moiety and a second conjugation moiety after: an administration of an effective amount of an antibody agent comprising an antibody moiety and the first conjugation moiety into the individual, wherein the antibody moiety specifically binds the immune checkpoint ligand, and a subsequent administration an effective amount of a radionuclide compound comprising a radionuclide and the second conjugation moiety into the individual; presenting the data in a readable manner or generating an analysis of the data.

### EXAMPLES

The examples below are intended to be purely exemplary of the invention and should therefore not be considered to limit the invention in any way. The following examples and detailed description are offered by way of illustration and not by way of limitation.

### Example 1: Preparation and Characterization of Monoclonal Antibodies Against Human PD-L1

### Immunization

8-10 weeks old female PD-L1 deficient mice (H Dong et al. Immunity. 2004 March; 20(3):327-36) were immunized subcutaneously (s.c.) at multiple sites with 200 μl of emulsion comprising 100 μg of hPD-L1mIg fusion protein and complete Freund's adjuvant (CFA) (Sigma-Aldrich). Each animal received two or three boosts with emulsion comprising the same concentration of hPD-L1mIg fusion protein formulated in incomplete Freund's adjuvant (IFA) (Sigma-Aldrich). Blood samples were collected from the animals two weeks after each immunization for serum titer testing. Upon achieving a sufficient titer, the animals received a booster injection with 60 μg of the PD-L1mIg fusion protein in PBS through intraperitoneal injection (i.p.). The animals were sacrificed and their spleens were harvested aseptically 5 days after the booster injection.

Whole spleen was dissociated into single-cell suspensions. Red blood cells were lysed using the ACK buffer. The spleen cells were then mixed with SP2/0-Ag14 myeloma cells (from ATCC) at a 1:1 ratio in 50 ml conical centrifuge tubes. After centrifugation, the supernatant was discarded and cell fusion was induced with 50% polyethylene glycol (Roche). The fused cells were cultured for 8-10 days in the HAT selection medium. The contents in the supernatant were analyzed for their ability to bind to hPD-1-expressing cells using ELISA, and the positive clones were further confirmed using flow cytometry analysis. Subcloning of the positive hybridoma was performed using the limiting dilution technique for 5 times to achieve a pure monoclonal culture.

### Characterization of the Anti-hPD-L1 Monoclonal Antibody

### Binding Specificity of the Anti-hPD-L1 Monoclonal Antibody

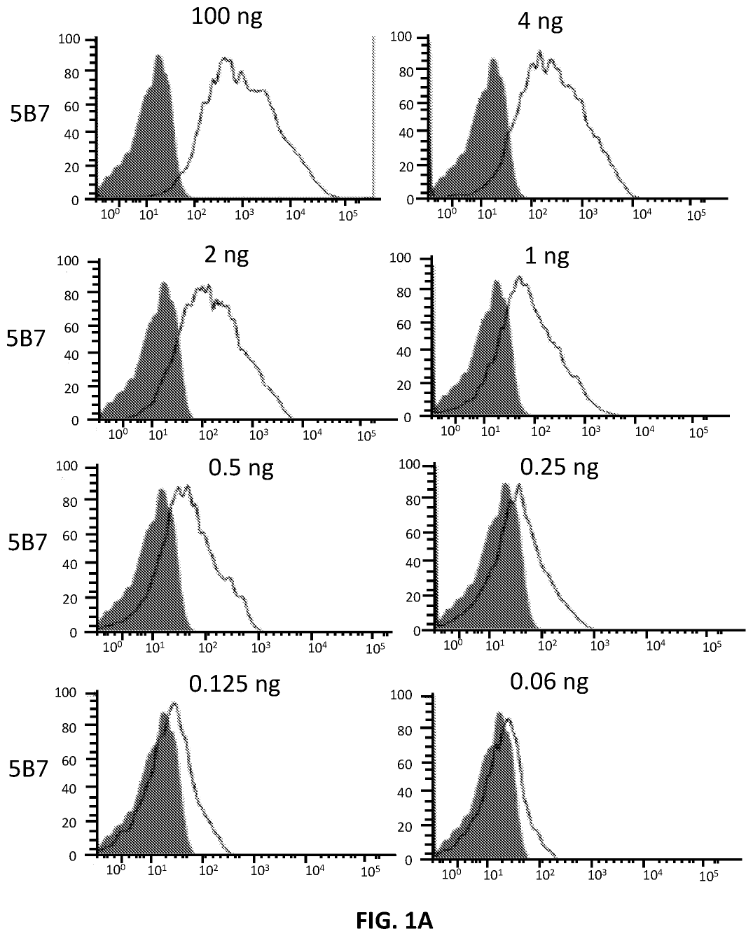

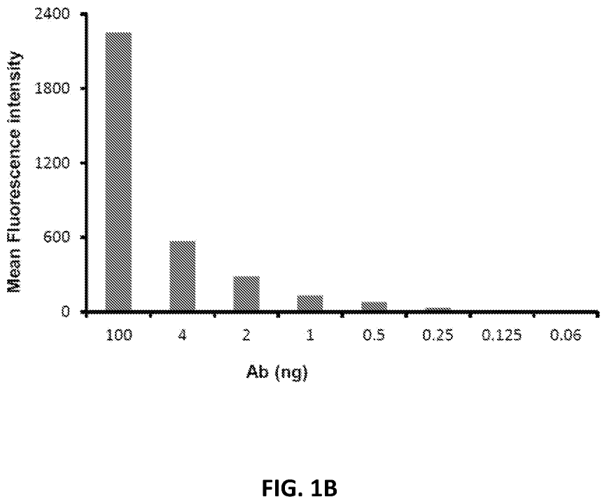

The binding specificity of the anti-hPD-L1 mAb was determined using hPD-L1 transfected CHO cells (CHO/hPD-L1 cells) by flow cytometry (FACSVerse, BD Biosciences). Specifically, CHO/hPD-L1 cells were incubated with increasing amounts of the anti-hPD-L1 mAb 5B7 (0.06 ng, 0.125 ng, 0.25 ng, 0.5 ng, 1 ng, 2 ng, 4 ng, and 100 ng) on ice for 30 minutes. The cells were then washed and further incubated with anti-mIgG-APC (eBioscience) prior to flow cytometry analysis. As shown in FIGS. 1A and 1B, the anti-hPD-L1 mAb 5B7 bound to hPD-L1 with high specificity in a dose-dependent manner.

The isotype of the monoclonal antibody was determined to be IgG1 κ using the Mouse Immunoglobulin Isotyping Kit (BD Biosciences).

### Species Cross-Reactivity

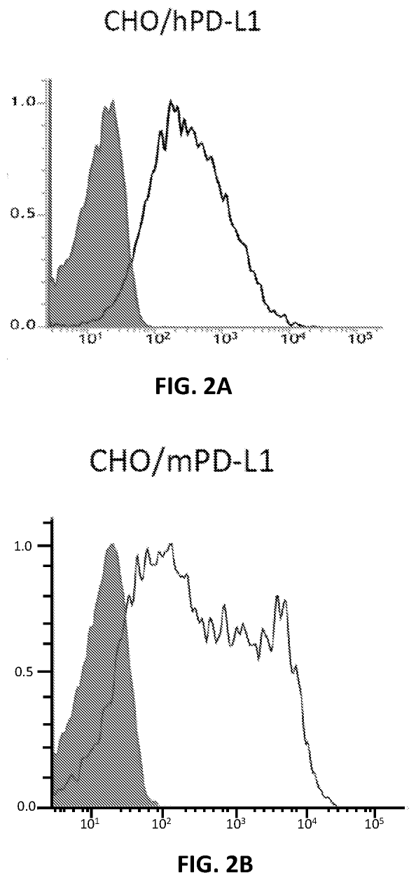

CHO cells transfected with mouse PD-L1 (CHO/mPD-L1) were used to assess the species cross-reactivity of the anti-hPD-L1 mAb with mouse PD-L1. The cells were incubated with the anti-hPD-L1 mAb prior to flow cytometry analysis. FIG. 2A shows the binding specificity of the anti-hPD-L1 mAb to human PD-L1. As shown in FIG. 2B, the anti-hPD-L1 mAb also binds to mouse PD-L1.

### Sequencing of Anti-hPD-L1 Antibody-Producing Hybridoma Cells

To sequence antibody-producing hybridoma cells, 1×10⁷ hybridoma cells were harvested and washed with PBS. Messenger RNAs were extracted from hybridomas using RNeasy Mini Kit (Qiagen). RACE-Readyfirst-Strand cDNAs were synthesized using SMARTer RACE cDNA Amplification Kit (Clontech). Following reverse transcription, 5′ RACE PCR reactions were performed with ready cDNA as template and with 5′ universal primer (UPM) provided by the kit and 3′gene specific primers (GSP1) designed using the mouse IgG1 heavy chain variable region and light chain variable region sequences. RACE products were determined by gel electrophoresis analysis. PCR products were then cloned into a T vector using Zero Blunt TOPO PCR Cloning Kit (Invitrogen). After transformation, the plasmids were verified by sequencing analysis. Sequences of the heavy chain variable region and light chain variable region were analyzed using VBASE2 (worldwide wweb.vbase2.org), and listed in Table 3.

### Example 2: Humanization of Anti-hPD-L1 Antibodies

### Humanization of Anti-hPD-L1 Antibodies

Humanization was performed based on the heavy chain variable region (VH) and light chain variable region (VL) sequences from anti-hPD-L1 hybridoma cells. As an initial step, a mouse-human chimeric mAb comprising the parental mouse VH and VL sequences, the human IgG constant region and the human K chain was generated. Upon characterization of the chimeric antibody, three VH and three VL humanized sequences were designed and used to generate nine humanized antibodies. The VH and VL sequences of the chimeric and humanized anti-hPD-L1 antibodies are listed in Table 3.

### Characterization of Humanized Anti-hPD-L1 Antibodies

### Binding Activities of Humanized Anti-hPD-L1 Antibodies

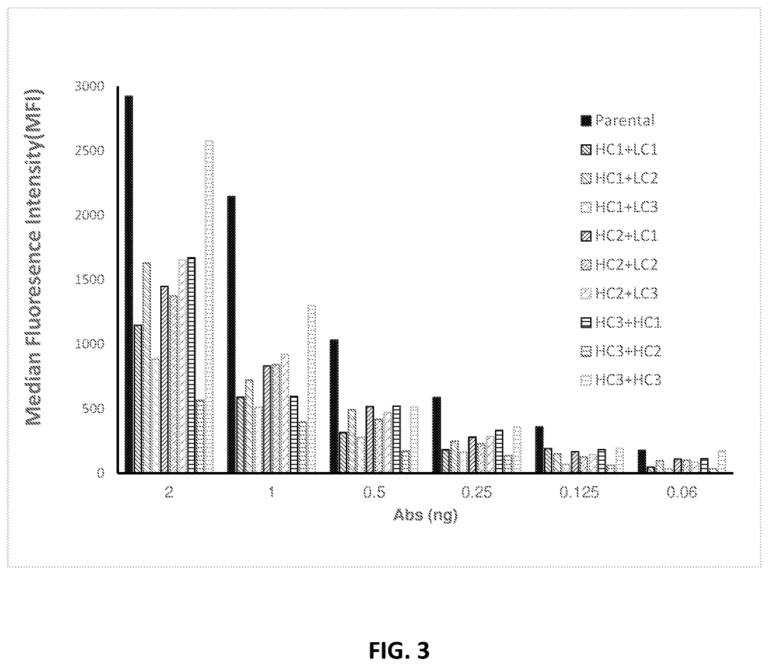

To examine the binding activities of the humanized antibodies as compared to the parental chimeric antibody, CHO/hPD-L1 cells were incubated with the serially diluted parental chimeric antibody or humanized antibodies. The binding affinities were analyzed using flow cytometry, and the results showed that some humanized antibodies demonstrated higher binding affinities to hPD-L1 as compared to the parental chimeric antibody, while other humanized antibodies demonstrated similar or slightly lower binding affinities as compared to the parental chimeric antibody (FIG. 3).

### Binding Affinities and Kinetics of the Humanized Anti-hPD-L1 Antibodies

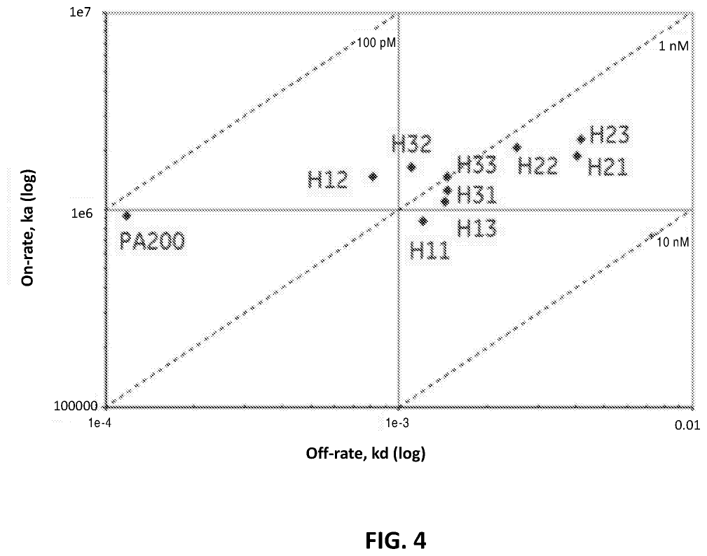

The binding affinities and kinetics between the humanized anti-hPD-L1 mAbs and the hPD-L1 protein were assessed with Biacore T200 (GE Healthcare Life Sciences). The hPD-L1mIg protein was immobilized on the sensor chip CM5 by amine coupling. The parental and humanized antibodies were injected at a concentration gradient of 1, 2, 4, 8, 16, and 32 nM. Binding kinetic parameters, including association rate (Kₒₙ), dissociation rate (K_{off}), and affinity constant (K_{D}) were determined by full kinetic analysis. As shown in Table 6, there were no significant differences in the association rate (Kₒₙ) among the parental antibody and the humanized antibodies. As shown in Table 6 and FIG. 4, the antibodies with dissociation rates (K_{off}) from the lowest to the highest were: parental (PA200), H1+L2 (H12), H3+L2 (H32), H1+L1 (H11), H1+L3 (H13), H3+L1 (H31), H3+L3 (H33), H2+L2 (H22), H2+L1 (H21), and H2+L3 (H23). As shown in Table 6, the antibodies with affinity constant (KD) from the highest to the lowest were: parental, H1+L2, H3+L2, H3+L3, H3+L1, H2+L2, H1+L3, H1+L1, H2+L3, and H2+L1.

TABLE 6
Kₒₙ, K_{off} and K_{D} values of the parental and humanized antibodies

| Antibody | Kₒₙ (1/Ms) | K_{off} (1/s) | K_{D} (M) |
|---|---|---|---|
| anti-PD-L1 parental | 9.243E+5 | 1.180E−4 | 1.276E−10 |
| anti-PD-L1 HC1 + LC1 | 8.686E+5 | 0.001200 | 1.381E−9 |
| anti-PD-L1 HC1 + LC2 | 1.456E+6 | 8.114E−4 | 5.572E−10 |
| anti-PD-L1 HC1 + LC3 | 1.100E+6 | 0.001430 | 1.301E−9 |
| anti-PD-L1 HC2 + LC1 | 1.863E+6 | 0.004058 | 2.178E−9 |
| anti-PD-L1 HC2 + LC2 | 2.049E+6 | 0.002515 | 1.227E−9 |
| anti-PD-L1 HC2 + LC3 | 2.265E+6 | 0.004171 | 1.841E−9 |
| anti-PD-L1 HC3 + LC1 | 1.250E+6 | 0.001464 | 1.171E−9 |
| anti-PD-L1 HC3 + LC2 | 1.646E+6 | 0.001096 | 6.659E−10 |
| anti-PD-L1 HC3 + LC3 | 1.461E+6 | 0.001465 | 1.003E−9 |

### Example 3: Preparation and Characterization of Anti-hPD-L1 scFv-hFc Antibodies

Sequence Design and Synthesis of scFv-hFc Antibodies

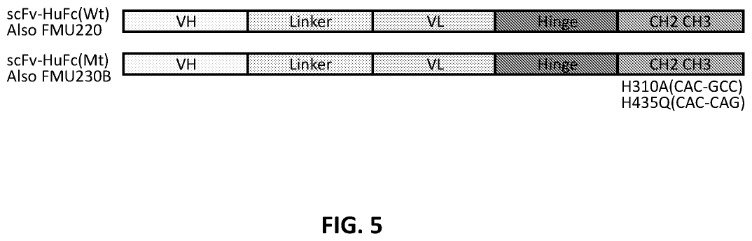

The heavy chain variable region (VH) and light chain variable region (VL) of the humanized anti-hPD-L1 antibody variant 2 was used to generate the scFv-hFc antibody. Specifically, the heavy chain and light chain were connected by a linker, which was followed by a hinge sequence (GACAAGACCCACACCTGCCCTCCCTGCCCC, SEQ ID NO: 50) and a human immunoglobulin IgG1 Fc portion (CH2-CH3 region). Additionally, H310A (i.e., CAC to GCC) and H435Q (i.e., CAC to CAG) mutations were introduced into the CH2 and CH3 regions for rapid clearance of the antibody in vivo (FIG. 5). The sequences of the scFv-hFc antibodies with the wild type CH2-CH3 regions (scFv-hFc Wt) and with the mutant CH2-CH3 regions (scFv-hFc Mt) are shown in Table 5.

The DNA sequences of scFv-hFc Wt and scFv-hFc Mt were cloned into pcDNA3.3 vectors respectively and used to transiently transfected ExPi 293 cells. The proteins from the cell culture supernatant were purified with protein G sepharose column (GE healthcare) for functional analysis.

Characterization of scFv-hFc Antibodies

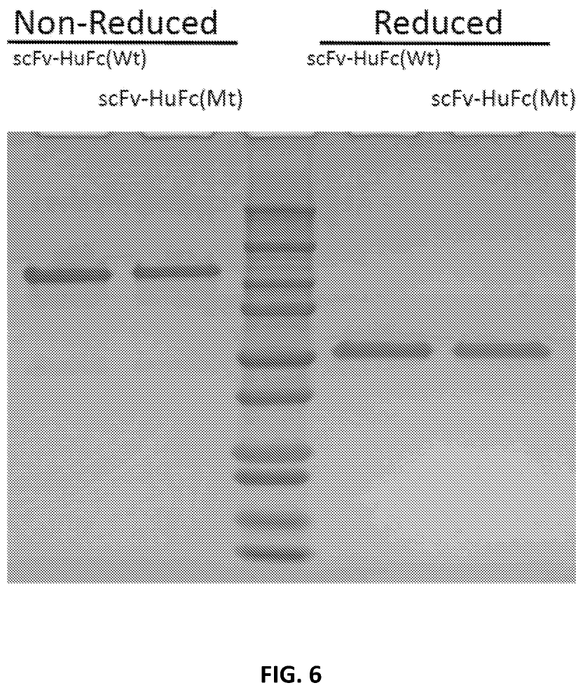

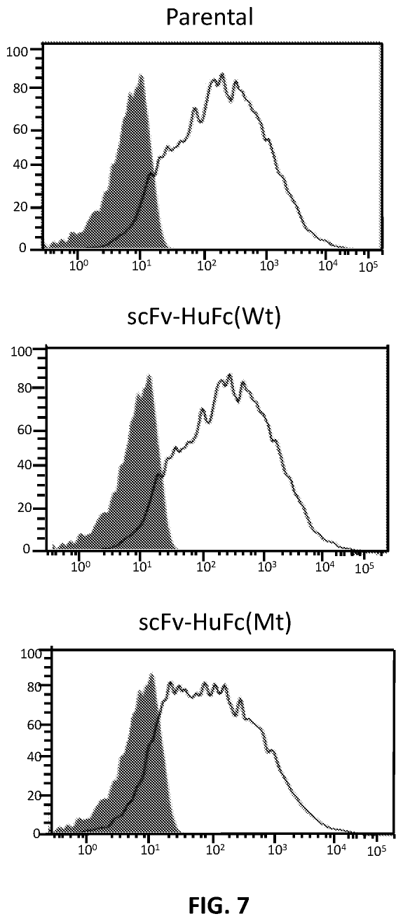

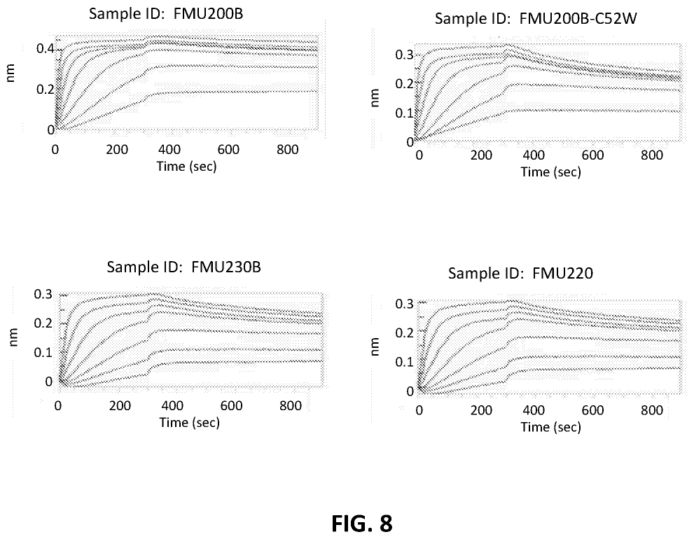

The anti-hPD-L1 scFv-hFc Wt and scFv-hFc Mt antibodies were identified by SDS Page Gel Electroporation. As shown in FIG. 6, the scFv-hFc antibodies were identified in both the reduced and non-reduced conditions on the SDS-PAGE gel. The binding affinities of the scFv-hFc antibodies to hPD-L1, as compared to that of the parental antibody were examined by FACS and the results are shown in FIG. 7. As shown in the histograms, both scFv-hFc Wt and scFv-hFc Mt demonstrated similar binding affinities as the parental antibody. The binding affinities and kinetics of the scFv-hFc antibodies (scFv-hFc Wt and scFv-hFc Mt) to hPD-L1, as compared to that of the parental antibody (anti-PD-L1 IgG1), were further analyzed using the Fortebio Octet system. See FIG. 8. Table 7 shows the binding affinities and kinetics parameters of anti-PD-L1 IgG1, anti-PD-L1 IgG1-C52W (i.e., an anti-PD-L1 antibody having an IgG1 Fc region with a C52W mutation), scFv-hFc Mt and scFv-hFc Wt.

TABLE 7
Binding kinetics parameters of anti-hPD-L1
scFv-hFc and parental control antibodies

| Sample | K_{D} (M) | Kₒₙ (1/Ms) | K_{off} (1/s) | R² |
|---|---|---|---|---|
| anti-PD-L1 IgG1 | 1.54E−11 | 1.77E+5 | 2.72E−5 | 0.9834 |
| anti-PD-L1 IgG1 C52W | 3.40E−10 | 1.33E+5 | 4.53E−4 | 0.9732 |
| anti-PD-L1 scFv-hFc Wt | 3.48E−10 | 6.98E+6 | 2.43E−4 | 0.97 |
| anti-PD-L1 scFv-hFc Mt | 2.32E−10 | 1.66E+6 | 3.85E−4 | 0.9812 |

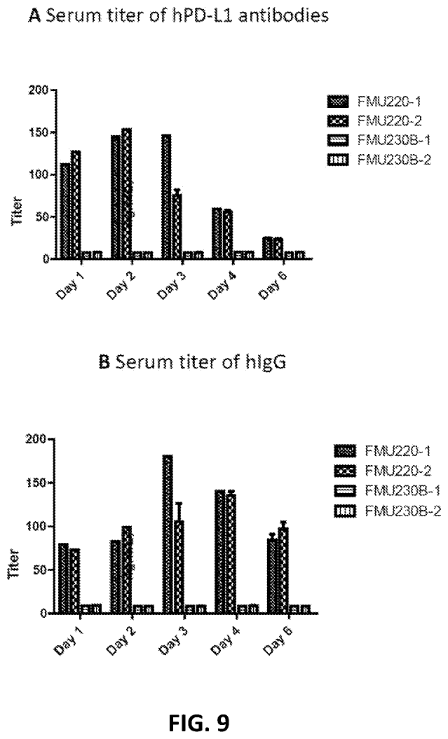

Pharmacokinetics studies were performed by injection of scFv-hFc Wt and scFv-hFc Mt antibodies in vivo, followed by measuring of the serum titers of the scFv-hFc antibodies and hIgG on Day 1, 2, 3, 4 and 6 after injection. As shown in FIGS. 9A and 9B, scFv-hFc Wt showed higher serum titer of the antibody and of hIgG as compared to scFv-hFc Mt.

### Example 4: Labeling of ⁶⁸Ga-DOTA-Tz

0.1N HCl was used to wash off ⁶⁸Ga, and 3.8 mCi/1 mL ⁶⁸Ga was obtained. The pH was adjusted to 4 by adding 93 μL 1.25M sodium acetate. Various volumes (0.5, 2, 5 and 15 L) of DOTA-(PEG)₁₀-Tz (2 mg/ml) was tested and added to 350 μL ⁶⁸Ga. Reagents were incubated for 10 min at different temperatures (25, 37, 60, 80, 100° C.). The labeling efficiency was measured with thin layer chromatography (TLC) using 0.1M sodium citrate as spreading solution.

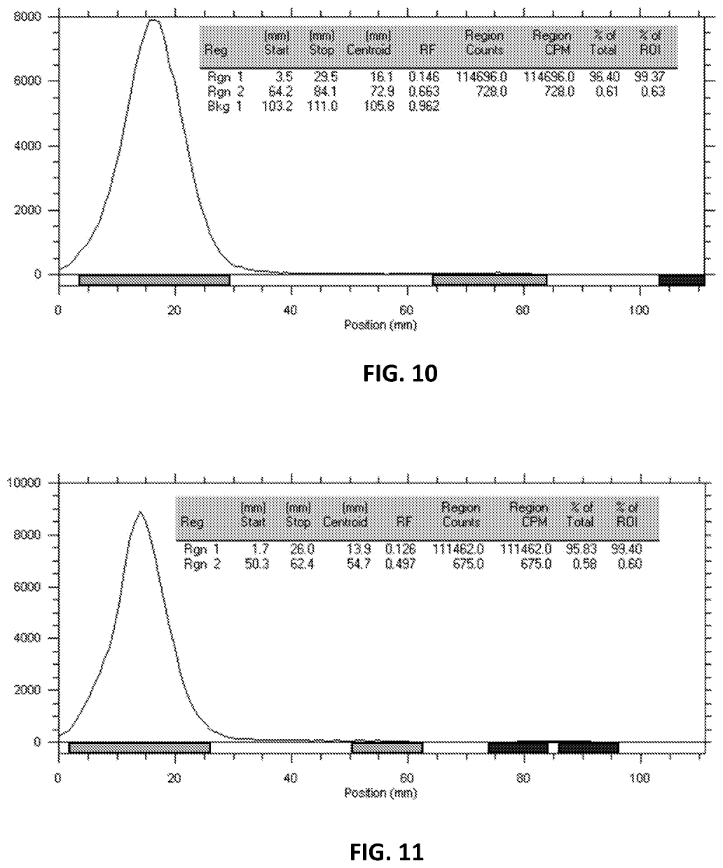

The effects of temperatures on labeling rates were investigated. The labeling rates at 25, 37, 60, 80, or 100° C. were 80%, 80%, 89%, 99% and 99%, respectively. FIG. 10 shows a representative labeling rate at 100° C.

The effects of various volumes of DOTA-(PEG)₁₀-Tz on labeling rates were also investigated. The labeling rates at 100° C. using 0.5, 2 or 5 μL of DOTA-(PEG)₁₀-Tz were 97%, 99%, 100%, respectively. A representative labeling rate of 2 μL DOTA-(PEG)₁₀-Tz at 100° C. is shown in FIG. 11.

It is concluded that 100° C. incubation temperature and 2 μL DOTA-(PEG)₁₀-Tz is a suitable labeling condition.

### Example 5: Conjugation of TCO to PD-L1 ScFv-Fc

FMU-230B or FMU-220 with a volume of 200 μL were mixed with 250 μL PBS buffer. The mixture was then loaded into a Centricon tube and centrifuged at 16000 rpm for 24 min and concentrated the solution within a volume of 100 μL. PBS with a volume of 100 μL was added and the entire content was transfer into 1.5 mL centrifuge tube. 1 μL NHS-(PEG)₄-TCO (diluted with 6 μL DMSO) was added and mixed, followed by the addition of 10 μL 1M Na₂CO₃, and incubated at 37° C. for 60 minutes. The content was transferred into a Centricon tube with the addition of 200 μL PBS and centrifuged at 16000 rpm for 24 min. Additional 350 μL PBS was added and centrifuged at 16000 rpm for 24 min. PBS with a volume of 100 μL was added and the content was transferred to 1.5 mL centrifuge tube and stored at 4° C.

The binding of TCO labeled FMU0220 or FMU-230B to cells expressing PD-L1 (e.g., MC-38-B7H1) was detected when FMU-230B or FMU-220 were initially mixed with PBS buffer, but not some other buffer such as 0.05M NaHCO₃—Na₂CO₃ buffer solution.

### Binding of ⁶⁸Ga-DOTA-Tz-TCO-PD-L1 to MC-38-B7H1 Cells

MC-38-B7H1 and MC-38 cells were cultured in plates at the density of 5×10⁵/ml. 2 μL TCO-PD-L1 (100 μL TCO-FMU-220 or TCO-FMU-230B in 400 μL PBS) were added the second day and incubated at 37° C. for 1 hour in serum free medium. Then 1 μL ⁶⁸Ga-Tz was added and incubated for 30 min. Cells were then washed with PBS and treated with 200 μL trypsin digestion for 5 min. Cells were suspended in 200 μL PBS and radioactivity was quantified with γ-counter.

⁶⁸Ga-Tz radioactivity was detected in MC-38-B7H1 cells treated with either ⁶⁸Ga-DOTA-Tz-TCO-FMU-230B and ⁶⁸Ga-DOTA-Tz-TCO-FMU-220, but with different characteristics.

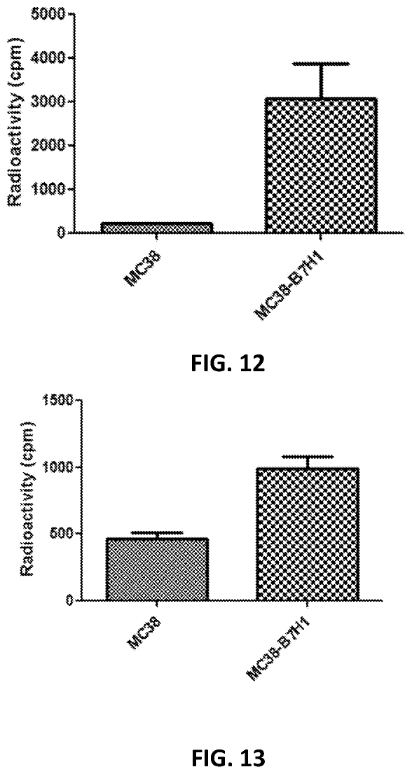

Under ⁶⁸Ga-DOTA-Tz-TCO-FMU-230B treatments, little radioactivity was detected for MC-38 cells (FIG. 12), suggesting the binding was mediated specifically by PD-L1.

Under ⁶⁸Ga-DOTA-Tz-TCO-FMU-220 treatments, significantly more radioactivity on MC-38-B7H1 cells was also detected as compared to MC-38 cells, suggesting the specific binding to PD-L1 (FIG. 13). When compared to the results of ⁶⁸Ga-DOTA-Tz-TCO-FMU-230B, a relatively lower radioactivity on MC38-B7H1 cells and a relatively higher radioactivity on MC-38 cells were observed. This decreased binding specificity may be due to repeated freeze-thaw of FMU-220.

### Example 6: In Vivo Live Imaging of ⁶⁸Ga-DOTA-Tz-TCO-PD-L1-ScFv-Fc

MC38-PD-L1 cells were cultured and counted after trypsin digestion. 1×10⁶ MC38-PD-L1 cells were injected into the right axilla of five 6-8 week old female mice, and 1×10⁶ MC38-PD-L1 cells were injected into the left axilla of the mice. The tumors reached a diameter of about 0.5 cm after 6 days, at which point the animals received TCO-FMU-220 or TCO-FMU-230B as described below.

### I. Imaging of ⁶⁸Ga-DOTA-Tz-TCO-FMU-220

### A. After 3 Hours of Pre-Targeting

Mice were injected via tail veil with TCO-FMU-220 50 uL (about 100 ug) in 150 uL PBS. Three hours later, ⁶⁸Ga-Tz was injected into the tail vein. Mice were then imaged at 60 minutes and 150 minutes after injection. The following tissue organs were visualized: the bladder, the kidney, the tumor, and the heart. After 3 hours of pre-targeting, the uptake in tumors in the right axilla was high. Some animals had cardiac imaging and relatively high uptake in bladder and/or kidney. Representative images at 60 minutes and 150 minutes were shown in FIG. 14.

### B. After 24 Hours of Pre-Targeting

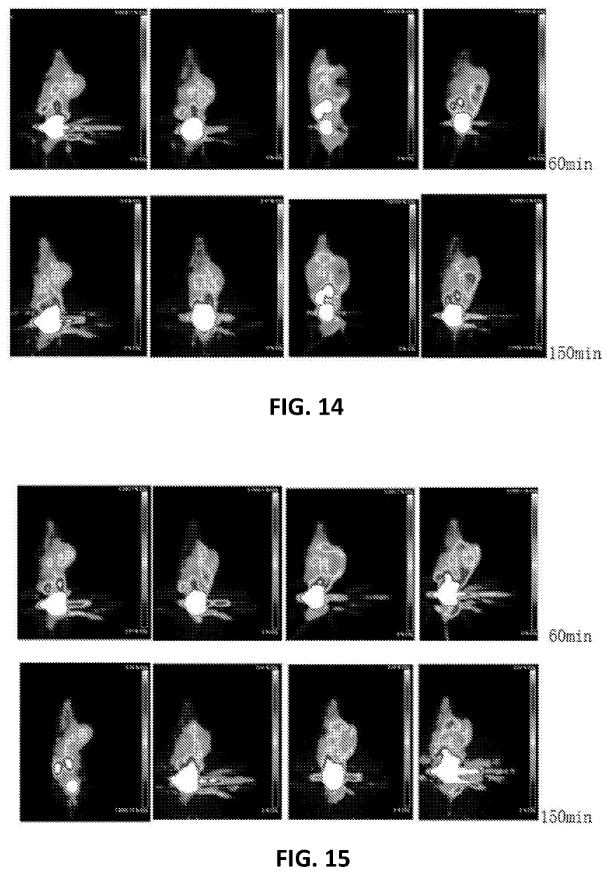

Mice were injected via tail veil with TCO-FMU-220 50 uL in 150 uL PBS. Twenty-four hours later, ⁶⁸Ga-Tz was injected into the tail vein. The mice were then imaged at 60 minutes and 150 minutes after injection. The following tissue organs were visualized: the bladder, the kidney, the tumor, and the heart. After 24 hours of pre-targeting, the uptake in tumors in the right axilla was high. Some animals had cardiac imaging and relatively high uptake in bladder and/or kidney. Representative images at 60 minutes and 150 minutes were shown in FIG. 15.

### C. After 48 Hours of Pre-Targeting

The mice were injected via tail veil with TCO-FMU-220 50 uL in 150 uL PBS. Then 48 hours later, ⁶⁸Ga-Tz was injected into the tail vein. Mice were then imaged at 60 minutes and 150 minutes after injection. The following tissue organs were visualized: the bladder, the kidney, the tumor, and the heart. After 48 hours of pre-targeting, the uptake in tumors in the right axilla was high. Some animals had cardiac imaging and relatively high uptake in bladder and/or kidney. Representative images show that uptake at 60 minutes is higher than uptake at 150 minutes. See FIG. 16.

In conclusion, in vivo imaging of TCO-FMU-220 antibody can be visualized at least during 3-48 hours after pre-targeting. One suitable condition is to pre-target the animal for three to twenty-four hours, followed by imaging the animal 60 minutes after ⁶⁸Ga-Tz injection.

### II. Imaging of ⁶⁸Ga-DOTA-Tz-TCO-FMU-230B

### A. After 3 Hours of Pre-Targeting

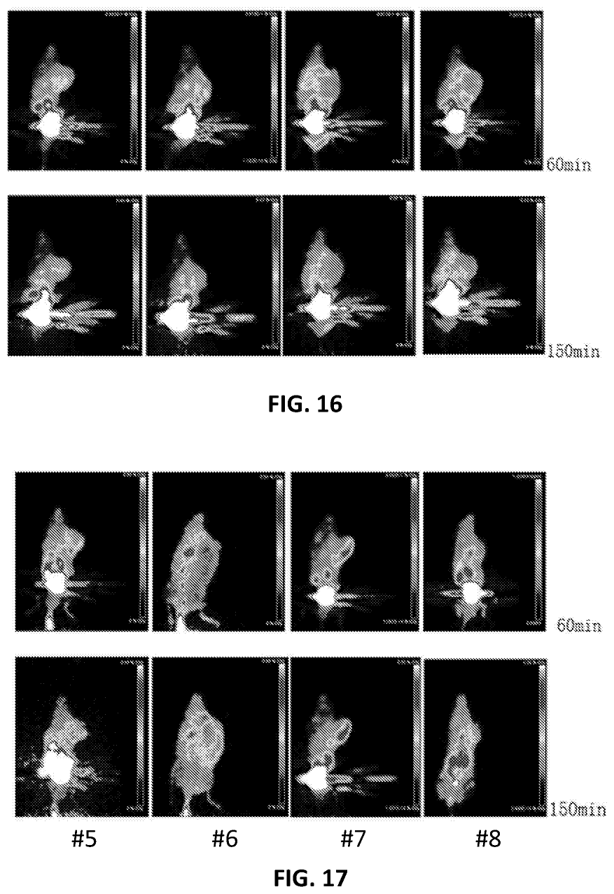

Mice were injected via tail veil with TCO-FMU-230B 50 uL (about 120 ug) in 150 uL PBS. Three hours later, ⁶⁸Ga-Tz was injected into the tail vein. Mice were then imaged at 60 minutes and 150 minutes after injection. The following tissue organs were visualized: the bladder, the kidney, the tumor, and the heart. Representative images are shown in FIG. 17. After pre-targeted for 3 hours, the tumor uptake in the right axilla was higher (especially in animal #7). Some animals had the uptake in the heart and liver (animal #8). In some animals, the uptake of bladder was higher, followed by the kidneys. Animals #5 and #6 had drug leakage. Animal #6 had uptake at lymph node at 60 minutes. Tumor can be clearly visualized at 150 minutes time point.

### B. After 24 Hours of Pre-Targeting

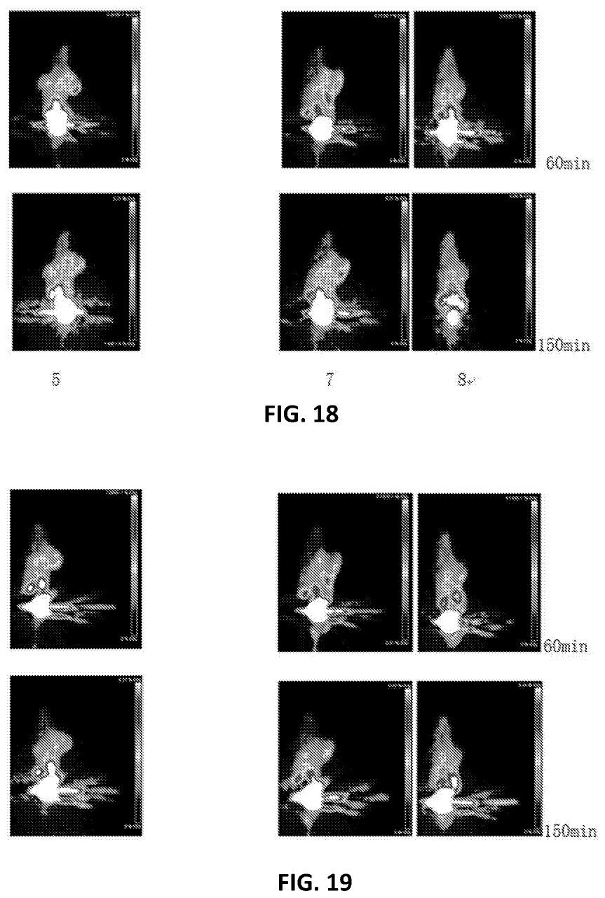

Mice were injected via tail veil with TCO-FMU-230B 50 uL in 150 uL PBS. Twenty-four hours later, ⁶⁸Ga-Tz was injected into the tail vein. Mice were then imaged at 60 minutes and 150 minutes after injection. The following tissue organs were visualized: the bladder, the kidney, the tumor, and the heart. Representative images are shown in FIG. 18. After 24 hours of pretargeting, the tumor uptake was high. Some animals had cardiac imaging and high bladder uptake, followed by kidney. The imaging at 60 minutes is more obvious than that at 150 minutes. There is no image for mouse #6 due to its death.

### C. After 48 Hours of Pre-Targeting

Mice were injected via tail veil with TCO-FMU-230B 50 uL in 150 uL PBS. Forty-eight hours later, ⁶⁸Ga-Tz was injected into the tail vein. Mice were then imaged at 60 minutes and 150 minutes after injection. The following tissue organs were visualized: the bladder, the kidney, the tumor, and the heart. Representative images are shown in FIG. 19. After 48 hours of pre-targeting, the uptake in tumors was high. Some animals had cardiac imaging and relatively high uptake in bladder and kidney. The imaging at 60 minutes is more obvious than that at 150 minutes. There is no image for mouse #6 due to its death.

In conclusion, after pretargeting TCO-FMU-230B antibody for 3-48 hours, imagines were visualized. One suitable condition for imaging is to pretarget the mouse for 3-24 hours, followed by imaging the mouse 60 minutes after ⁶⁸Ga-Tz injection.

In summary, a high affinity, thermo-stable antibody and modified antibody-like fusion protein (scFv-Fc) targeting human PD-L1 as in vivo imaging agent was engineered. It was successfully conjugated with TCO. With the labeling of ⁶⁸Ga-DOTA-Tz, the binding of TCO-PD-L1 with PD-L1 expressing cells (e.g., MC-38-B7H1 cells) can be visualized with in vivo live imaging, and the distribution of PD-L1 in an individual can be successfully visualized.

What is claimed is:

1. An antibody agent comprising an antibody moiety, wherein the antibody moiety is an antibody or an antigen-binding fragment thereof, wherein the antibody or antigen-binding fragment thereof comprises a heavy chain variable region (V_{H}) and a light chain variable region (V_{L}), wherein:
a) the V_{H} comprises an HC-CDR1 comprising the amino acid sequence of SEQ ID NO: 41, an HC-CDR2 comprising the amino acid sequence of SEQ ID NO: 42, and an HC-CDR3 comprising the amino acid sequence of SEQ ID NO: 43, or a variant thereof comprising up to a total of about 5 amino acid substitutions in the HC-CDRs; and
b) the V_{L} comprises an LC-CDR1 comprising the amino acid sequence of SEQ ID NO: 44, an LC-CDR2 comprising the amino acid sequence of SEQ ID NO: 45, and an LC-CDR3 comprising the amino acid sequence of SEQ ID NO: 46, or a variant thereof comprising up to a total of about 5 amino acid substitutions in the LC-CDRs.

2. The antibody agent of claim 1, wherein:
a) the VH comprises an HC-CDR1 comprising the amino acid sequence of SEQ ID NO: 41, an HC-CDR2 comprising the amino acid sequence of SEQ ID NO: 42, and an HC-CDR3 comprising the amino acid sequence of SEQ ID NO: 43; and/or
b) the V_{L} comprises an LC-CDR1 comprising the amino acid sequence of SEQ ID NO: 44, an LC-CDR2 comprising the amino acid sequence of SEQ ID NO: 45, and an LC-CDR3 comprising the amino acid sequence of SEQ ID NO: 46.

3. The antibody agent of claim 1 or 2, wherein:
a) the V_{H} comprises an amino acid sequence having at least about 80% sequence identity to the amino acid sequence of any one of SEQ ID NOs: 1, 5, 9, 11, and 13; and
b) the V_{L} comprises an amino acid sequence having at least about 80% sequence identity to the amino acid sequence of any one of SEQ ID NOs: 3, 7, 15, 17 and 19.

4. The antibody agent of claim 3, wherein the antibody moiety comprises an amino acid sequence having at least about 80% sequence identity to the amino acid sequence of SEQ ID NO: 21 or 23.

5. An antibody agent comprising an antibody moiety, wherein said antibody moiety is an antibody or an antigen-binding fragment thereof, wherein said antibody or antigen-binding fragment thereof specifically binds to PD-L1 competitively with an anti-PD-L1 antibody comprising a VH and a VL, wherein:
a) the V_{H} comprises an HC-CDR1 comprising the amino acid sequence of SEQ ID NO: 41, an HC-CDR2 comprising the amino acid sequence of SEQ ID NO: 42, and an HC-CDR3 comprising the amino acid sequence of SEQ ID NO: 43; and
b) the V_{L} comprises an LC-CDR1 comprising the amino acid sequence of SEQ ID NO: 44, an LC-CDR2 comprising the amino acid sequence of SEQ ID NO: 45, and an LC-CDR3 comprising the amino acid sequence of SEQ ID NO: 46.

6. The antibody agent of any one of claims 1-5, wherein the antibody moiety is selected from the group consisting of a single-chain Fv (scFv), a Fab, a Fab′, a F(ab′)2, an Fv fragment, a disulfide stabilized Fv fragment (dsFv), a (dsFv)₂, a V_{H}H, a Fv-Fc fusion, a scFv-Fc fusion, a scFv-Fv fusion, a diabody, a tribody, and a tetrabody.

7. The antibody agent of any one of claims 1-6, wherein the antibody moiety has an isotype selected from the group consisting of an IgG, an IgM, an IgA, an IgD, and an IgE.

8. The antibody agent of any one of claims 1-7, wherein the antibody moiety comprises a scFv fused to an Fc fragment.

9. The antibody agent of claim 8, wherein the Fc fragment comprises H310A and H435Q mutations, wherein the amino acid positions are based on the Kabat numbering system.

10. The antibody agent of any one of claims 1-9, wherein the antibody agent further comprises a first conjugation moiety, wherein the first conjugation moiety is capable of being conjugated to a second conjugation moiety in vivo.

11. The antibody agent of claim 10, wherein the first conjugation moiety comprises a member of a click chemistry pair.

12. The antibody agent of claim 11, wherein the click chemistry pair is selected from the group consisting of a trans-cyclooctene (TCO)-tetrazine (Tz) pair, an azide-alkyne pair, an alkyne-nitrone pair, an alkene-tetrazole pair, and an isonitrile-tetrazine pair.

13. A polynucleotide encoding the antibody moiety of the antibody agent according to any one of claims 1-12.

14. A nucleic acid construct, comprising the polynucleotide according to claim 1, optionally further comprising a promoter in operative connection with the polynucleotide.

15. A vector comprising the nucleic acid construct according to claim 14.

16. A host cell comprising the polynucleotide according to claim 13, the nucleic acid construct according claim 14, or the vector according to claim 15.

17. A culture medium comprising the antibody moiety of the antibody agent according to claims 1 to 9, the polynucleotide according to claim 13, the nucleic acid construct according to claim 14, the vector according to claim 15, or the host cell according to claim 16.

18. A method of determining the distribution of an immune checkpoint ligand in an individual, comprising:
(a) administering to the individual an effective amount of an antibody agent comprising an antibody moiety and a first conjugation moiety, wherein the antibody moiety specifically binds the immune checkpoint ligand;
(b) subsequently administering to the individual an effective amount of a radionuclide compound comprising a radionuclide and a second conjugation moiety, wherein the first conjugation moiety and the second conjugation moiety is conjugated to each other in vivo to provide an imaging agent; and
(c) imaging the imaging agent in the individual with a non-invasive imaging technique.

19. The method of claim 18, wherein the first conjugation moiety and the second conjugation moiety each comprises a member of a click chemistry pair, and are conjugated to each other via click chemistry.

20. The method of claim 19, wherein the chemistry pair is selected from the group consisting of a TCO-Tz pair, an azide-alkyne pair, an alkyne-nitrone pair, an alkene-tetrazole pair, and an isonitrile-tetrazine pair.

21. The method of claim 19, wherein the first conjugation moiety is a trans-cyclooctene (TCO) and the second conjugation moiety is a tetrazine (Tz), or the first conjugation moiety is a Tz and the second conjugation moiety is a TCO.

22. The method of any one of claims 18-21, wherein the radionuclide compound is administered between about 1 hour and about 100 hours after the administration of the antibody agent.

23. The method of any one of claims 18-22, wherein the effective amount of the antibody agent is between about 0.1 mg/kg and about 100 mg/kg.

24. The method of any one of claims 18-23, wherein the effective amount of the radionuclide is between about 10 uCi and 500 uCi.

25. The method of any one of claims 18-24, wherein the imaging is carried out between about 30 minutes and about 24 hours after administration of the radionuclide compound.

26. The method of any one of claims 18-25, further comprising determining the expression level of the immune checkpoint ligand in a tissue of interest in the individual based on signals emitted by the imaging agent from the tissue.

27. The method of any one of claims 18-26, wherein the imaging agent is cleared from the individual within between about 10 minutes and about seven days.

28. The method of any one of claims 18-27, wherein the half-life of the antibody agent is between about 10 minutes and about 8 days in serum.

29. The method of any one of claims 18-28, wherein the binding between the antibody moiety and the immune checkpoint ligand has a K_{D} between about 9×10⁻¹⁰ M and about 1×10⁻⁸ M.

30. The method of any one of claims 18-29, wherein the molecular weight of the antibody moiety is no more than about 400 kDa.

31. The method of any one of claims 18-30, wherein the antibody moiety cross-reacts with the immune checkpoint ligand from a non-human mammal.

32. The method of any one of claims 18-31, wherein the antibody moiety is humanized.

33. The method of any one of claims 18-32, wherein the antibody moiety is stable at acidic pH.

34. The method of any one of claims 18-33, wherein the antibody moiety has a melting temperature (Tm) of about 55-70° C.

35. The method of any one of claims 18-34, wherein the antibody moiety is selected from the group consisting of a single-chain Fv (scFv), a Fab, a Fab′, a F(ab′)2, an Fv fragment, a disulfide stabilized Fv fragment (dsFv), a (dsFv)₂, a V_{H}H, a Fv-Fc fusion, a scFv-Fc fusion, a scFv-Fv fusion, a diabody, a tribody, and a tetrabody.

36. The method of any one of claims 18-35, wherein the antibody moiety comprises an scFv fused to an Fc fragment.

37. The method of claim 36, wherein the Fc fragment is a human IgG1 Fc fragment.

38. The method of claim 36 or 37, wherein the Fc fragment comprises H310A and H435Q mutations, wherein the amino acid positions are based on the Kabat numbering system.

39. The method of any one of claims 18-38, wherein the immune checkpoint ligand is PD-L1 or a PD-L1 like ligand.

40. The method of any one of claims 18-39, wherein the antibody moiety comprises heavy chain variable region (V_{H}) and a light chain variable region (V_{L}), wherein:
a) the V_{H} comprises a heavy chain complementarity determining region 1 (HC-CDR1) comprising the amino acid sequence of SEQ ID NO: 41, an HC-CDR2 comprising the amino acid sequence of SEQ ID NO: 42, and an HC-CDR3 comprising the amino acid sequence of SEQ ID NO: 43, or a variant thereof comprising up to a total of about 5 amino acid substitutions in the HC-CDRs; and
b) the V_{L} comprises a light chain complementarity determining region 1 (LC-CDR1) comprising the amino acid sequence of SEQ ID NO: 44, an LC-CDR2 comprising the amino acid sequence of SEQ ID NO: 45, and an LC-CDR3 comprising the amino acid sequence of SEQ ID NO: 46, or a variant thereof comprising up to a total of about 5 amino acid substitutions in the LC-CDRs.

41. The method of any one of claims 18-40, wherein the tissue of interest is negative for the immune checkpoint ligand based on an immunohistochemistry (IHC) assay.

42. The method of any one of claims 18-41, the tissue of interest has a low expression level of the immune checkpoint ligand.

43. The method of any one of claims 18-42, wherein the tissue of interest only expresses the immune checkpoint ligand upon infiltration of immune cells.

44. The method of any one of claims 18-43, further comprising imaging the individual over a period of time.

45. The method of any one of claims 18-44, wherein the radionuclide is selected from the group consisting of ⁶⁴Cu, ¹⁸F, ⁶⁷Ga, ⁶⁸Ga, ¹¹¹In, ¹⁷⁷Lu, ⁹⁰Y, ⁸⁹Zr, ⁶¹Cu, ⁶²Cu, ⁶⁷Cu, ¹⁹F, ⁶⁶Ga, ⁷²Ga, ⁴⁴Sc, ⁴⁷Sc, ⁸⁶Y, ⁸⁸Y and ⁴⁵Ti.

46. The method of claim 45, wherein the radionuclide is ⁶⁸Ga.

47. The method of any one of claims 18-46, wherein the radionuclide compound comprises a chelating compound that chelates the radionuclide.

48. The method of claim 47, wherein the chelating compound is 1,4,7-triazacyclononane-1,4,7-trisacetic acid (NOTA), 1,4,7,10-tetraazacyclododecane-1,4,7,10-tetraacetic acid (DOTA) or a derivative thereof.

49. The method of any one of claims 18-48, wherein the non-invasive imaging technique comprises single photon emission computed tomography (SPECT) imaging or positron emission tomography (PET) imaging.

50. The method of any one of claims 18-49, wherein the non-invasive imaging technique comprises computed tomography imaging, magnetic resonance imaging, chemical luminescence imaging, or electrochemical luminescence imaging.

51. The method of any one of claims 18-50, wherein the antibody agent is administered intravenously, intraperitoneally, intramuscularly, subcutaneously, or orally.

52. The method of any one of claims 18-51, wherein the radionuclide compound is administered intravenously, intraperitoneally, intramuscularly, subcutaneously, or orally.

53. The method of any one of claims 18-52, wherein the individual has a solid tumor, a hematological malignancy, an infectious disease, autoimmune disease, or metabolic disease.

54. A method of diagnosing an individual having a disease or condition, comprising:
(a) determining the distribution of an immune checkpoint ligand in the individual using the method of any one of claims 18-53; and
(b) diagnosing the individual as positive for the immune checkpoint ligand if signal of the imaging agent is detected at a tissue of interest, or diagnosing the individual as negative for the immune checkpoint ligand if signal of the imaging agent is not detected at a tissue of interest.

55. A method of treating an individual having a disease or condition, comprising:
(a) diagnosing the individual using the method of claim 54; and
(b) administering to the individual an effective amount of a therapeutic agent targeting the immune checkpoint ligand, if the individual is diagnosed as positive for the immune checkpoint ligand.

56. A kit comprising:
(a) an antibody agent comprising an antibody moiety and a first conjugation moiety, wherein the antibody moiety specifically binds an immune checkpoint ligand; and
(b) a radionuclide compound comprising a radionuclide and a second conjugation moiety, wherein the first conjugation moiety and the second conjugation moiety are capable of conjugating with each other in vivo.

57. A computer system comprising:
an input unit that receives a request from a user to determine the distribution of an immune checkpoint ligand in an individual;
one or more computer processors operatively coupled to the input unit, wherein the one or more computer processors are individually or collectively programmed to:
a) receiving a set of data comprising a signal of an imaging agent at a tissue of interest in an individual, wherein the imaging agent is produced in vivo via a conjugation of a first conjugation moiety and a second conjugation moiety after:
i. an administration of an effective amount of an antibody agent comprising an antibody moiety and the first conjugation moiety into the individual, wherein the antibody moiety specifically binds the immune checkpoint ligand, and
ii. a subsequent administration an effective amount of a radionuclide compound comprising a radionuclide and the second conjugation moiety into the individual;
b) presenting the data in a readable manner or generating an analysis of the data.
